(19) United States
(12) Patent Application Publication
(10) Pub. No.: US 2022/0160853 A1
(43) Pub. Date: May 26, 2022

(54) CANCER VACCINE COMPOSITIONS AND METHODS FOR USE THEREOF

(71) Applicant: GeoVax, Inc., Smyrna, GA (US)

(72) Inventors: Farshad Guirakhoo, Atlanta, GA (US); Arban Domi, Atlanta, GA (US)

(73) Assignee: GeoVax, Inc., Smyrna, GA (US)

(21) Appl. No.: 17/542,100

(22) Filed: Dec. 3, 2021

Related U.S. Application Data
(63) Continuation of application No. PCT/US2020/035995, filed on Jun. 3, 2020.
(60) Provisional application No. 62/893,500, filed on Aug. 29, 2019.
(60) Provisional application No. 62/856,552, filed on Jun. 3, 2019.

(51) Int. Cl. A61K 39/00; A61P 35/00; C12N 7/04; C12N 15/86

(52) U.S. Cl. CPC A61K 39/00117; A61P 35/00; C12N 7/045; A61K 2039/505; A61K 2039/5158; A61K 2039/53; A61K 2039/55522; C12N 15/86

(57) ABSTRACT

The compositions and methods are described for generating an immune response to a tumor associated antigen (TAA) such as MUC-1, survivin, cyclin B1, HBV, or HPV. The compositions and methods described herein relate to a modified vaccinia Ankara (MVA) vector encoding one or more viral antigens for generating a protective immune response to the tumor associated antigen in the subject to which the vector is administered and optionally, boosting the immune response by administering a tumor associated antigen. The compositions and methods of the present invention are useful both prophylactically and therapeutically and may be used to prevent and/or treat neoplasms and associated diseases.

### CROSS REFERENCE TO RELATED APPLICATIONS

This application is a continuation of International Application No. PCT/US2020/35995, filed with the Patent Cooperation Treaty, U.S. Receiving Office on Jun. 3, 2020, which claims priority to U.S. Provisional Application No. 62/856,552, filed Jun. 3, 2019 and U.S. Provisional Application No. 62/893,500, filed Aug. 29, 2019. The entirety of each of these applications is hereby incorporated by reference herein for all purposes.

### FIELD OF THE INVENTION

The compositions and methods described herein relate to cancer vaccine compositions; methods of manufacture; and methods of use thereof. The compositions and methods disclosed herein are useful both prophylactically and therapeutically.

### INCORPORATION BY REFERENCE

The contents of the text file named “19101-022WO1US1_SequenceListing” which was created on Dec. 3, 2021 and is 28.5 KB in size, are hereby incorporated by reference in their entirety.

### BACKGROUND OF THE INVENTION

In 2016, there will be an estimated 1,735,350 new cancer cases diagnosed and 609,640 cancer deaths in the US (Cancer Facts & FIGS. 2018, American Cancer Society 2018). The standard treatments approaches to treatment, including surgery, radiation, and chemotherapy, are still limited. In particular, many of these treatments are effective only at the earliest stages and/or result in significant damage to normal tissue.

Cancer vaccines have emerged as an approach to treatment of cancer, generating a specific immune response against tumor cells in vivo, producing anti-tumor effects. Cancer vaccines have also been studied to prevent development of cancer in a subject (e.g., prophylactic cancer vaccine). Individually, these vaccines have shown interesting but still limited results.

In particular, cancer vaccines have been used as an approach to preventing cancer caused by oncogenic viruses. Gardasil 9 (human papillomavirus 9-valent vaccine, recombinant; 9vHPV) was approved by FDA for use in 2014. It protects against 9 types of cancer-causing HPV types 6, 11, 16, 18, 31, 33, 45, 52, and 58. The aim of the prophylactic vaccines is to induce humoral immune responses by stimulating the production of neutralizing antibodies specific for the HPV viral capsid proteins, L1 and L2. Yet, HPV still causes about 44,000 cancers per year. Unfortunately, therapeutic vaccines for HPV HPV have shown little efficacy.

Moreover, recent advances in immunotherapies for cancer have demonstrated the potential of checkpoint inhibitors (CPI) to release the ability of exhausted CD8+ T cells to clear and control the re-emergence of metastatic disease. The success of CPI, however, has been limited to only a fraction of target tumors, a limitation that has been hypothesized to reflect the patient not having an exhausted CD8+ T-cell response to the tumor.

There remains a need to improve the efficacy of cancer vaccines, including the immunogenic potential thereof.

### SUMMARY OF THE INVENTION

The compositions and methods described herein are useful for generating an immune response to a tumor associated antigen (TAA) in a subject in need thereof and in certain embodiments, treating cancer in a subject in need thereof including HPV-associated human head and neck tumors.

In a first aspect, disclosed herein is a composition comprising:

a) a recombinant modified vaccinia Ankara (MVA) vector comprising a TAA-encoding sequence and a matrix protein-encoding sequence (matrix protein sequence), and

b) a TAA,

wherein the TAA is survivin or a fragment thereof.

In one embodiment, the recombinant MVA vector further comprises one or more additional transgenes. In a particular embodiment, the transgene encodes an immune checkpoint inhibitor, a cytokine or both.

In a second aspect, disclosed herein is a composition comprising:

a) a recombinant MVA vector comprising a TAA-encoding sequence and a matrix protein-encoding sequence, and

b) a TAA;

wherein the TAA is cyclin B1.

In one embodiment, the recombinant MVA vector further comprises one or more additional transgenes. In a particular embodiment, the transgene encodes an immune checkpoint inhibitor, a cytokine or both.

In a third aspect, disclosed herein is a composition comprising:

a) a recombinant MVA vector comprising a TAA-encoding sequence and a matrix protein-encoding sequence and

b) a TAA;

wherein the TAA is HBV.

In one embodiment, the recombinant MVA vector further comprises one or more additional transgenes. In a particular embodiment, the transgene encodes an immune checkpoint inhibitor, a cytokine or both.

In a fourth aspect, disclosed herein is a composition comprising a recombinant vector comprising a TAA-encoding sequence and a matrix protein-encoding sequence, wherein the TAA is selected from the group consisting of MUC-1, survivin, cyclin B1, and HPV. According to this fourth embodiment, the composition does not comprise TAA apart from that TAA encoded by the vector.

In one embodiment, the recombinant MVA vector further comprises one or more additional transgenes. In a particular embodiment, the transgene encodes an immune checkpoint inhibitor, a cytokine or both.

In a fifth aspect, an immunogenic composition is disclosed comprising a) a recombinant MVA vector or recombinant vaccinia viral (VV) vector comprising a sequence encoding an HPV antigen or fragment thereof and a matrix protein sequence.

In a particular embodiment, the HPV antigens are selected from the group consisting of E2, E6, E7 or combinations thereof.

In one embodiment, the matrix protein is selected from Marburg virus VP40 matrix protein, Ebola virus VP40 matrix protein, human immunodeficiency virus type 1 (HIV-1) matrix protein or Lassa virus matrix Z protein.

In one embodiment, the recombinant MVA vector further comprises one or more additional transgenes. In a particular embodiment, the transgene encodes an immune checkpoint inhibitor, a cytokine or both.

In one embodiment, the vector comprises a TAA-encoding sequence and a matrix protein-encoding sequence inserted into the MVA vector in a natural deletion site, a modified natural deletion site, or between essential or non-essential MVA genes.

In another embodiment, the vector comprises a TAA-encoding sequence and a matrix protein-encoding sequence inserted into the same natural deletion site, a modified natural deletion site, or between the same essential or non-essential MVA genes.

In another embodiment, the vector comprises a TAA-encoding inserted into a deletion site selected from I, II, III, IV, V or VI and a matrix protein-encoding sequence is inserted into a deletion site selected from I, II, III, IV, V or VI.

In another embodiment, the vector comprises a TAA-encoding sequence and a matrix protein-encoding sequence inserted into different natural deletion sites, different modified deletion sites, or between different essential or non-essential MVA genes.

In another embodiment, the vector comprises a TAA-encoding sequence inserted in a first deletion site and a matrix protein-encoding sequence inserted into a second deletion site.

In a particular embodiment, the vector comprises a TAA-encoding sequence inserted between two essential and highly conserved MVA genes; and a matrix protein-encoding sequence inserted into a restructured and modified deletion III.

In a particular embodiment, the vector comprises a matrix protein-encoding sequence inserted between MVA genes, I8R and G1L.

In a particular embodiment, the vector comprises a TAA-encoding sequence inserted between two essential and highly conserved MVA genes to limit the formation of viable deletion mutants.

In a particular embodiment, the vector comprises a TAA-encoding sequence inserted between MVA genes, I8R and G1L.

In one embodiment, the promoter is selected from the group consisting of Pm2H5, Psyn II, and mH5 promoters or combinations thereof.

In one embodiment, the recombinant MVA viral vector expresses the TAA and matrix proteins that assemble into virus like particles (“VLPs”).

In a sixth aspect, a pharmaceutical composition is provided comprising the recombinant MVA vector disclosed herein and/or a TAA and a pharmaceutically acceptable carrier.

In one embodiment, the recombinant MVA vector is formulated for intraperitoneal, intramuscular, intradermal, epidermal, mucosal or intravenous administration.

In a seventh aspect, a method is provided of inducing an immune response to a neoplasm in a subject in need thereof, said method comprising administering to the subject:

a) a composition comprising an immunogenic vector expressing a TAA in an amount sufficient to induce an immune response, or boost a previously induced immune response and

b) a composition comprising a TAA in an amount sufficient to induce an immune response or boost a previously induced immune response, wherein the TAA is selected from the group consisting of survivin, cyclin B1, or HPV.

In one embodiment, the immunogenic vector further comprises one or more additional transgenes. In a particular embodiment, the transgene encodes an immune checkpoint inhibitor, a cytokine or both.

In an eighth aspect, a composition is disclosed comprising an immunogenic vector expressing a TAA (e.g., MUC-1, survivin, cyclin B1, HPV) to the subject in an amount sufficient to induce an immune response, or boost a previously induced immune response. According to this embodiment, there is no need to administer TAA to the subject apart from the TAA antigen expressed by the vector.

In one embodiment, the immunogenic vector further comprises one or more additional transgenes. In a particular embodiment, the transgene encodes an immune checkpoint inhibitor, a cytokine or both.

In one embodiment, the immune response is a humoral immune response, a cellular immune response or a combination thereof.

In a particular embodiment, the immune response comprises production of binding antibodies to the TAA.

In a particular embodiment, the immune response comprises production of neutralizing antibodies to the TAA.

In a particular embodiment, the immune response comprises production of non-neutralizing antibodies to the TAA.

In a particular embodiment, the immune response comprises production of a cell-mediated immune response to the TAA.

In a particular embodiment, the immune response comprises production of neutralizing and non-neutralizing antibodies to the TAA.

In a particular embodiment, the immune response comprises production of neutralizing antibodies and cell-mediated immunity to the TAA.

In a particular embodiment, the immune response comprises production of non-neutralizing antibodies and cell-mediated immunity to the TAA.

In a particular embodiment, the immune response comprises production of neutralizing antibodies, non-neutralizing antibodies, and cell-mediated immunity to the TAA.

In one embodiment, the neoplasm is selected from leukemia (e.g. myeloblastic, promyelocytic, myelomonocytic, monocytic, erythroleukemia, chronic myelocytic (granulocytic) leukemia, and chronic lymphocytic leukemia), lymphoma (e.g., Hodgkin's disease and non-Hodgkin's disease), fibrosarcoma, myxosarcoma, liposarcoma, chondrosarcoma, osteogenic sarcoma, angiosarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilms' tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia.

In another embodiment, the neoplasm is selected from adenocarcinomas (breast, colorectal, pancreatic, other), carcinoid tumor, dhordoma, choriocarcinoma, desmoplastic small round cell tumor (DSRCT), epithelioid sarcoma, follicular dendritic cell sarcoma, interdigitating dendritic cell/reticulum cell sarcoma, lung: type II pneumocyte lesions (type II cell hyperplasia, dysplastic type II cells, apical alveolar hyperplasia), anaplastic large-cell lymphoma, diffuse large B cell lymphoma (variable), plasmablastic lymphoma, primary effusion lymphoma, epithelioid mesotheliomas, myeloma, plasmacytomas, perineurioma, renal cell carcinoma, synovial sarcoma (epithelial areas), fhymic carcinoma (often), meningioma or Paget's disease.

In a ninth aspect, a method of treating cancer is provided comprising administering to a subject in need thereof:

a) an effective amount of a recombinant MVA vector expressing a TAA to prime an immune response, and

b) a TAA in an effective amount to boost an immune response,

wherein the TAA is selected from the group consisting of MUC-1, survivin, cyclin B1, and HPV.

Optionally, the method may further comprise administering c) an oncolytic, armed vaccinia virus to the subject.

In one embodiment, the recombinant MVA vector further comprises one or more additional transgenes. In a particular embodiment, the transgene encodes an immune checkpoint inhibitor, a cytokine or both.

In a particular embodiment, the TAA is MUC-1, and the cancer is a MUC-1+ cancer selected from the group consisting of pancreatic, colorectal, prostate, lunch, and breast cancer.

In another particular embodiment, the TAA is survivin and the cancer is breast cancer or lung cancer.

In a further particular embodiment, the TAA is cyclin B1, and the cancer is selected from the group consisting of breast, cervical, gastric, colorectal, head and neck squamous cell, non-small-cell lung cancer, colon, prostate, oral and esophageal cancers.

In another particular embodiment, the TAA is HPV, and the type of cancer is cervical cancer.

In a ninth aspect, disclosed herein is a method for treating cancer comprising

administering to a subject in need thereof an effective amount of a recombinant MVA vector expressing a TAA (e.g., MUC-1, survivin, cyclin B1, HPV). According to this embodiment, there is no need to administer TAA to the subject apart from administration of the vector.

Optionally, the method may further comprise administering c) an oncolytic, armed vaccinia virus to the subject.

In a tenth aspect, a method of reducing or preventing growth of a neoplasm in a subject is provided, said method comprising administering to the subject:

a) an effective amount of a recombinant MVA vector expressing a TAA to prime an immune response, and

b) a TAA in an effective amount to boost an immune response to a subject in need thereof to reduce or prevent growth of a neoplasm in the subject, wherein the tumor associated antigen is selected from the group consisting of survivin, cyclin BI, and HPV.

Optionally, the method may further comprise administering c) an oncolytic, armed vaccinia virus to the subject.

In one embodiment, the recombinant MVA vector further comprises one or more additional transgenes. In a particular embodiment, the transgene encodes an immune checkpoint inhibitor, a cytokine or both.

In an eleventh aspect, a method is provided of reducing or preventing growth of a neoplasm in a subject, said method comprising administering to the subject an effective amount of a recombinant MVA vector expressing a TAA (e.g., MUC-1, survivin, cyclin B1, HPV) to prime an immune response. According to this embodiment, there is no need to administer a TAA apart from the vector.

Optionally, the method may further comprise administering c) an oncolytic, armed vaccinia virus to the subject.

In one embodiment, the recombinant MVA vector further comprises one or more additional transgenes. In a particular embodiment, the transgene encodes an immune checkpoint inhibitor, a cytokine or both.

In one embodiment, the subject expresses tumor cell markers, but not yet symptomatic.

In a particular embodiment, treatment results in prevention of a symptomatic disease.

In another embodiment, the subject expresses tumor cell markers but exhibits minimal symptoms of cancer.

In another embodiment, the method results in amelioration of at least one symptom of cancer.

### BRIEF DESCRIPTION OF THE DRAWINGS

FIG. 1 is an electron micrograph showing virus-like particle (VLP) production by cells infected with MVA-MUC-1VP40, an MVA vaccine encoding MUC-1 TAA protein.

FIG. 2 is a western blot demonstrating that cells infected with the MVA-MUC-1 VP40 vaccine (1) express MUC-1 protein, and (2) express hypoglycosylated MUC-1.

FIG. 3 is a graph showing end-point titers of the cross-reactive anti-MUC-1 antibodies assayed with ELISA for sera of non-tumor bearing hMUC-1 transgenic mice immunized with MVA, MTI or a combination of MVA/MTI.

FIG. 4 is a graph showing end-point titers of the cross-reactive anti-MUC-1 antibodies assayed with ELISA for sera of tumor bearing hMUC-1 transgenic mice immunized with MVA, MTI or a combination of MVA/MTI. There is a control group (shown with full circle symbols) from FIG. 3, MVA+MTI, prime/boost from animal of non-tumor bearing hMUC1 transgenic mice.

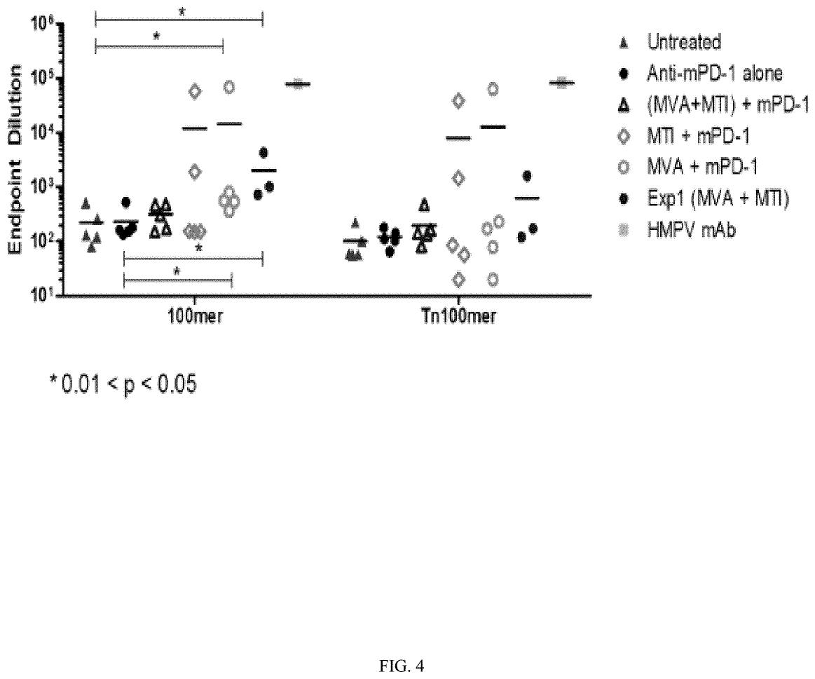

FIG. 5 is a graph showing tumor size progression after administration of tumor cells in a mouse tumor model with various conditions of MVA, MTI, MVA/MTI and anti-mPD-1.

FIG. 6 is a graph showing end tumor weight measurements in a mouse tumor model with various conditions of MVA, MTI, MVA/MTI and anti-mPD-1.

### DETAILED DESCRIPTION OF THE INVENTION

Compositions and methods are provided to produce an immune response to a cancer antigen, such as a tumor associated antigen (TAA) in a subject in need thereof. The compositions and methods disclosed herein can be used to prevent or delay formation of neoplasm or to treat neoplasm or disease associated therewith (such as cancer) in a subject in need thereof. In one embodiment, treatment limits neoplasm development, growth and/or the severity of neoplasm-associated disease such as cancer.

### I. Definitions

Where a term is provided in the singular, the inventors also contemplate aspects of the invention described by the plural of that term. As used in this specification and in the appended claims, the singular forms “a”, “an” and “the” include plural references unless the context clearly dictates otherwise, e.g., “a peptide” includes a plurality of peptides. Thus, for example, a reference to “a method” includes one or more methods, and/or steps of the type described herein, and/or which will become apparent to those persons skilled in the art upon reading this disclosure.

The term “administering” refers to oral administration, topical contact, administration as a suppository, intravenous, intraperitoneal, intramuscular, intralesional, intratumoral, intradermal, intralymphatic, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial.

The term “adjuvant” refers to a substance whose admixture with an administered immunogenic determinant/antigen/nucleic acid construct increases or otherwise modifies the immune response to said determinant. In certain embodiments, no separate adjuvant is required because the viral vector (e.g., MVA) functions as a self-adjuvant.

The term “anti-cancer”, as used herein, refers to an agent capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

The term “antigen” refers to a substance or molecule, such as a protein, or fragment thereof, that is capable of inducing an immune response. The terms “antigen” or “antigenic factors” refer broadly to any antigen to which a human, mammal, bird, or other animal can generate an immune response. The terms “antigen” or “antigenic factors” as used herein refers broadly to a molecule that contains at least one antigenic determinant or epitope to which the immune response may be directed. The immune response may be cell-mediated, humoral or both. As is well known in the art, an antigen may be protein, carbohydrate, lipid, or nucleic acid or any combinations of these biomolecules. As is also well known in the art, an antigen may be native, recombinant, or synthetic. For example, an antigen may include non-natural molecules such as polymers and the like. Antigens include both self-antigens and non-self antigens. As used herein, “antigenic determinant” (or epitope) refers to a single antigenic site on an antigen or antigenic factor; it is a minimal portion of a molecule that recognized by the immune system, specifically by antibodies, B cells or T cells. Antigenic determinants may be linear or discontinuous.

As used herein, the term “antibody” (include, but is not limited, to neutralizing antibodies) refers, for example, to antibodies that block, neutralize, or otherwise act against an infectious foreign antigen.

The term “binding antibody” or “bAb” refers to an antibody which either is purified from, or is present in, a body fluid (e.g., serum or a mucosal secretion) and which recognizes a specific antigen. As used herein, the antibody can be a single antibody or a plurality of antibodies. Binding antibodies include neutralizing and non-neutralizing antibodies.

The term “cancer” refers to a malignant neoplasm that has undergone characteristic anaplasia with loss of differentiation, increase rate of growth, invasion of surrounding tissue, and is capable of metastasis.

The term “cancer antigen” refers to tumor-specific antigens (TSA), tumor-associated antigens (TAA), cells that express tumor-specific antigens, cells that express tumor-associated antigens, embryonic antigens on tumors, autologous tumor cells, tumor-specific membrane antigens, tumor-associated membrane antigens, growth factor receptors, growth factor ligands and any other type of antigen or antigen-presenting cell or material that is associated with a cancer. The cancer vaccines described herein can contain one or more cancer antigens, including but not limited to TAAs.

The term “cell-mediated immune response” refers to the immunological defense provided by lymphocytes, such as the defense provided by sensitized T cell lymphocytes when they directly lyse cells expressing foreign antigens and secrete cytokines (e.g., IFN-gamma.), which can modulate macrophage and natural killer (NK) cell effector functions and augment T cell expansion and differentiation. The cellular immune response is the 2^{nd} branch of the adaptive immune response.

The term “co-administering,” or “co-administration,” refers to the administration of two more agents (e.g., a cancer vaccine and a second therapeutic agent), compounds, therapies, or the like, at or about the same time. The order or sequence of administering the different agents of the disclosed herein may vary and is not confined to any particular sequence. Co-administering may also refer to the situation where two or more agents are administered to different regions of the body or via different delivery schemes, e.g., where a first agent is administered systemically and a second agent is administered intratumorally, or where a first agent is administered intratumorally and a second agent is administering systemically into the blood or proximally to the tumor. Co-administering may also refer to two or more agents administered via the same delivery scheme, e.g., where a first agent is administered intratumorally and a second agent is administered intratumorally.
1. The term “conservative amino acid substitution” refers to substitution of a native amino acid residue with a non-native residue such that there is little or no effect on the size, polarity, charge, hydrophobicity, or hydrophilicity of the amino acid residue at that position, and without resulting in substantially altered immunogenicity. For example, these may be substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Conservative amino acid modifications to the sequence of a polypeptide (and the corresponding modifications to the encoding nucleotides) may produce polypeptides having functional and chemical characteristics similar to those of a parental polypeptide.

The term “control”, as used herein, refers to a reference standard. In some embodiments, the control is a negative control sample obtained from a healthy patient. In other embodiments, the control is a positive control sample obtained from a patient diagnosed with a particular form of cancer. In still other embodiments, the control is a historical control or standard reference value or range of values. A difference between a test sample and a control can be an increase or conversely a decrease. The difference can be a qualitative difference or a quantitative difference, for example, a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

The term “cyclin B1” or “CCNB1” refers to a cyclin B1 protein derived from any mammal, e.g., a human cyclin B1 protein. Human cyclin B1 corresponds to SEQ ID NO:9.

The term “deletion” in the context of a polypeptide or protein refers to removal of codons for one or more amino acid residues from the polypeptide or protein sequence, wherein the regions on either side are joined together. The term deletion in the context of a nucleic acid refers to removal of one or more bases from a nucleic acid sequence, wherein the regions on either side are joined together.

The term “Ebola virus” refers to a virus of species Zaire ebolavirus and has the meaning given to it by the International Committee on Taxonomy of Viruses as documented in (Kuhn, J. H. et al., 2010 Arch. Virol., 155:2083-2103).

The term “Epstein-Barr Virus” or “EBV” as used herein refers to γ1-herpesvirus, one of nine herpesviruses known to infect humans. The host's generation of antigen specific T-cells against viral proteins is very effective against the virus. However, EBV can persist in epithelial or B cells without being completely eliminated, establishing a lifelong persistence in more than 95% of humans.

As used herein, the phrase “an effective amount” in reference to administering the vaccine described herein is an amount that results in an increase in the immune response as measured by an increase in T cell activity or antibody production.

The term “fragment” in the context of a proteinaceous agent refers to a peptide or polypeptide comprising an amino acid sequence of at least 2 contiguous amino acid residues, at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a peptide, polypeptide or protein. In one embodiment the fragment constitutes at least 10%, 20%, 30%, 40%, 50%, 60%, 70%, 80%, or 90% of the entire length of the reference polypeptide. In one embodiment, a fragment of a full-length protein retains activity of the full-length protein. In another embodiment, the fragment of the full-length protein does not retain the activity of the full-length protein.

The term “fragment” in the context of a nucleic acid refers to a nucleic acid comprising an nucleic acid sequence of at least 2 contiguous nucleotides, at least 5 contiguous nucleotides, at least 10 contiguous nucleotides, at least 15 contiguous nucleotides, at least 20 contiguous nucleotides, at least 25 contiguous nucleotides, at least 30 contiguous nucleotides, at least 35 contiguous nucleotides, at least 40 contiguous nucleotides, at least 50 contiguous nucleotides, at least 60 contiguous nucleotides, at least 70 contiguous nucleotides, at least contiguous 80 nucleotides, at least 90 contiguous nucleotides, at least 100 contiguous nucleotides, at least 125 contiguous nucleotides, at least 150 contiguous nucleotides, at least 175 contiguous nucleotides, at least 200 contiguous nucleotides, at least 250 contiguous nucleotides, at least 300 contiguous nucleotides, at least 350 contiguous nucleotides, or at least 380 contiguous nucleotides of the nucleic acid sequence encoding a peptide, polypeptide or protein. In one embodiment the fragment constitutes at least 10%, 20%, 30%, 40%, 50%, 60%, 70%, 80%, or 90% of the entire length of the reference nucleic acid sequence. In a preferred embodiment, a fragment of a nucleic acid encodes a peptide or polypeptide that retains activity of the full-length protein. In another embodiment, the fragment encodes a peptide or polypeptide that of the full-length protein does not retain the activity of the full-length protein.

As used herein, the term “growth inhibitory amount” refers to an amount which inhibits growth or proliferation of a target cell, such as a tumor cell, either in vitro or in vivo, irrespective of the mechanism by which cell growth is inhibited (e.g., by cytostatic properties, cytotoxic properties, etc.). In a preferred embodiment, the growth inhibitory amount inhibits (i.e., slows to some extent and preferably stops) proliferation or growth of the target cell in vivo or in cell culture by greater than about 20%, preferably greater than about 50%, most preferably greater than about 75% (e.g., from about 75% to about 100%).

As used herein, the term “hepatitis B virus” or “HBV” is a partially double-stranded DNA virus, a species of the genus Orthohepadnavirus and a member of the Hepadnaviridae family of viruses. The virus is classified into eight genotypes, A to H. Each genotype has a distinct geographic distribution. Infection with hepatitis B can be acute or chronic.

As used herein, the term “hepatitis C virus” or “HCV” refers to an enveloped positive stranded ribonucleic acid (RNA) virus with a diameter of about 50 nm, belonging to the genus. The RNA virus that lacks a proofreading function, which results in a very high rate of mutations. Rapid mutations in a hypervariable region of the HCV genome coding for the envelope proteins enable the virus to escape immune surveillance by the host. As a consequence, most HCV-infected people proceed to chronic infection.

As used herein, the phrase “heterologous sequence” refers to any nucleic acid, protein, polypeptide, or peptide sequence which is not normally associated in nature with another nucleic acid or protein, polypeptide, or peptide sequence of interest.

As used herein, the phrase “heterologous gene insert” refers to any nucleic acid sequence that has been or is to be inserted into the recombinant vectors described herein. The heterologous gene insert may refer to only the gene product encoding sequence or may refer to a sequence comprising a promoter, a gene product encoding sequence (such as GP, VP or Z), and any regulatory sequences associated or operably linked therewith.

The term “homopolymer stretch” refers to a sequence comprising at least four of the same nucleotides uninterrupted by any other nucleotide, e.g., GGGG or TTTTTTT.

The term “human papilloma virus” or “HPV” refers to double-stranded DNA viruses that infect epithelial cells of the skin and mucosa and in particular, 15 types of HPV that are known to be oncogenic include 16, -18, -31, -33, -35, -39, -45, -51, -52, -56, -58, and -59.

The term “human T-cell lymphotropic virus (HTLV) type I” or “HTLV-I” refers to a retrovirus belonging to the family Retroviridae and the genus Deltaretrovirus. It has a positive-sense RNA genome that is reverse transcribed into DNA and then integrated into the cellular DNA Once integrated, HTLV-1 continues to exist only as a provirus which can spread from cell to cell through a viral synapse.

The term “humoral immune response” refers to the stimulation of Ab production. Humoral immune response also refers to the accessory proteins and events that accompany antibody production, including T helper cell activation and cytokine production, affinity maturation, and memory cell generation. The humoral immune response is one of two branches of the adaptive immune response.

The term “humoral immunity” refers to the immunological defense provided by antibody, such as neutralizing Ab that can directly bind a neoplasm; or, binding Ab that identifies a neoplastic cell for killing by such innate immune responses as complement (C′)-mediated lysis, phagocytosis, and natural killer cells.

The term “immune checkpoint”, as used herein, refers to a component of the immune system which provides inhibitory signals to its components in order to regulate immune reactions. Known immune checkpoint proteins comprise CTLA-4, PD1 and its ligands PD-L1 and PD-L2 and in addition LAG-3, BTLA, B7H3, B7H4, TIM3, KIR. The pathways involving LAGS, BTLA, B7H3, B7H4, TIM3, and KIR are recognized in the art to constitute immune checkpoint pathways similar to the CTLA-4 and PD-1 dependent pathways (see e.g., Pardall, 2012. Nature Rev. Cancer, 12:252-264; Mellman et al., 2011. Nature, 480:480-489). In certain embodiments, an immune checkpoint inhibitor is administered to a subject in need thereof in combination with the immunogenic composition described herein. Inhibition includes reduction of function and full blockade.

The term “immunogenic composition” is a composition that comprises an antigenic molecule where administration of the composition to a subject, results in the development in the subject of a humoral and/or a cellular immune response to the antigenic molecule of interest.

The term “immune response” refers to any response to an antigen or antigenic determinant by the immune system of a subject (e.g., a human). The term “immune response” refers to a cell-mediated (T-cell) immune response and/or an antibody (B-cell) response. Exemplary immune responses include humoral immune responses (e.g., production of antigen-specific antibodies) and cell-mediated immune responses (e.g., production of antigen-specific T cells). Assays for assessing an immune response are known in the art and may comprise in vivo assays, such as assays to measure antibody responses and delayed type hypersensitivity responses. In an embodiment, the assay to measure antibody responses primarily may measure B-cell function as well as B-cell/T-cell interactions. For the antibody response assay, antibody titers in the blood may be compared following an antigenic challenge. As used herein, “antibody titers” can be defined as the highest dilution in post-immune sera that resulted in a value greater than that of pre-immune samples for each subject. The in vitro assays may comprise determining the ability of cells to divide, or to provide help for other cells to divide, or to release lymphokines and other factors, express markers of activation, and lyse target cells. Lymphocytes in mice and man can be compared in in vitro assays. In an embodiment, the lymphocytes from similar sources such as peripheral blood cells, splenocytes, or lymph node cells, are compared. It is possible, however, to compare lymphocytes from different sources as in the non-limiting example of peripheral blood cells in humans and splenocytes in mice. For the in vitro assay, cells may be purified (e.g., B-cells, T-cells, and macrophages) or left in their natural state (e.g., splenocytes or lymph node cells). Purification may be by any method that gives the desired results. The cells can be tested in vitro for their ability to proliferate using mitogens or specific antigens. The ability of cells to divide in the presence of specific antigens can be determined using a mixed lymphocyte reaction (MLR) assay. Supernatant from the cultured cells can be tested to quantitate the ability of the cells to secrete specific lymphokines. The cells can be removed from culture and tested for their ability to express activation antigens. This can be done by any method that is suitable as in the non-limiting example of using antibodies or ligands which bind to the activation antigen as well as probes that bind the RNA coding for the activation antigen.

The term “improved therapeutic outcome” relative to a subject diagnosed as having a neoplasm or cancer refers to a slowing or diminution in the growth of a tumor, or detectable symptoms associated with tumor growth.

The term “inducing an immune response” means eliciting a humoral response (e.g., the production of antibodies) or a cellular response (e.g., the activation of T cells) directed against a cancer antigen in a subject to which the composition (e.g., a vaccine) has been administered.

The term “infectious neoplastic agent” means an infectious agent that can cause a neoplastic disorder (e.g., cancer), such as an oncogenic virus (e.g., human papilloma virus (HPV)).

The term “insertion” in the context of a polypeptide or protein refers to the addition of one or more non-native amino acid residues in the polypeptide or protein sequence. Typically, no more than about from 1 to 6 residues (e.g., 1 to 4 residues) are inserted at any one site within the polypeptide or protein molecule.

The term “Marburg virus” refers to a virus of species Marburg marburgvirus and has the meaning given to it by the International Committee on Taxonomy of Viruses as documented in (Kuhn, J. H. et al. 2010, Arch. Virol., 155:2083-2103).

The term “marker” refers to is meant any substance (e.g., protein or polynucleotide) having an alteration in expression level or activity that is associated with a disease or disorder. A tumor marker is a substance (e.g., protein or polynucleotide) present in, or produced by, cancer cells or other cells in response to cancer or certain benign (non-cancerous) conditions that provide information about the cancer or condition. They can be used along with other tests to establish a cancer diagnosis or monitor treatment.

The term “modified vaccinia Ankara,” “modified vaccinia ankara,” “Modified Vaccinia Ankara,” or “MVA” refers to a highly attenuated strain of vaccinia virus developed by Dr. Anton Mayr by serial passage on chick embryo fibroblast cells; or variants or derivatives thereof. MVA is reviewed in (Mayr, A et al., 1975, Infection, 3:6-14; Swiss Patent No. 568,392).

The term “MTI” as used herein means MUC-1 Tripartite Immunotherapy—a construct having a TLR2 agonist conjugated to a helper epitope conjugated to a MUC-1 epitope for example as described in Lakshminarayanan, V., et al., PNAS, 109(1):261-266 (2012) and shown diagrammatically below.

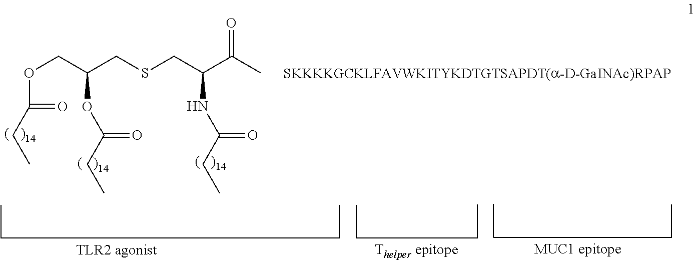

The term “MUC-1 peptide” as used herein means a poly amino acid containing at least 10 consecutive amino acids of the MUC-1 protein sequence. As used herein, MUC-1 peptides include peptide conjugates and hypoglycosylated or non-glycosylated peptides such as for example, but not limited to, MTI and Tn-MUC-1.

The term “neoplasm” as used herein means a new or abnormal growth of tissue in some part of the body especially as a characteristic of cancer.

The term “neutralizing antibody” or “Nab” refers to an antibody which either is purified from, or is present in, a body fluid (e.g., serum or a mucosal secretion) and which recognizes a specific antigen and inhibits the effect(s) of the antigen in the subject (e.g., a human). As used herein, the antibody can be a single antibody or a plurality of antibodies.

The term “non-neutralizing antibody” or “nnAb” refers to a binding antibody that is not a neutralizing antibody.

The term “oncolytic” when used with reference to a virus refers to a virus that preferentially infects and kills cancer cells. The viral infection may lead to tumor regression through two distinct mechanisms: direct killing of tumor cells by replication dependent induced cell death and promotion of an antitumor response towards all tumor cells, including non-infected cells, by inducing immunogenic cell death. As the infected cancer cells are destroyed by oncolysis, they release new infectious virus particles or virions to help destroy the remaining tumor. The oncolytic virus may be any suitable virus. The virus may be derived from human (e.g., herpes simplex virus, adenovirus, measles virus) or animal (e.g., vesicular stomatitis virus (VSV), Newcastle disease virus, myxoma virus). In a particular embodiment, the oncolytic virus is an oncolytic vaccinia virus. The oncolytic virus can be naturally oncolytic or engineered to be oncolytic, i.e., genetically armed to improve or generate more tumor selective cell killing.

Arming can also sensitize the tumor to chemotherapeutic agents or radiotherapy. In a particular embodiment, the oncolytic virus is an armed oncolytic vaccinia virus. The oncolytic virus may have a direct oncolytic effect, an immunomodulatory (e.g., immunostimulatory) effect or both. In a particular embodiment, the oncolytic virus encodes one or more immunomodulatory transgenes, e.g., a cytokine transgene.

The term “operably linked” refers to a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “prevent,” “preventing” and “prevention” refers to the inhibition of the development or onset of a condition (e.g., a tumor or a condition associated therewith), or the prevention of the recurrence, onset, or development of one or more symptoms of a condition in a subject resulting from the administration of a therapy or the administration of a combination of therapies.

The term “prime-boost vaccination”, as used herein, refers to an immunotherapy including administration of a first immunogenic composition (the prime immunogen) followed by administration of a second immunogenic composition (the boost immunogen) to a subject to elicit an immune response. A suitable time interval between administration of the primer vaccine and the booster vaccine can be used. In some embodiments, the prime immunogen, the boost immunogen, or both prime immunogen and the boost immunogen are administered to the subject with an adjuvant to enhance the immune response.

The term “promoter” refers to a polynucleotide sufficient to direct transcription.

The term “prophylactically effective amount” refers to the amount of a composition (e.g., the recombinant MVA vector or pharmaceutical composition) which is sufficient to result in the prevention of the development, recurrence, or onset of a condition or a symptom thereof (e.g., a tumor or a condition or symptom associated therewith or to enhance or improve the prophylactic effect(s) of another therapy).

The term “recombinant” means a polynucleotide of semisynthetic, or synthetic origin that either does not occur in nature or is linked to another polynucleotide in an arrangement not found in nature.

The term “recombinant,” with respect to a viral vector, means a vector (e.g., a viral genome that has been manipulated in vitro, e.g., using recombinant nucleic acid techniques to express heterologous viral nucleic acid sequences).

The term “regulatory sequence” “regulatory sequences” refers collectively to promoter sequences, polyadenylation signals, transcription termination sequences, upstream regulatory domains, origins of replication, internal ribosome entry sites (“IRES”), enhancers, and the like, which collectively provide for the transcription and translation of a coding sequence. Not all of these control sequences need always be present so long as the selected gene is capable of being transcribed and translated.

The term “selectively” means tending to occur at a higher frequency in one population than in another population.

The term “shuttle vector” refers to a genetic vector (e.g., a DNA plasmid) that is useful for transferring genetic material from one host system into another. A shuttle vector can replicate alone (without the presence of any other vector) in at least one host (e.g., E. coli). In the context of MVA vector construction, shuttle vectors are usually DNA plasmids that can be manipulated in E. coli and then introduced into cultured cells infected with MVA vectors, resulting in the generation of new recombinant MVA vectors.

The term “silent mutation” means a change in a nucleotide sequence that does not cause a change in the primary structure of the protein encoded by the nucleotide sequence, e.g., a change from AAA (encoding lysine) to AAG (also encoding lysine).

The term “stage” refers to a classification of the extent of cancer. Factors that are considered when staging a cancer include but are not limited to tumor size, tumor invasion of nearby tissues, and whether the tumor has metastasized to other sites. The specific criteria and parameters for differentiating one stage from another can vary depending on the type of cancer. Cancer staging is used, for example, to assist in determining a prognosis and/or identifying the most appropriate treatment option(s).

The term “subject” means any mammal, including but not limited to, humans, domestic and farm animals, and zoo, sports, or pet animals, such as dogs, horses, cats, cows, rats, mice, guinea pigs and the like. Determination of those subjects “at risk” can be made by any objective or subjective determination by a diagnostic test or opinion of a subject or health care provider (e.g., genetic test, enzyme or protein marker, marker history, and the like).

The term “surrogate endpoint” means a clinical measurement other than a measurement of clinical benefit that is used as a substitute for a measurement of clinical benefit.

The term “surrogate marker” means a laboratory measurement or physical sign that is used in a clinical or animal trial as a substitute for a clinically meaningful endpoint that is a direct measure of how a subject feels, functions, or survives and is expected to predict the effect of the therapy (Katz, R., NeuroRx, 1:189-195 (2004); New drug, antibiotic, and biological drug product regulations; accelerated approval—FDA. Final rule. Fed. Regist., 57: 58942-58960, 1992.).

The term “surrogate marker for protection” means a surrogate marker that is used in a clinical or animal trial as a substitute for the clinically meaningful endpoint of reduction or prevention of neoplasm growth.

The term “synergy” or “synergistic effect” with regard to an effect produced by two or more individual agents refers to a phenomenon in which the total effect produced by these agents when utilized in combination, is greater than the sum of the individual effects of each component acting alone.

The term “synonymous codon” refers to the use of a codon with a different nucleic acid sequence to encode the same amino acid, e.g., AAA and AAG (both of which encode lysine). Codon optimization changes the codons for a protein to the synonymous codons that are most frequently used by a vector or a host cell.

The term “therapeutically effective amount” means the amount of the composition (e.g., the recombinant MVA vector or pharmaceutical composition) that, when administered to a mammal for treating a neoplasm, is sufficient to effect such treatment for the neoplasm.

As used herein, the term “therapeutic immunity” or a “therapeutic immune response” as used herein generally refers to immunity or eliciting an immune response against an infectious agent that ameliorates or eliminates an infection or reduces at least one symptom thereof.

The term “transgene”, as used herein, refers to any gene coding region, either natural or heterologous nucleic acid sequences or fused homologous or heterologous nucleic acid sequences, introduced into the cells or genome of a test subject. In certain aspects, transgenes are carried on any viral vector that is used to introduce the transgenes to the cells of the subject. Recombinant viral vectors can be used to express any TAA disclosed herein including, without limitation, lentiviruses, provirus, vaccinia virus (VV), adenoviruses, adeno-associated viruses, self-complementary adeno-associated virus, cytomegalovirus, Sendai virus, HPV virus, or adenovirus. In some embodiments, the vector is modified vaccinia virus Ankara (MVA).

The term “treating” or “treat” refer to the eradication or control of a neoplasm, the reduction or amelioration of the progression, severity, and/or duration of a condition or one or more symptoms caused by the neoplasm resulting from the administration of one or more therapies.

As used herein, the term “treatment” refers the improvement and/or reversal of the symptoms of disease. A compound which causes an improvement in any parameter associated with disease when used in the screening methods of the instant invention may thereby be identified as a therapeutic compound. The term “treatment” refers to both therapeutic treatment and prophylactic or preventative measures. For example, those who may benefit from treatment with compositions and methods of the present invention include those already with a disease and/or disorder as well as those in which a disease and/or disorder is to be prevented (e.g., using a prophylactic treatment of the present invention).

The term “tumor associated antigen” or “TAA” refers to proteins, glycoproteins, glycolipids, or carbohydrates expressed on the surface of tumor cells. Tumor associated antigen may be, for example, (i) products of mutated oncogenes or tumor suppressor genes or (ii) products of other mutated genes. TAAs may be identified by any suitable method, for example, the “reverse immunology” approach. (Vigneron N, et al., Cancer Immunity. 2013; 13: p. 1).

The term “tumor burden”, as used herein, refers to the number of cancer cells, the size of a tumor, or the amount of cancer in the body. Also called tumor load.

The term “tumor microenvironment” refers to the cellular environment in which any a given tumor exists, including the tumor stroma, surrounding blood vessels, immune cells, fibroblasts, other cells, signaling molecules, and the extracellular membrane.

The term “vaccine” means a substance or composition capable of inducing an immune response in a subject, e.g., a mammal. An immune response against an agent is a humoral, antibody and/or cellular response inducing memory in a subject, resulting in that said agent is being met by a secondary rather than a primary response, thus reducing its impact on the host organism. The term “vaccine” refers to substance which comprises or encodes an antigen that does not induce a disease but provides active immunity against a substance comprising an antigen.

The term “vaccine insert” refers to a nucleic acid sequence encoding a heterologous sequence that is operably linked to a promoter for expression when inserted into a recombinant vector. The heterologous sequence may encode a glycoprotein or matrix protein described here.

The term “vaccinating” refers to therapeutic, or prophylactic or preventative treatment measures.

The term “viral vector” refers to either a nucleic acid molecule (e.g., a plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). Disclosed herein are vaccines for tumor-associated HPV antigens (E2, E6 and E7) to enhance the anti-tumor CD8+ T cell response. The viral vector may vary and include, for example, adenoviral vectors, adeno-associated viral vectors, herpes simplex viral vectors, lentiviral vectors, retroviral vectors, and vaccinia virus (VV) vectors, such as Modified Vaccinia Ankara (MVA). In certain embodiments, the viral vector is replication-deficient in mammalian cells but replication competent in avian cells, e.g., for manufacturing purposes.

The term “virus-like particles” or “VLP” refers to a structure which resembles the native virus antigenically and morphologically.

### II. Immunogenic Compositions

Ideal immunogenic compositions or vaccines have the characteristics of safety, efficacy, scope of protection and longevity, however, compositions having fewer than all of these characteristics may still be useful in preventing neoplasm growth or limiting symptoms or disease progression in an exposed subject treated prior to the development of symptoms. In one embodiment the present invention provides a vaccine that permits at least partial, if not complete, protection after a single immunization.

A. Immunogenic Peptides

The compositions disclosed herein are useful for inducing an immune response to at least one immunogenic peptide. The immunogenic peptide may be any suitable immunogenic peptide(s).

In one embodiment, the immunogenic peptide is a cancer antigen, e.g., a tumor antigen. In certain embodiments, the tumor antigen may be causal or required for maintenance of cellular transformation. In other embodiments, the tumor antigen provides survival or dissemination benefits to developing cancers and are considered as facilitators of disease progression.

In one embodiment, the immunogenic peptide is a tumor associated antigen (TAA).

In a particular embodiment, the TAA is unique, i.e., specific to a tumor and not otherwise found in normal tissue. This is sometimes referred to as a tumor-specific antigen (TSA). The TSA may be, for example, an antigen(s) derived from viral proteins, an antigen(s) derived from point mutations or an antigen(s) encoded by cancer-germline genes.

In certain embodiments, the TAA is a neoantigen, e.g., patient-specific tumor antigen resulted from mutations during oncogenesis.

In a particular embodiment, the TAA is an overexpressed/accumulated TAA. Overexpressed TAAs are expressed on normal cells and tumor cells, but more highly on tumor and/or tumor-associated cells. Representative, non-limiting examples include mucin 1 (MUC-1), human epidermal growth factor receptor 2 (Her2) and human telomerase reverse transcriptase (hTERT).

In another particular embodiment, the TAA is a differentiated TAA. Differentiated TAA's are present on tumor cells and/or tumor-associated cells, as well as certain (less than all) normal cells.

In another particular embodiment, the TAA is an oncofetal TAA. Oncofetal TAA's are present on tumor cells and adult reproductive cells. Representative, non-limiting examples include melanoma antigen E (MAGE), GAGE, BAGE and NY-ESO-1.

The TAA may be a TAA associated with an infectious neoplastic agent, such as an oncogenic virus. Oncogenic viruses are causally linked to between 10 and 20% of all cancers, many of which have mucocutaneous manifestations. Certain oncoviruses can cause various types of cancers, while different oncoviruses can lead to the same disease in diverse mechanisms. Most viral oncogenes are involved in signal transduction, and their expression in infected cells results in constitutive activation of critical pathways that tend to push these cells into the cell cycle. Increased and abnormal proliferation favor the acquisition of additional genetic changes in transformed cells to produce a malignant neoplasm. These changes often result in genetic instability that sets the stage for the acquisition of many additional genetic abnormalities that can eventually allow tumors to invade and metastasize.

The oncogenic virus can be any oncogenic virus (also referred to in the literature as tumor viruses), including a DNA oncogenic virus or an RNA oncogenic virus. Non-limiting examples of oncogenic viruses include human papilloma virus (HPV), hepatitis B virus (HBV), hepatitis C Virus (HCV), Kaposi's Sarcoma-associated herpesvirus (KSHV) (also known as Human Herpes Virus 8), Epstein-Barr Virus (EBV), human polyomaviruses (e.g., Merkel cell polyomavirus (MCPyV), human cytomegalovirus (HCMV) and human T-cell lymphotropic virus type 1 (HTLV-1). The DNA viruses MCPyV, EBV, KSHV, and a subset of oncogenic HPVs are direct carcinogens that encode oncogenes which are required for maintenance of the tumor phenotype.

Neoplasia induced by oncogenic viruses reflect viral cell tropism. For some viruses (e.g., HBV) this is highly restricted, but while others are more widespread (HBV). Nearly all cancers that have been described as caused by oncolytic viruses have increased incidence among immunosuppressed persons. This is particular evident among those oncoviruses that directly transform cells (HTLV-I, HPV, MCV, EBV and KSHV) by expression of foreign oncogenes.

In a particular embodiment, the oncogenic virus is HPV, for example HPV classified as alpha, beta, gamma, mu, and nu HPV. HPV is associated with about 80% of hepatocellular carcinoma. High-risk HPV strains are a major source of cervical cancer and other ano-genital neoplasma, as well as a significant portion of head and neck tumors (e.g., squamous cell head and neck cancer).

In a particular embodiment, the HPV is a high-risk human papillomaviruses (HR-HPV). In certain embodiments, the HPV is HPV 16, -18, -31, -33, -35, -39, -45, -51, -52, -56, -58 or -59. All types of HPV share a common genomic structure and encode eight proteins, including six early proteins: E1, E2, E4, E5, E6 and E7, and two late proteins. E1 and E2 encode proteins implicated in replication and the control of viral transcription, while E5, E6 an E7 are involved in cellular transformation and immortalization.

In a particular embodiment, the virus is HPV and the oncoviral TAA is selected from the E2, E5, E6 and E7 oncoprotein.

In another embodiment, the oncoviral TAA is E2. E2 acts by binding and activating caspase 8 and by the release of cytochrome c from the mitochondria.

In a particular embodiment, the oncoviral TAA is E5. E5 impairs the formation of the death-inducing signaling complex triggered by FasL and TRAIL.

In a particular embodiment, the oncoviral TAA is E6. The E6 protein is a major transforming protein of many types of papillomaviruses. E6 binds to and suppresses p53-mediated apoptosis. It also inhibits Bak, FaDD and procaspase 8e. The high-risk human papillomavirus E6 protein has a unique carboxy terminal PDZ domain containing substrate. In a particular embodiment, the oncoviral TAA is derived from the HPV type 16 E6 (Accession No. NC_001526.2).

In another particular embodiment, the oncoviral TAA is E7. E7 protein acts by binding to retinoblastoma (pRb) as well as to other pocket proteins, such as p107 and p130, leading to the altered activities of these cell cycle regulators. In a particular embodiment, the oncoviral TAA is derived from the HPV type 16 (strain NC_001526).

In one embodiment, the virus is EBV and the oncogenic protein is a EBV nuclear antigen (EBNA) (e.g., EBNA-1) or a latent membrane protein (LMP) (e.g., LMP-1). EBV is associated with nasopharyngeal carcinoma, gastric adenocarcinoma, Hodgkin's lymphoma, Burkitt's lymphoma, large B-cell lymphoma.

In a particular embodiment, the oncoviral TAA is LMP-1.

In a particular embodiment, the oncoviral TAA is EBNA-1. EBNA-1 functions by inhibiting p53-mediated apoptosis.

In another embodiment, the oncovirus is HTLV-1 and the oncogenic protein is Tax or HBZ. Tax is involved in the regulation of cell-cycle, apoptosis, cellular transcription, NFkβ and chromatin remodeling. HTLV-1 is associated with adult T-cell leukemia (ATL).

In another embodiment, the oncovirus is KSHV and the oncogenic protein is latency-associated nuclear antigen LANA (e.g., LANA-1), viral FLICE inhibitory protein (VFlip), vIRF or vGCRP. HTLV-1 is associated with Kaposi's sarcoma, often found in patients with acquired immunodeficiency syndrome (AIDS).

In one embodiment, the virus is HPC and the oncoviral TAA is selected from core, NS3 and NS5A. These oncoviral proteins act by suppressing p53-mediated apoptosis

In another embodiment, the virus is HBV and the oncoprotein is HBx. HBx engages multiple signaling and growth-promoting pathways to induce cell proliferation and enhance ribosome biogenesis.

In a further particular embodiment, the TAA is a tumor-specific, mutated antigen. Representative, non-limiting TAA's of this group include p53.

Other TAAs that can be utilized in the compositions and methods disclosed herein include Wilms' tumor gene (WT1), Tn, TF, and sialyl-Tn (STn) antigens, tumor protein D52 (TPD52), LMP2, EGRFvIII, Her2/Neu, idiotype, MAGE-A3, p3 non-mutant, Mad24, PSMA, GD2, MelanA/Mart1, Ras mutant, gp100, p53 mutant, proteinase 3 (PRI), brc-ab1, tyrosinase, sarcoma translocation breakpoints and the like.

MUC-1

In a particular embodiment, the vectors express MUC-1. In one embodiment, the vectors express a glycosylated form of MUC-1. MUC-1 is found on nearly all epithelial cells, but it is over expressed in cancer cells, and its associated glycans are shorter than those of non-tumor-associated MUC-1 (Gaidzik N, et al., 2013, Chem. Soc. Rev., 42 (10): 4421-42).

The transmembrane glycoprotein Mucin 1 (MUC-1) is aberrantly glycosylated and overexpressed in a variety of epithelial cancers and plays a crucial role in progression of the disease. Tumor-associated MUC-1 differs from the MUC-1 expressed in normal cells with regard to its biochemical features, cellular distribution, and function. In cancer cells, MUC-1 participates in intracellular signal transduction pathways and regulates the expression of its target genes at both the transcriptional and post-transcriptional levels (Nath S., Trends in Mol. Med., Volume 20, Issue 6, p 332-342, June 2014).

In one embodiment, disclosed is a composition comprising a viral vector comprising a sequence encoding MUC-1 and a sequence encoding a matrix protein. In certain embodiments, the viral vector encodes one or more sequences encoding one or more additional transgenes.

In one embodiment, disclosed is a composition comprising a recombinant MVA viral vector expressing MUC-1 and at least one matrix protein that assemble into VLPs.

In various embodiments, immunogenic fragments of MUC-1 may be expressed by the MVA vectors described herein or administered as peptide or peptide fragments to induce or boost an immune response to MUC-1.

In one embodiment, the MUC-1 peptide is an intracellular domain fragment of MUC-1.

In one embodiment, the MUC-1 peptide is an immunogenic intracellular domain fragment of MUC-1 (for example sequence 407-475 of GenBank Protein Accession Number NP_001191214 or an immunogenic fragment thereof).

In one embodiment, the MUC-1 peptide is an immunogenic extracellular domain fragment of MUC-1 (for example sequence 20-376 of GenBank Protein Accession Number NP_001191214 or an immunogenic fragment thereof).

In one embodiment, the MUC-1 peptide comprises the sequence (SEQ ID NO:1): TSAPDTRPAP.

In one embodiment, the MUC-1 peptide comprises MTI.

In one embodiment, the MUC-1 peptide is an extracellular domain fragment of MUC-1 comprising the sequence (SEQ ID NO:2): AHGVTSAPDTRPAPGSTAPP.

In one embodiment, the MUC-1 peptide comprises about 2-10 repeats of a MUC-1 motif AHGVTSAPDTRPAPGSTAPP (SEQ ID NO:2).

In one embodiment, the vectors express an extracellular domain fragment of MUC-1 comprising the sequence (SEQ ID NO:3): AHGVTSAPDNRPALGSTAPP.

In one embodiment, the vectors express an extracellular domain fragment of MUC-1 consisting of the sequence (SEQ ID NO:4): AHGVTSAPDTRPAPGSTAPP AHGVTSAPDNRPALGSTAPP.

In one embodiment, the vectors express an extracellular domain fragment of MUC-1 consisting of the sequence (SEQ ID NO:5): AHGVTSAPDTRPAPGSTAPP AHGVTSAPDTRPAPGSTAPP AHGVTSAPDTRPAPGSTAPP AHGVTSAPDTRPAPGSTAPP AHGVTSAPDNRPALGSTAPP (Tn-100mer).

In one embodiment, the MUC-1 peptide comprises wtMUC-1 GenBank Protein Accession Number NP_001191214 (SEQ ID NO:6). MTPGTQSPFFLLLLLTVLTV VTGSGHASST PGGEKETSAT QRSSVPSSTE KNAVSMTSSV LSSHSPGSGS STTQGQDVTL APATEPASGS AATWGQDVTS VPVTRPALGS TTPPAHDVTS APDNKPAPGS TAPPAHGVTS APDTRPAPGS TAPPAHGVTS APDNRPALGS TAPPVHNVTS ASGSASGSAS TLVHNGTSAR ATTTPASKST PFSIPSHHSD TPTTLASHST KTDASSTHHS TVPPLTSSNH STSPQLSTGV SFFFLSFHIS NLQFNSSLED PSTDYYQELQ RDISEMFLQI YKQGGFLGLS NIKFRPGSVV VQLTLAFREG TINVHDVETQ FNQYKTEAAS RYNLTISDVS VSDVPFPFSA QSGAGVPGWG IALLVLVCVL VALAIVYLIA LAVCQCRRKN YGQLDIFPAR DTYHPMSEYP TYHTHGRYVP PSSTDRSPYE KVSAGNGGSS LSYTNPAVAA TSANL.

In one embodiment, the MUC-1 peptide is included with a TLR2 agonist and T-helper epitope in the sequence (SEQ ID NO:7): SKKKKGCKLFAVWKITYKDTGTSAPDTRPAP wherein the threonine at position 27 is optionally glycosylated with alpha-D-GalNAc.

In certain embodiments, the MUC1 engineered to be displayed on VLPs produced in vivo comprises 5 repeats of the variable number tandem repeat (VNTR) region of MUC1 (SEQ ID NO:3): AHGVTSAPDNRPALGSTAPP.

Survivin

In a particular embodiment, the vectors express survivin. Survivin is expressed in most human neoplasms and embryonic tissues, but is absent or minimal in most normal, differentiated tissues. It is a member of the inhibitor of apoptosis (IAP) family, albeit the smallest at 16.5 kDa and containing 142 amino acids. Survivin functions in cell cycle progression or in apoptosis inhibition depending on its localization and structure state.

Increased levels of survivin effectively inhibit apoptosis, impacting the abnormal proliferation of various cancer cells. Cancers associated with survivin include lung, breast, colon, brain, gastric, esophageal, pancreatic, liver, uterine and ovarian cancer. Increased survivin correlates with poor clinic outcome, tumor recurrence, and therapeutic resistance.

The human sequence of survivin is Uniprot 015392. The full length sequence is shown below (SEQ ID NO:8):

MGAPTLPPAW QPFLKDHRIS TFKNWPFLEG
CACTPERMAEAGFIHCPTEN EPDLAQCFFC
FKELEGWEPD DDPIEEHKKH SSGCAFLSVKKQFEELTLGE
FLKLDRERAK NKIAKETNNK KKEFEETAKK VRRAIEQLAA
MD

In one embodiment, disclosed herein is a composition comprising a viral vector encoding a sequence encoding survivin and a sequence encoding a matrix protein. In certain embodiments, the viral vector further comprises one or more additional sequences encoding one or more additional transgenes.

In a particular embodiment, disclosed herein is a composition comprising MVA encoding a sequence encoding survivin and a sequence encoding a matrix protein.

In a particular embodiment, the vectors express survivin. Survivin is expressed in most human neoplasms and embryonic tissues, but is absent or minimal in most normal, differentiated tissues. It is a member of the inhibitor of apoptosis (IAP) family, albeit the smallest at 16.5 kDa and containing 142 amino acids. Survivin functions in cell cycle progression or in apoptosis inhibition depending on its localization and structure state.

Increased levels of survivin effectively inhibit apoptosis, impacting the abnormal proliferation of various cancer cells. Cancers associated with survivin include lung, breast, colon, brain, gastric, esophageal, pancreatic, liver, uterine and ovarian cancer. Increased survivin correlates with poor clinic outcome, tumor recurrence, and therapeutic resistance.

In one embodiment, the recombinant MVA viral vector expresses survivin and matrix proteins that assemble into VLPs.

In various embodiments, immunogenic fragments of survivin may be expressed by the MVA vectors described herein or administered as peptide or peptide fragments to induce or boost an immune response to survivin.

In one embodiment, the survivin fragment is about five, about 10, about 15, about 20 or about 25 amino acids of survivin.

In a particular embodiment, the survivin fragment is survivin 1-20. In another particular embodiment, the survivin fragment is survivin 95-105.

Cycin B1 Peptide

In a particular embodiment, the vectors express cyclin B1. Cyclin B1 is a protein of 433 amino acids and of 48 kDa, involved in cell cycle regulation and more particularly in the transition from the G2 phase to the M phase. Human cyclin B1 corresponds to the UniProt sequence P14635. The full length sequence is shown below (SEQ ID NO:9):

MALRVTRNSK INAENKAKIN MAGAKRVPTA PAATSKPGLR
PRTALGDIGN KVSEQLQAKM PMKKEAKPSA TGKVIDKKLP
KPLEKVPMLV PVPVSEPVPE PEPEPEPEPV KEEKLSPEPI
LVDTASPSPMETSGCAPAEE DLCQAFSDVI LAVNDVDAED
GADPNLCSEY VKDIYAYLRQ LEEEQAVRPK YLLGREVTGN
MRAILIDWLV QVQMKFRLLQ ETMYMTVSII DRFMQNNCVP
KKMLQLVGVT AMFIASKYEE MYPPEIGDFA FVTDNTYTKH
QIRQMEMKIL RALNFGLGRP LPLHFLRRAS KIGEVDVEQH
TLAKYLMELT MLDYDMVHFP PSQIAAGAFC LALKILDNGE
WTPTLQHYLS YTEESLLPVM QHLAKNVVMV NQGLTKHMTV
KNKYATSKHA KISTLPQLNS ALVQDLAKAV AKV

Cancers associated with cyclin B1 include breast cancer, cervical cancer, lung cancer and melanoma.

In one embodiment, a composition is provided comprising a viral vector comprising sequence encoding cyclin B1 and a matrix protein. In certain embodiments, the viral vector further comprises at least one additional sequence encoding at least one additional transgene.

In one embodiment, the recombinant MVA viral vector expresses cyclin and matrix proteins that assemble into VLPs.

In various embodiments, immunogenic fragments of cyclin may be expressed by the MVA vectors described herein or administered as peptide or peptide fragments to induce or boost an immune response to cyclin.

In one embodiment, the cyclin fragment is about 10, about 20, about 30, about 40, about 50, about 60, about 70, about 80, about 100, about 110, about 120, about 140, about 160, about 180 or about 200 amino acids or more of cyclin B1.

HPV Peptides

In a particular embodiment, the vectors express an HPV peptide.

Of the 60+ types of human papilloma viruses current known, between about 12 and 15 are considered “high-risk”, i.e., oncogenic. These include HPV16, HPV31, HPV33, HPV35, HPV52 and HPV58 (Alphapapillomaviruses-9 species group); HPV18, HPV39, HPV45, HPV59, and HPV68 (Alphapapillomaviruses-7 species group); HPV51 (Alphapapillomaviruses-5 species group); and HPV56 (Alphapapillomaviruses-6 species group). Cancers associated with HPV include vical cancer, anal cancer, head and throat cancers (e.g., head and neck squamous cell carcinoma (HNSCC)).

In a particular embodiment, the HPV peptide is a peptide derived from HPV 16 or HPV 18, or a fragment thereof.

In a particular embodiment, the tumor associated antigen is HPV is E2, E6 or E7.

In one embodiment, a composition is provided comprising a viral vector comprising a sequence encoding an HPV antigen or fragment thereof and a sequence encoding a matrix protein. The viral vector may be any suitable vector, e.g., a vaccine viral vector and more particularly a Modified Vaccinia Ankara vector (MVA). In certain embodiments, the viral vector further comprises one or more additional sequences encoding one or more additional transgenes.

In other aspects, an immunogenic composition is disclosed comprising a) a recombinant modified vaccinia ankara (MVA) viral vector or recombinant vaccinia viral vector comprising a sequence encoding an HPV antigen or fragment thereof and a sequence encoding a matrix protein.

In a particular embodiment, the HPV antigen is selected from the group consisting of HPV antigens E2, E5, E6, E7 or combinations thereof.

In another aspect, a method of inducing an immune response in a subject in need thereof comprising a) administering at least one recombinant MVA vector expressing an HPV antigen or fragment thereof to the subject in an amount sufficient to induce an immune response.

The vaccine platforms can include DNA vaccines and live-vectored, modified vaccinia Ankara (MVA) and live vaccinia virus (VV) vaccine strains. Both viruses are highly safe vaccines that elicit durable CD8+ T cell responses and can be combined in a DNA prime-MVA boost or MVA prime, vaccinia boost regimen to elicit exceptionally high levels of CD8+ T cells. The MVA and vaccinia vaccines are capable of carrying multiple inserts and will easily be able to accommodate E2, E6, and E7 as well as other foreign genes which can be used to modify the form of antigen presentation and the immunostimulatory signals provided by the vector to the responding lymphocytes. Such signals can influence CD8+ effector activity and the ability of elicited cells to traffic into tumors. Initially, 4 different constructs will be made and characterized. Vaccine/s identified as providing the most promising activity will undergo cGMP manufacture and regulatory submissions for a translation clinical trial in HPV-positive Head and Neck patients.

In some embodiments, replication competent vaccinia viruses expressing HPV peptides may be used to induce or boost an immune response to HPV. Vaccinia viruses have also been used to engineer viral vectors for recombinant gene expression and for the potential use as recombinant live vaccines (Mackett, M. et al, 1982, PNAS USA, 79:7415-7419; Smith, G. L., et al., 1984 Biotech. Genet. Engin. Rev., 2:383-407). This entails DNA sequences (genes) which code for foreign antigens being introduced, with the aid of DNA recombination techniques, into the genome of the vaccinia viruses. If the gene is integrated at a site in the viral DNA which is non-essential for the life cycle of the virus, it is possible for the newly produced recombinant vaccinia virus to be infectious, that is to say able to infect foreign cells and thus to express the integrated DNA sequence (EP Patent Applications No. 83,286 and No. 110,385). The recombinant vaccinia viruses prepared in this way can be used, on the one hand, as live vaccines for the prophylaxis of infectious diseases, on the other hand, for the preparation of heterologous proteins in eukaryotic cells.

The immune responses elicited by HPV vaccines are studied for killing and trafficking activity to provide an iterative development pathway for enhancement of vaccine activity. These studies will be done in mice and characterized in patients with head and neck tumors for immune responses to E2, E6, and E7, for the differentiated state of responding CD8+ T cells and their presence in tumors and for the HLA restriction of CD8+ responses to identify anti-E2, E6, E7 responses that are present in patients with head and neck tumors and to determine which of these are associated with control of head and neck tumors.

Clinical samples are provided for immune response analyses and identify a cohort for testing of the vaccines. Vaccines are tested in the presence and absence of checkpoint inhibitors. The primary endpoints for these tests will be the status of anti HPV E2, E6, and E7 CD8+ T cells in patients pre and post-surgery and radiation treatments with and without vaccination. Binding antibodies will also be assayed using standard ELISA.

In one embodiment, the subject is immunized using a protocol where an immune response is primed and boosted with an MVA-VLP-HPV vector, a vaccinia virus, a checkpoint inhibitor, a TLR agonist or combinations thereof.

In a particular embodiment, the checkpoint inhibitor is selected from ipilimumab (Yervoy®), pembrolizumab (Keytruda®), and nivolumab (Opdivo®).

The composition may be administered, e.g., by injection (e.g., intramuscular, intraarterial, intravascular, intravenous, intraperitoneal, or subcutaneous).

It will be appreciated that more than one route of administering the vaccines of the disclosed herein may be employed either simultaneously or sequentially (e.g., boosting). In addition, the vaccines disclosed herein may be employed in combination with traditional immunization approaches such as employing peptide antigens, protein antigens, VLPs, vaccinia virus and inactivated virus, as vaccines. Thus, in one embodiment, the vaccines disclosed herein are administered to a subject (the subject is “primed” with a vaccine disclosed herein) and then a traditional vaccine is administered (the subject is “boosted” with a traditional vaccine).

In another embodiment, a traditional vaccine is first administered to the subject followed by administration of a vaccine disclosed herein. In yet another embodiment, a traditional vaccine and a vaccine disclosed herein are co-administered.

While not to be bound by any specific mechanism, it is believed that upon inoculation with a pharmaceutical composition as described herein, the immune system of the host responds to the vaccine by producing antibodies, both secretory and serum, specific for one or more HPV peptides or immunogenic fragments thereof; and by producing a cell-mediated immune response specific for one or more HPV peptides or immunogenic fragments thereof. As a result of the vaccination, the host becomes at least partially or completely immune to one or more HPV peptides or immunogenic fragments thereof.

It will also be appreciated that single or multiple administrations of the vaccine compositions disclosed herein may be carried out. For example, subjects who are at particularly high risk of recurrence of HPV associated head and neck tumors may require multiple immunizations to establish and/or maintain protective immune responses. Levels of induced immunity can be monitored by measuring amounts of binding and neutralizing secretory and serum antibodies as well as levels of T cells, and dosages adjusted, or vaccinations repeated as necessary to maintain desired levels of protection.

B. Recombinant Viral Vectors Expressing Immunogenic Peptides

Recombinant viral vectors comprising one or more nucleic acid sequences encoding one or more tumor associated antigens or immunogenic fragments thereof, including as disclosed herein, are useful in the methods described herein. Optionally, the recombinant viral vector may encode one or more additional transgenes, for example, a transgene selected from an immune checkpoint inhibitor, a cytokine or an anti-angiogenesis factor.

In certain embodiments, the recombinant viral vector is a vaccinia viral vector, and more particularly, an MVA vector, comprising one or more nucleic acid sequences encoding tumor associated antigen(s) or immunogenic fragments thereof. Examples of such vectors useful in these methods are described in publication WO2017/120577 incorporated by reference herein.

In a particular embodiment, the recombinant viral vector is a vaccine viral vector. In another particular embodiment, the recombinant viral vector is an MVA vector. In a particular embodiment, the vectors express a tumor associated antigen (TAA), including any TAA identified above.

In another embodiment, the vectors express a TAA that is associated with an infectious neoplastic agent.

In one embodiment, the vectors express a glycosylated form of MUC-1. MUC-1 is found on nearly all epithelial cells, but it is over expressed in cancer cells, and its associated glycans are shorter than those of non-tumor-associated MUC-1 (Gaidzik N., et al., 2013, Chem. Soc. Rev., 42 (10): 4421-42).

The transmembrane glycoprotein Mucin 1 (MUC-1) is aberrantly glycosylated and overexpressed in a variety of epithelial cancers and plays a crucial role in progression of the disease. Tumor-associated MUC-1 differs from the MUC-1 expressed in normal cells with regard to its biochemical features, cellular distribution, and function. In cancer cells, MUC-1 participates in intracellular signal transduction pathways and regulates the expression of its target genes at both the transcriptional and post-transcriptional levels (Nath, S., Trends in Mol. Med., Volume 20, Issue 6, p 332-342, June 2014).

In another particular embodiment, the vector expresses survivin or a fragment thereof.

In yet another particular embodiment the vector expresses cyclin B1 or a fragment thereof.

In a still further embodiment, the vector expresses cyclin B1 or a fragment thereof.

In another embodiment, the vector expresses a HBV antigen or a fragment thereof.

In another embodiment, the vector expresses a HPV antigen or a fragment thereof.

In certain embodiments, the vector expresses all immunogenic components of the vaccine and/or it is unnecessary to administer, apart from the vector, a tumor associated antigen (TAA). Rather, the tumor associated antigen is expressed by the vector and sufficient, without administration of soluble antigen, to provide to generate an immune response and/or treat cancer in the subject or reduce or eliminate a neoplasm in the subject.

Several such strains of vaccinia virus have been developed to avoid undesired side effects of smallpox vaccination. Thus, a modified vaccinia Ankara (MVA) has been generated by long-term serial passages of the Ankara strain of vaccinia virus (CVA) on chicken embryo fibroblasts (for review see Mayr, A, et al., 1975, Infection, 3:6-14; Swiss Patent No. 568,392). The MVA virus is publicly available from American Type Culture Collection as ATCC No.: VR-1508. MVA is distinguished by its great attenuation, as demonstrated by diminished virulence, and reduced ability to replicate in primate cells, while maintaining good immunogenicity. The MVA virus has been analyzed to determine alterations in the genome relative to the parental CVA strain. Six major deletions of genomic DNA (deletion I, II, III, IV, V, and VI) totaling 31,000 base pairs have been identified (Meyer, H., et al., 1991 J. Gen. Virol., 72:1031-1038). The resulting MVA virus became severely host cell restricted to avian cells.

Furthermore, MVA is characterized by its extreme attenuation. When tested in a variety of animal models, MVA was proven to be avirulent even in immunosuppressed animals. More importantly, the excellent properties of the MVA strain have been demonstrated in extensive clinical trials (Mayr A., et al., 1978, Zentralbl Bakteriol, [B] 167:375-390; Stickl, et al., 1974 Dtsch. Med. Wschr 99:2386-2392). During these studies in over 120,000 humans, including high-risk patients, no side effects were associated with the use of MVA vaccine.

MVA replication in human cells was found to be blocked late in infection preventing the assembly to mature infectious virions. Nevertheless, MVA was able to express viral and recombinant genes at high levels even in non-permissive cells and was proposed to serve as an efficient and exceptionally safe gene expression vector (Sutter, G. and Moss, B., 1992, PNAS USA, 89:10847-10851). Additionally, novel vaccinia vector vaccines were established on the basis of MVA having foreign DNA sequences inserted at the site of deletion III within the MVA genome (Sutter, G., et al., 1994, Vaccine, 12:1032-1040).

Recombinant MVA vaccinia viruses can be prepared as set out in PCT publication WO2017/120577 incorporated by reference herein. A DNA-construct which contains a DNA-sequence which codes for a foreign polypeptide flanked by MVA DNA sequences adjacent to a predetermined insertion site (e.g., between two conserved essential MVA genes such as I8R/G IL; in restructured and modified deletion III; or at other non-essential sites within the MVA genome) is introduced into cells infected with MVA, to allow homologous recombination. Once the DNA-construct has been introduced into the eukaryotic cell and the foreign DNA has recombined with the viral DNA, it is possible to isolate the desired recombinant vaccinia virus in a manner known per se, preferably with the aid of a marker. The DNA-construct to be inserted can be linear or circular. A plasmid or polymerase chain reaction product is preferred. Such methods of making recombinant MVA vectors are described in PCT publication WO/2006/026667 incorporated by reference herein. The DNA-construct contains sequences flanking the left and the right side of a naturally occurring deletion. The foreign DNA sequence is inserted between the sequences flanking the naturally occurring deletion. For the expression of a DNA sequence or gene, it is necessary for regulatory sequences, which are required for the transcription of the gene, to be present on the DNA Such regulatory sequences (called promoters) are known to those skilled in the art and include for example those of the vaccinia 11 kDa gene as are described in EP-A-198,328, and those of the 7.5 kDa gene (EP-A-110,385). The DNA-construct can be introduced into the MVA infected cells by transfection, for example by means of calcium phosphate precipitation (Graham, et al., 1973 Virol 52:456-467; Wigler, et al., 1979 Cell 16:777-785), by means of electroporation (Neumann, et al., 1982 EMBO J. 1:841-845), by microinjection (Graessmann, et al., 1983, Meth Enzymol 101:482-492), by means of liposomes (Straubinger, et al., 1983, Meth. Enzymol 101:512-527), by means of spheroplasts (Schaffuer, 1980 PNAS USA 77:2163-2167) or by other methods known to those skilled in the art.

The MVA vectors described in WO2017/120577 are immunogenic after a single prime or a homologous prime/boost regimen. Other MVA vector designs require a heterologous prime/boost regimen while still other published studies have been unable to induce effective immune responses with MVA vectors. Conversely, these MVA vector are useful in eliciting effective T-cell and antibody immune responses. Furthermore, the utility of an MVA vaccine vector capable of eliciting effective immune responses and antibody production after a single homologous prime boost is significant for considerations such as use, commercialization and transport of materials especially to affected third world locations.

In one embodiment, disclosed herein is a recombinant viral vector (e.g., an MVA vector) comprising one or more nucleic acid sequences encoding one or more tumor associated antigens (TAAs) disclosed above, e.g., MUC-1, hypoglycosylated MUC-1, survivin, cyclin B-1, HPV, or immunogenic fragments thereof. The viral vector (e.g., an MVA vector) may be constructed using conventional techniques known to one of skill in the art. The one or more heterologous gene inserts encode a polypeptide having desired immunogenicity, i.e., a polypeptide that can induce an immune reaction, cellular immunity and/or humoral immunity, in vivo by administration thereof. The gene region of the viral vector (e.g., an MVA vector) where the gene encoding a polypeptide having immunogenicity is introduced is flanked by regions that are indispensable. In the introduction of a gene encoding a polypeptide having immunogenicity, an appropriate promoter may be operatively linked upstream of the gene encoding a polypeptide having desired immunogenicity.

In some embodiments, replication competent vaccinia viruses expressing the one or more TAAs disclosed above (e.g., MUC-1, survivin, cyclin B1, HPV and the like) may be used to induce or boost an immune response to the TAA. Vaccinia viruses have also been used to engineer viral vectors for recombinant gene expression and for the potential use as recombinant live vaccines (Mackett, M. et al 1982 PNAS USA 79:7415-7419; Smith, G. L. et al., 1984, Biotech Genet Engin Rev 2:383-407). This entails DNA sequences (genes) which code for foreign antigens being introduced, with the aid of DNA recombination techniques, into the genome of the vaccinia viruses. If the gene is integrated at a site in the viral DNA which is non-essential for the life cycle of the virus, it is possible for the newly produced recombinant vaccinia virus to be infectious, that is to say able to infect foreign cells and thus to express the integrated DNA sequence (EP Patent Applications No. 83,286 and No. 110,385). The recombinant vaccinia viruses prepared in this way can be used, on the one hand, as live vaccines for the prophylaxis of infectious diseases, on the other hand, for the preparation of heterologous proteins in eukaryotic cells.

In one embodiment, the nucleic acid sequence encodes an immunogenic extracellular domain sequence of a TAA and a transmembrane domain of the glycoprotein (GP) of Marburgvirus, wherein the TAA is selected from the group consisting of MUC-1, survivin, cyclin B1 and HPV.

In one embodiment, the nucleic acid sequence encodes an immunogenic extracellular domain sequence of MUC-1 and a transmembrane domain of the glycoprotein (GP) of Marburgvirus and an intracellular domain sequence of MUC-1.

In one embodiment, the deletion III site is restructured and modified to remove non-essential flanking sequences.

In exemplary embodiments, the vaccine is constructed to express MUC-1, which is inserted between two conserved essential MVA genes (I8R and G1L) using shuttle vector pGeo-MUC-1; and to express MUC-1, which is inserted into deletion III using shuttle vector pGeo-MUC-1. pGeo-MUC-1 is constructed with an ampicillin resistance marker, allowing the vector to replicate in bacteria; with two flanking sequences, allowing the vector to recombine with a specific location in the MVA genome; with a green fluorescent protein (GFP) selection marker, allowing the selection of recombinant MVAs; with a sequence homologous to part of Flank 1 of the MVA sequence, enabling removal of the GFP sequence from the MVA vector after insertion of MUC-1 into the MVA genome; with a modified H5 (mH5) promoter, which enables transcription of the inserted MUC-1 sequence.

In certain embodiments, the polypeptide, or the nucleic acid sequence encoding the polypeptide, may have a mutation or deletion (e.g., an internal deletion, truncation of the amino- or carboxy-terminus, or a point mutation).

The one or more genes introduced into the recombinant viral vector are under the control of regulatory sequences that direct its expression in a cell. The nucleic acid material of the viral vector may be encapsulated, e.g., in a lipid membrane or by structural proteins (e.g., capsid proteins), that may include one or more viral polypeptides.

In one embodiment, the sequence encoding a MUC-1 peptide or immunogenic fragment thereof is inserted into deletion site I, II, III, IV, V or VI of the MVA vector.

In one embodiment, the sequence encoding a MUC-1 peptide or immunogenic fragment thereof is inserted between I8R and Gil, of the MVA vector, or into restructured and modified deletion III of the MVA vector; and a second sequence encoding a MUC-1 peptide or immunogenic fragment thereof is inserted between I8R and G1L of the MVA vector, or into restructured and modified deletion site III of the MVA vector.

In one embodiment, the recombinant vector comprises in a first deletion site, a nucleic acid sequence encoding a MUC-1 peptide or immunogenic fragment thereof operably linked to a promoter compatible with poxvirus expression systems, and in a second deletion site, a nucleic acid sequence encoding a VLP-forming protein operably linked to a promoter compatible with poxvirus expression systems.

In exemplary embodiments, disclosed herein is a recombinant MVA vector comprising at least one heterologous nucleic acid sequence (e.g., one or more sequences) encoding a MUC-1 peptide or immunogenic fragment thereof which is under the control of regulatory sequences that direct its expression in a cell. The sequence may be, for example, under the control of a promoter selected from the group consisting of Pm2H5, Psyn II, or mH5 promoters.

The recombinant viral vector disclosed herein can be used to infect cells of a subject, which, in turn, promotes the translation into a protein product of the one or more heterologous sequence of the viral vector (e.g., a MUC-1 peptide or immunogenic fragment thereof). As discussed further herein, the recombinant viral vector can be administered to a subject so that it infects one or more cells of the subject, which then promotes expression of the one or more viral genes of the viral vector and stimulates an immune response that is therapeutic or protective against a neoplasm.

In one embodiment, the recombinant MVA vaccine expresses proteins that assemble into virus-like particles (VLPs) comprising the MUC-1 peptide or immunogenic fragment thereof. While not wanting to be bound by any particular theory, it is believed that the MUC-1 peptide is provided to elicit a protective immune response and the matrix protein is provided to enable assembly of VLPs and as a target for T cell immune responses, thereby enhancing the protective immune response and providing cross-protection.

In one embodiment, the matrix protein is a Marburg virus matrix protein.

In one embodiment, the matrix protein is an Ebola virus matrix protein.

In one embodiment, the matrix protein is a Sudan ebolavirus matrix protein.

In one embodiment, the matrix protein is a human immunodeficiency virus type 1 (HIV-1) matrix protein.

In one embodiment, the matrix protein is a human immunodeficiency virus type 1 (HIV-1) matrix protein encoded by the gag gene.

In one embodiment, the matrix protein is a Lassa virus matrix protein.

In one embodiment, the matrix protein is a Lassa virus Z protein.

In one embodiment, the matrix protein is a fragment of a Lassa virus Z protein.

In one embodiment, the matrix protein is a matrix protein of a virus in the Filoviridae virus family.

In one embodiment, the matrix protein is a matrix protein of a virus in the Retroviridae virus family.

In one embodiment, the matrix protein is a matrix protein of a virus in the Arenaviridae virus family.

In one embodiment, the matrix protein is a matrix protein of a virus in the Flaviviridae virus family.

One or more nucleic acid sequences may be optimized for use in an MVA vector. Optimization includes codon optimization, which employs silent mutations to change selected codons from the native sequences into synonymous codons that are optimally expressed by the host-vector system. Other types of optimization include the use of silent mutations to interrupt homopolymer stretches or transcription terminator motifs. Each of these optimization strategies can improve the stability of the gene, improve the stability of the transcript, or improve the level of protein expression from the sequence. In exemplary embodiments, the number of homopolymer stretches in the MUC-1 peptide sequence will be reduced to stabilize the construct. A silent mutation may be provided for anything similar to a vaccinia termination signal. An extra nucleotide may be added in order to express the transmembrane, rather than the secreted, form of any MUC-1 peptide.

In exemplary embodiments, the sequences are codon optimized for expression in MVA; sequences with runs of ≥5 deoxyguanosines, ≥5 deoxycytidines, ≥5 deoxyadenosines, and ≥5 deoxythymidines are interrupted by silent mutation to minimize loss of expression due to frame shift mutations; and the GP sequence is modified through addition of an extra nucleotide to express the transmembrane, rather than the secreted, form of the protein.

Disclosed herein are host cells comprising the recombinant viral vector described above, as well as isolated virions prepared from host cells infected with the recombinant viral vector.

In a particular embodiment, the host cell is an avian cell.

### III. Pharmaceutical Compositions

The recombinant viral vectors or immunogenic peptides described herein are readily formulated as pharmaceutical compositions for veterinary or human use, either alone or in combination. The pharmaceutical composition may comprise a pharmaceutically acceptable diluent, excipient, carrier, or adjuvant.

In one embodiment, disclosed herein is a vaccine effective to protect and/or treat a neoplasm comprising a recombinant MVA vector that expresses at least one TAA (e.g., MUC-1, survivin, cyclin B1, HBV, HPV and the like) or an immunogenic fragment thereof. The vaccine composition may comprise one or more additional therapeutic agents. In certain embodiments, the one or more therapeutic agents are anti-cancer agents.

The pharmaceutical composition may comprise 1, 2, 3, 4 or more than 4 different recombinant viral vectors, e.g., recombinant MVA vectors.

As used herein, the phrase “pharmaceutically acceptable carrier” encompasses any of the standard pharmaceutical carriers, such as those suitable for parenteral administration, such as, for example, by intramuscular, intraarticular (in the joints), intravenous, intradermal, intraperitoneal, and subcutaneous routes. Examples of such formulations include aqueous and non-aqueous, isotonic sterile injection solutions, which contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. One exemplary pharmaceutically acceptable carrier is physiological saline.

Other physiologically acceptable diluents, excipients, carriers, or adjuvants and their formulations are known to those skilled in the art. In one embodiment, adjuvants are used as immune response enhancers. In various embodiments, the immune response enhancer is selected from the group consisting of alum-based adjuvants, oil based adjuvants, Specol, RIBI, TiterMax, Montanide ISA50 or Montanide ISA 720, GM-CSF, nonionic block copolymer-based adjuvants, dimethyl dioctadecyl ammoniumbromide (DDA) based adjuvants AS-1, AS-2, Ribi Adjuvant system based adjuvants, QS21, Quil A, SAF (Syntex adjuvant in its microfluidized form (SAF-m), dimethyl-dioctadecyl ammonium bromide (DDA), human complement based adjuvants m. vaccae, ISCOMS, MF-59, SBAS-2, SBAS-4, Enhanzyn®, RC-529, AGPs, MPL-SE, QS7, Escin; Digitonin; and Gypsophila, Chenopodium quinoa saponins.

The compositions utilized in the methods described herein can be administered by a route selected from, e.g., parenteral, intramuscular, intraarterial, intravascular, intravenous, intraperitoneal, subcutaneous, dermal, transdermal, ocular, inhalation, buccal, sublingual, perilingual, nasal, topical administration, and oral administration. The preferred method of administration can vary depending on various factors (e.g., the components of the composition being administered, and the severity of the condition being treated). Formulations suitable for oral administration may consist of liquid solutions, such as an effective amount of the composition dissolved in a diluent (e.g., water, saline, or PEG-400), capsules, sachets or tablets, each containing a predetermined amount of the vaccine. The pharmaceutical composition may also be an aerosol formulation for inhalation, e.g., to the bronchial passageways. Aerosol formulations may be mixed with pressurized, pharmaceutically acceptable propellants (e.g., dichlorodifluoromethane, propane, or nitrogen).

Pharmaceutical compositions suitable for delivering a therapeutic or biologically active agent can include, e.g., tablets, gel caps, capsules, pills, powders, granulates, suspensions, emulsions, solutions, gels, hydrogels, oral gels, pastes, eye drops, ointments, creams, plasters, drenches, delivery devices, suppositories, enemas, injectables, implants, sprays, or aerosols. Any of these formulations can be prepared by well-known and accepted methods of art. See, for example, Remington: The Science and Practice of Pharmacy (21′ ed.), ed. AR. Gennaro, Lippincott Williams & Wilkins, 2005, and Encyclopedia of Pharmaceutical Technology, ed. J. Swarbrick, Informa Healthcare, 2006, each of which is hereby incorporated by reference.

The immunogenicity of the composition (e.g., vaccine) may be significantly improved if the composition disclosed herein is co-administered with an immunostimulatory agent or adjuvant. Suitable adjuvants well-known to those skilled in the art include, e.g., aluminum phosphate, aluminum hydroxide, QS21, Quil A (and derivatives and components thereof), calcium phosphate, calcium hydroxide, zinc hydroxide, glycolipid analogs, octodecyl esters of an amino acid, muramyl dipeptides, polyphosphazene, lipoproteins, ISCOM-Matrix, DC-Chol, DDA, cytokines, and other adjuvants and derivatives thereof.

Pharmaceutical compositions described herein may be formulated to release the composition immediately upon administration (e.g., targeted delivery) or at any predetermined time period after administration using controlled or extended release formulations. Administration of the pharmaceutical composition in controlled or extended release formulations is useful where the composition, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD50) to median effective dose (ED5o)); (ii) a narrow absorption window in the gastro-intestinal tract; or (iii) a short biological half-life, so that frequent dosing during a day is required in order to sustain a therapeutic level.

Many strategies can be pursued to obtain controlled or extended release in which the rate of release outweighs the rate of metabolism of the pharmaceutical composition. For example, controlled release can be obtained by the appropriate selection of formulation parameters and ingredients, including, e.g., appropriate controlled release compositions and coatings. Suitable formulations are known to those of skill in the art. Examples include single or multiple unit tablet or capsule compositions, oil solutions, suspensions, emulsions, microcapsules, microspheres, nanoparticles, patches, and liposomes.

Formulations suitable for oral administration can consist of (a) liquid solutions, such as an effective amount of the vaccine dissolved in diluents, such as water, saline, or PEG 400; (b) capsules, sachets, or tablets, each containing a predetermined amount of the vaccine, as liquids, solids, granules, or gelatin; (c) suspensions in an appropriate liquid; (d) suitable emulsions; and (e) polysaccharide polymers such as chitins. The vaccine, alone or in combination with other suitable components, may also be made into aerosol formulations to be administered via inhalation, e.g., to the bronchial passageways. Aerosol formulations can be placed into pressurized acceptable propellants, such as dichlorodifluoromethane, propane, nitrogen, and the like.

Suitable formulations for rectal administration include, for example, suppositories, which consist of the vaccine with a suppository base. Suitable suppository bases include natural or synthetic triglycerides or paraffin hydrocarbons. In addition, it is also possible to use gelatin rectal capsules which consist of a combination of the vaccine with a base, including, for example, liquid triglycerides, polyethylene glycols, and paraffin hydrocarbons.

The vaccines disclosed herein may also be co-administered with cytokines to further enhance immunogenicity. The cytokines may be administered by methods known to those skilled in the art, e.g., as a nucleic acid molecule in plasmid form or as a protein or fusion protein.

Also provided herein are kits comprising the vaccines disclosed herein. For example, kits comprising a vaccine and instructions for use are within the scope of this disclosure.

### IV. Method of Use

Disclosed herein are methods of using the immunogenic compositions disclosed herein. The compositions disclosed herein can be used as vaccines for inducing an immune response to a tumor associated antigen (TAA), including, but not limited to, any TAA disclosed herein. In a particular embodiment, the compositions can be used for inducing an immune response to a TAA selected from the group consisting of MUC-1, survivin, cyclin B1, HBV, and/or HPV.

The immune response may be induced in cells isolated from an animal or human subject. In another embodiment, the immune response is induced in a subject at risk for cancer, i.e., a human subject with a family history or cancer or lifestyle-associated risk factors (e.g., smoking). In one embodiment, the subject expresses one or more tumor markers and in certain embodiments, expresses one or more tumor markers but otherwise does not exhibit symptoms typically associated with cancer.

In some embodiments, the subject is naive to cancer treatment, while in others the subject has a cancer that is resistant (has been demonstrated to be resistant) to one or more anti-cancer therapies.

In exemplary embodiments, disclosed herein is a method of inducing an immune response to MUC-1 peptide in a subject in need thereof, said method comprising administering a recombinant viral vector that encodes at least one MUC-1 peptide or immunogenic fragment thereof to the subject in an effective amount to generate an immune response to MUC-1 and a MUC-1 peptide in an effective amount to boost an immune response to MUC-1. The result of the method is that the subject is partially or completely immunized against the MUC-1 peptide.

In one aspect, disclosed is a method of inducing an immune response to a neoplasm in a subject in need thereof, said method comprising:

a) administering a composition comprising an immunogenic vector expressing hypoglycosylated MUC-1 to the subject in an amount sufficient to induce an immune response, or boost a previously induced immune response and

b) administering a composition comprising a MUC-1 peptide in an amount sufficient to induce and immune response or boost a previously induced immune response.

In various embodiments, immunogenic fragments of MUC-1 may be expressed by the MVA vectors described herein or administered as peptide or peptide fragments to induce or boost an immune response to MUC-1.

In one embodiment, the MUC-1 peptide is an intracellular domain fragment of MUC-1.

In one embodiment, the MUC-1 peptide is an immunogenic intracellular domain fragment of MUC-1 (for example sequence 407-475 of GenBank Protein Accession Number NP_001191214 or an immunogenic fragment thereof).

In one embodiment, the MUC-1 peptide is an immunogenic extracellular domain fragment of MUC-1 (for example sequence 20-376 of GenBank Protein Accession Number NP_001191214 or an immunogenic fragment thereof).

In one embodiment, the MUC-1 peptide comprises the sequence TSAPDTRPAP (SEQ ID NO:1) In one embodiment, the MUC-1 peptide comprises MTI.

In one embodiment, the MUC-1 peptide is an extracellular domain fragment of MUC-1 comprising the sequence AHGVTSAPDTRPAPGSTAPP (SEQ ID NO:2).

In one embodiment, the MUC-1 peptide comprises about 2-10 repeats of a MUC-1 motif AHGVTSAPDTRPAPGSTAPP (SEQ ID NO:2).

In one embodiment, the vectors express an extracellular domain fragment of MUC-1 comprising the sequence AHGVTSAPDNRPALGSTAPP (SEQ ID NO:3).

In one embodiment, the vectors express an extracellular domain fragment of MUC-1 consisting of the sequence AHGVTSAPDTRP APGSTAPPAHGVTSAPDNRP ALGSTAPP (SEQ ID NO:4).

In one embodiment, the vectors express an extracellular domain fragment of MUC-1 consisting of the sequence AHGVTSAPDTRPAPGSTAPP AHGVTSAPDTRPAPGSTAPP AHGVTSAPDTRPAPGSTAPP AHGVTSAPDTRPAPGSTAPP AHGVTSAPDNRPALGSTAPP (Tn-100mer) (SEQ ID NO:5).

In one embodiment, the MUC-1 peptide comprises wtMUC-1 GenBank Protein Accession Number NP_001191214 (SEQ ID NO:6).

In one embodiment, the MUC-1 peptide is included with a TLR2 agonist and helper epitope in the sequence SKKKKGCKLFAVWKITYKDTGTSAPDTRPAP (SEQ ID NO:7) wherein the threonine at position 27 is optionally glycosylated with alpha-D-GalNAc.

In one embodiment, the method comprises priming an immune response with an immunogenic vector expressing hypoglycosylated MUC-1 and boosting the immune response with a MUC-1 peptide.

In one embodiment, the immune response is a humoral immune response, a cellular immune response, or a combination thereof.

In a particular embodiment, the immune response comprises production of binding antibodies to MUC-1.

In a particular embodiment, the immune response comprises production of neutralizing antibodies to MUC-1.

In a particular embodiment, the immune response comprises production of non-neutralizing antibodies to MUC-1.

In a particular embodiment, the immune response comprises production of a cell-mediated immune response to MUC-1.

In a particular embodiment, the immune response comprises production of neutralizing and non-neutralizing antibodies to MUC-1.

In a particular embodiment, the immune response comprises production of neutralizing antibodies and cell-mediated immunity to MUC-1.

In a particular embodiment, the immune response comprises production of non-neutralizing antibodies and cell-mediated immunity to MUC-1.

In a particular embodiment, the immune response comprises production of neutralizing antibodies, non-neutralizing antibodies, and cell-mediated immunity to MUC-1.

In one embodiment, the neoplasm is selected from leukemia (e.g., myeloblastic, promyelocytic, myelomonocytic, monocytic, erythroleukemia, chronic myelocytic (granulocytic) leukemia, and chronic lymphocytic leukemia), lymphoma (e.g., Hodgkin's disease and non-Hodgkin's disease), fibrosarcoma, myxosarcoma, liposarcoma, chondrosarcoma, osteogenic sarcoma, angiosarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilms' tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia.

In another embodiment, the neoplasm is selected from adenocarcinomas (breast, colorectal, pancreatic, other), carcinoid tumor, chordoma, choriocarcinoma, desmoplastic small round cell tumor (DSRCT), epithelioid sarcoma, follicular dendritic cell sarcoma, interdigitating dendritic cell/reticulum cell sarcoma, lung: type II pneumocyte lesions (type II cell hyperplasia, dysplastic type II cells, apical alveolar hyperplasia), anaplastic large-cell lymphoma, diffuse large B cell lymphoma (variable), plasmablastic lymphoma, primary effusion lymphoma, epithelioid mesotheliomas, myeloma, plasmacytomas, perineurioma, renal cell carcinoma, synovial sarcoma (epithelial areas), thymic carcinoma (often), Meningioma or Paget's disease.

The compositions disclosed herein can also be used to treat cancer in a subject in need thereof. The particular cancer may express one or more TAAs, including those disclosed above. In a particular embodiment, the cancer expresses MUC-1, survivin, cyclin B2, HBV, or HPV. As such, the MVA vector expresses the particular TAA(s) associated with the cancer in order to generate a therapeutic response.

In a particular embodiment, a method is provided for treating a human papilloma virus-associated cancer, such as an HPV-associated cancer, e.g., cervical cancer, squamous cell head and neck cancer, anal cancer, or vulvar cancer. HPV need not be the sole cause of the HPV-associated cancer, i.e., other factors may contribute.

In another particular embodiment, a method is provided for treating an EBV-associated cancer, e.g., Burkitt's lymphoma, Hodgkin's lymphoma, gastric cancer. EBV need not be the sole cause of the EBY-associated cancer, i.e., other factors may contribute.

In another aspect, disclosed is a method of treating cancer comprising administering:

a) an effective amount of a recombinant MVA vector expressing hypoglycosylated MUC-1 to prime an immune response, and

b) a MUC-1 peptide in an effective amount to boost an immune response to a subject in need thereof to treat cancer.

In another aspect, disclosed is a method of reducing growth of a neoplasm in a subject, said method comprising administering:

a) an effective amount of a recombinant MVA vector expressing hypoglycosylated MUC-1 to prime an immune response, and

b) a MUC-1 peptide in an effective amount to boost an immune response to a subject in need thereof to reduce growth of a neoplasm.

In another aspect, disclosed is a method of reducing or preventing growth of a neoplasm in a subject, said method comprising administering:

a) an effective amount of a recombinant MVA vector expressing hypoglycosylated MUC-1 to prime an immune response, and

b) a MUC-1 peptide in an effective amount to boost an immune response to a subject in need thereof to reduce or prevent growth of a neoplasm in the subject.

In one embodiment, the subject expresses tumor cell markers, but not yet symptomatic. In a particular embodiment, treatment results in prevention of a symptomatic disease.

In another embodiment, the subject expresses tumor cell markers but exhibits minimal symptoms of cancer.

In another embodiment, the method results in amelioration of at least one symptom of cancer.

In one embodiment, methods are disclosed for activating an immune response in a subject using the compositions described herein. In some embodiments, methods are disclosed for promoting an immune response in a subject using a composition described herein. In some embodiments, methods are disclosed for increasing an immune response in a subject using a composition described herein. In some embodiments, methods are disclosed for enhancing an immune response in a subject using a composition described herein.

In exemplary embodiments, disclosed are methods of treating, reducing, preventing, or delaying the growth of a neoplasm in a subject in need thereof, said method comprising administering the composition disclosed herein to the subject in a therapeutically effective amount. The result of treatment is a subject that has an improved therapeutic profile for a disease associated with the neoplasm.

In a particular embodiment, the result of treatment is a reduction in incidence of tumor formation and/or lower tumor burden relative to a control.

In exemplary embodiments, disclosed herein is a method of treating, cancer in a subject in need thereof, said method comprising administering the composition disclosed herein to the subject in a therapeutically effective amount. The result of treatment is a subject that has an improved therapeutic profile for a cancer.

In one embodiment the methods may reduce the growth of the one or more tumors, shrink the one or more tumors, or eradicate the one or more tumors. For example, the tumor mass does not increase. In certain embodiments, the tumor shrinks by 10%, 25%, 50%, 75%, 85%, 90%, 95%, or 99%, or more (or any number therebetween) as compared to its original mass. In certain embodiments, the shrinkage is such that an inoperable tumor is sufficient to permit resection if desired. The concept of substantial shrinkage may also be referred to as “regression,” which refers to a diminution of a bodily growth, such as a tumor. Such a diminution may be determined by a reduction in measured parameters such as, but not limited to, diameter, mass (i.e., weight), or volume. This diminution by no means indicates that the size is completely reduced, only that a measured parameter is quantitatively less than a previous determination.

In one embodiment, the methods may prevent tumor metastasis.

In exemplary embodiments, disclosed herein is a method of treating a proliferative disorder in a subject in need thereof, said method comprising administering the composition of disclosed herein to the subject in a therapeutically effective amount. As used herein, the term “proliferative disorder” refers to a disorder wherein the growth of a population of cells exceeds, and is uncoordinated with, that of the surrounding cells. In certain instances, a proliferative disorder leads to the formation of a tumor. In some embodiments, the tumor is benign, pre-malignant, or malignant. In other embodiments, the proliferative disorder is an autoimmune diseases, vascular occlusion, restenosis, atherosclerosis, or inflammatory bowel disease. In one embodiment, the autoimmune diseases to be treated may be selected from the group consisting of type I autoimmune diseases or type II autoimmune diseases or type III autoimmune diseases or type IV autoimmune diseases, such as, for example, multiple sclerosis (MS), rheumatoid arthritis, diabetes, type I diabetes (Diabetes mellitus), systemic lupus erythematosus (SLE), chronic polyarthritis, Basedow's disease, autoimmune forms of chronic hepatitis, colitis ulcerosa, allergy type I diseases, allergy type II diseases, allergy type III diseases, allergy type IV diseases, fibromyalgia, hair loss, Bechterews disease, Crohn's disease, Myasthenia gravis, neuroclermitis, Polymyalgia rheumatica, progressive systemic sclerosis (PSS), psoriasis, Reiter's syndrome, rheumatic arthritis, psoriasis, vasculitis, etc, or type II diabetes.

In one embodiment, the immune response is a humoral immune response, a cellular immune response or a combination thereof.

In a particular embodiment, the immune response comprises production of binding antibodies against hypoglycosylated MUC-1.

In a particular embodiment, the immune response comprises production of neutralizing antibodies against hypoglycosylated MUC-1.

In a particular embodiment, the immune response comprises production of non-neutralizing antibodies against hypoglycosylated MUC-1.

In a particular embodiment, the immune response comprises production of a cell-mediated immune response against hypoglycosylated MUC-1.

In a particular embodiment, the immune response comprises production of neutralizing and non-neutralizing antibodies against hypoglycosylated MUC-1.

In a particular embodiment, the immune response comprises production of neutralizing antibodies and cell-mediated immunity against hypoglycosylated MUC-1.

In a particular embodiment, the immune response comprises production of non-neutralizing antibodies and cell-mediated immunity against hypoglycosylated MUC-1.

In a particular embodiment, the immune response comprises production of neutralizing antibodies, non-neutralizing antibodies, and cell-mediated immunity against hypoglycosylated MUC-1.

In certain embodiments, the compositions disclosed herein can be used as vaccines for treating a subject at risk of developing a neoplasm, or a subject already having a neoplasm. The recombinant viral vector comprises genes or sequences encoding MUC-1 and viral proteins to promote assembly of virus-like particles (VLPs) or additional enzymes to facilitate expression and glycosylation of hypoglycosylated MUC-1.

In a particular embodiment, the composition comprises a viral vector encoding a sequence encoding MUC-1 (e.g., hypoglycosulated MUC-1) and a matrix protein, optionally further comprising one or more additional transgenes, wherein the composition is administered to treat a subject diagnosed with or at risk of developing a cancer selected from pancreatic cancer, colorectal cancer, prostate cancer, lung cancer, breast cancer or the like.

Typically, the vaccines will be in an admixture and administered simultaneously, but may also be administered separately.

In a particular embodiment, the method may further comprise administering an oncolytic, armed vaccinia virus to the subject in need thereof. This embodiment is generally focused on treatment.

A subject to be treated according to the methods described herein may be one who has been diagnosed by a medical practitioner as having such a condition (e.g., a subject having a neoplasm). Diagnosis may be performed by any suitable means. One skilled in the art will understand that a subject to be treated according method disclosed herein may have been identified using standard tests or may have been identified, without examination, as one at high risk due to the presence of one or more risk factors.

Prophylactic treatment may be administered, for example, to a subject not yet having a neoplasm but who is susceptible to, or otherwise at risk of developing a neoplasm.

Therapeutic treatment may be administered, for example, to a subject already a neoplasm in order to improve or stabilize the subject's condition. The result is an improved therapeutic profile. In some instances, as compared with an equivalent untreated control, treatment may ameliorate a disorder or a symptom thereof by, e.g., 5%, 10%, 20%, 30%, 40%, 50%, 60%, 70%, 80%, 90%, 95%, or 100% as measured by any standard technique.

For example, depending upon the type of cancer, an improved therapeutic profile may be selected from alleviation of one or more symptoms of the cancer, diminishment of extent of disease, stabilized (i.e., not worsening) state of disease, delay or slowing of disease progression, amelioration or palliation of the disease state, remission (whether partial or total), whether detectable or undetectable, tumor regression, inhibition of tumor growth, inhibition of tumor metastasis, reduction in cancer cell number, inhibition of cancer cell infiltration into peripheral organs, improved time to disease progression (TTP), improved response rate (RR), prolonged overall survival (OS), prolonged time-to-next-treatment (TNTT), or prolonged time from first progression to next treatment, or a combination of two or more of the foregoing.

In other embodiments, treatment may result in amelioration of one or more symptoms of a disease associated with a neoplasm (e.g., cancer). According to this embodiment, confirmation of treatment can be assessed by detecting an improvement in or the absence of symptoms.

In one embodiment, disclosed is a method of inducing an immune response in a subject (e.g., a human) by administering to the subject a recombinant viral vector that encodes at least one MUC-1 peptide or immunogenic fragment thereof. The immune response may be a cellular immune response or a humoral immune response, or a combination thereof.

The composition may be administered, e.g., by injection (e.g., intramuscular, intraarterial, intravascular, intravenous, intraperitoneal, or subcutaneous).

It will be appreciated that more than one route of administering the vaccines of the disclosed herein may be employed either simultaneously or sequentially (e.g., boosting). In addition, the vaccines disclosed herein may be employed in combination with traditional immunization approaches such as employing protein antigens, vaccinia virus and inactivated virus, as vaccines. Thus, in one embodiment, the vaccines disclosed herein are administered to a subject (the subject is “primed” with a vaccine disclosed herein) and then a traditional vaccine is administered (the subject is “boosted” with a traditional vaccine). In another embodiment, a traditional vaccine is first administered to the subject followed by administration of a vaccine disclosed herein. In yet another embodiment, a traditional vaccine and a vaccine disclosed herein are co-administered.

While not to be bound by any specific mechanism, it is believed that upon inoculation with a pharmaceutical composition as described herein, the immune system of the host responds to the vaccine by producing antibodies, both secretory and serum, specific for one or more MUC-1 peptides or immunogenic fragments thereof; and by producing a cell-mediated immune response specific for one or more MUC-1 peptides or immunogenic fragments thereof. As a result of the vaccination, the host becomes at least partially or completely immune to one or more MUC-1 peptides or immunogenic fragments thereof, or resistant to developing moderate or severe diseases caused by neoplasm.

In one aspect, methods are provided to alleviate, reduce the severity of, or reduce the occurrence of, one or more of the symptoms associated with a neoplasm comprising administering an effective amount of a pharmaceutical composition comprising a recombinant MVA viral vector that comprises a sequence encoding hypoglycosylated MUC-1, matrix protein sequences, and optionally co-expressing sequences that facilitate expression of and desired glycosylation the MUC-1 peptide.

In another aspect, disclosed herein are methods of providing anti-MUC-1 immunity comprising administering an effective amount of a pharmaceutical composition comprising a recombinant MVA vaccine expressing hypoglycosylated MUC-1 and a viral matrix protein to permit the formation of VLPs.

It will also be appreciated that single or multiple administrations of the vaccine compositions disclosed herein may be carried out. For example, subjects who are at particularly high risk of developing a neoplasm may require multiple immunizations to establish and/or maintain protective immune responses. Levels of induced immunity can be monitored by measuring amounts of binding and neutralizing secretory and serum antibodies as well as levels of T cells, and dosages adjusted, or vaccinations repeated as necessary to maintain desired levels of protection.

In one embodiment, administration is repeated at least twice, at least 3 times, at least 4 times, at least 5 times, at least 6 times, at least 7 times, at least 8 times, or more than 8 times.

In one embodiment, administration is repeated twice.

In one embodiment, about 2-8, about 4-8, or about 6-8 administrations are provided.

In one embodiment, about 1-4 week, 2-4 week, 3-4 week, 1 week, 2 week, 3 week, 4 week or more than 4 week intervals are provided between administrations.

In one specific embodiment, a 4-week interval is used between 2 administrations.

In particular aspect, an immunogenic composition is disclosed comprising a) a recombinant modified vaccinia ankara (MVA) viral vector or recombinant vaccinia viral vector comprising a sequence encoding an HPV antigen or fragment thereof and a matrix protein sequence. In a particular embodiment, the HPV antigens are selected from the group consisting of HPV antigens E2, E5, E6, E7 or combinations thereof. In another aspect, a method of inducing an immune response in a subject in need thereof comprising a) administering at least one recombinant MVA vector expressing an HPV antigen to the subject in an amount sufficient to induce an immune response. In one embodiment, administration is repeated at least twice, at least 3 times, at least 4 times, at least 5 times, at least 6 times, at least 7 times, at least 8 times, or more than 8 times. In one embodiment, administration is repeated twice. In one embodiment, about 2-8, about 4-8, or about 6-8 administrations are provided. In one embodiment, about 1-4 week, 2-4 week, 3-4 week, 1 week, 2 week, 3 week, 4 week or more than 4 week intervals are provided between administrations. In one specific embodiment, a 4-week interval is used between 2 administrations.

In one embodiment, a method is provided of monitoring treatment progress. In exemplary embodiments, the monitoring is focused on biological activity, immune response and/or clinical response.

In one embodiment, the biological activity is a T-cell immune response, regulatory T-cell activity, molecule response (MRD), cytogenic response or conventional tumor response for example, in both the adjuvant or advanced disease setting.

In one embodiment, immune response is monitored for example, by an immune assay such as a cytotoxicity assay, an intracellular cytokine assay, a tetramer assay or an ELISPOT assay.

In one embodiment, clinical response is monitored for example by outcome using established definitions such as response (tumor regression), progression-free, recurrence-free, or overall survival.

In one embodiment, the method includes the step of determining a level of diagnostic marker (e.g., any target delineated herein modulated by a compound herein, a protein or indicator thereof, etc.) or diagnostic measurement (e.g., screen, assay) in a subject having received a therapeutic amount of a compound herein sufficient to treat the disease or symptoms thereof. The level of marker determined in the method can be compared to known levels of marker in either healthy normal controls or in other afflicted patients to establish the subject's disease status. In preferred embodiments, a second level of marker in the subject is determined at a time point later than the determination of the first level, and the two levels are compared to monitor the course of disease or the efficacy of the therapy. In certain preferred embodiments, a pre-treatment level of marker in the subject is determined prior to beginning treatment as disclosed herein; this pre-treatment level of marker can then be compared to the level of marker in the subject after the treatment commences, to determine the efficacy of the treatment.

In one embodiment, upon improvement of a subject's condition (e.g., a change (e.g., decrease) in the level of disease in the subject), a maintenance dose of a compound, composition or combination as disclosed herein may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

In one embodiment, disclosed herein is a method of monitoring treatment progress. In exemplary embodiments, the monitoring is focused on biological activity, immune response and/or clinical response.

In one embodiment, the biological activity is a T-cell immune response, regulatory T-cell activity, molecule response (MRD), cytogenic response or conventional tumor response for example, in both the adjuvant or advanced disease setting.

In one embodiment, immune response is monitored for example, by an immune assay such as a cytotoxicity assay, an intracellular cytokine assay, a tetramer assay or an ELISPOT assay.

In one embodiment, clinical response is monitored for example by outcome using established definitions such as response (tumor regression), progression-free, recurrence free, or overall survival.

In one embodiment, the method includes the step of determining a level of diagnostic marker (e.g., any target delineated herein modulated by a compound herein, a protein or indicator thereof, etc.) or diagnostic measurement (e.g., screen, assay) in a subject having received a therapeutic amount of a compound herein sufficient to treat the disease or symptoms thereof. The level of marker determined in the method can be compared to known levels of marker in either healthy normal controls or in other afflicted patients to establish the subject's disease status. In preferred embodiments, a second level of marker in the subject is determined at a time point later than the determination of the first level, and the two levels are compared to monitor the course of disease or the efficacy of the therapy. In certain preferred embodiments, a pre-treatment level of marker in the subject is determined prior to beginning treatment according to the method disclosed herein; this pre-treatment level of marker can then be compared to the level of marker in the subject after the treatment commences, to determine the efficacy of the treatment.

In one embodiment, upon improvement of a subject's condition (e.g., a change (e.g., decrease)) in the level of disease in the subject), a maintenance dose of a compound, composition or combination as disclosed herein may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

A. Combination with Checkpoint Inhibitors and Chemotherapy

In one embodiment, the above methods can further involve administering a standard of care therapy to the subject. In embodiments, the standard of care therapy is surgery, radiation, radio frequency, cryogenic, ultrasonic ablation, systemic chemotherapy, or a combination thereof.

The vector compositions described herein may be provided as a pharmaceutical composition in combination with other active ingredients. In certain embodiments, the vector composition may be provided in combination with one or more anti-cancer agents.

The active agent may be, without limitation, including but not limited to radionuclides, immunomodulators, anti-angiogenic agents, cytokines, chemokines, growth factors, hormones, drugs, prodrugs, enzymes, oligonucleotides, siRNAs, pro-apoptotic agents, photoactive therapeutic agents, cytotoxic agents, chemotherapeutic agents, toxins, other antibodies or antigen binding fragments thereof.

In a particular embodiment, the immunogenic composition disclosed herein (e.g., MVA-VLP-MUC1) is administered to a subject in need thereof with a checkpoint inhibitor. In certain embodiments, the result is greater efficacy (e.g., in a mouse tumor challenge model, an orthotopic model of PDAC), relative to the immunogenic composition or the checkpoint inhibitor, alone, i.e., the result is synergistic.

In a particular embodiment, the co-administration of the immunogenic composition and the checkpoint inhibitor arrests tumor growth in comparison to a control. In another particular embodiment, the co-administration both arrests tumor growth and reduces tumor volume in comparison to a control. In a particular embodiment, the tumor volume is reduced by about 10%, about 15%, about 20%, about 25%, about 30%, about 35%, about 40%, about 45%, or about 50% or more, in each case in comparison to a control. The control may be untreated or treated with one of the agents, e.g., the immunogenic composition or the checkpoint inhibitor but not both.

In another embodiment, the pharmaceutical composition includes a MUC-1 peptide-expressing vector described herein and a checkpoint inhibitor to activate CD4+, CD8+ effector T-cells to increase tumor clearance.

In various embodiments, the checkpoint inhibitor is an antibody. Antibodies are a key component of the adaptive immune response, playing a central role in both recognizing foreign antigens and stimulating an immune response. Many immunotherapeutic regimens involve antibodies. There are a number of FDA-approved antibodies useful as combination therapies. These antibodies may be selected from Alemtuzumab, Atezolizumab, Ipilimumab, Nivolumab, Ofatumumab, Pembrolizumab, or Rituximab.

Monoclonal antibodies that target either PD-1 or PD-L1 can boost the immune response against cancer cells and have shown a great deal of promise in treating certain cancers. Examples of antibodies that target PD-1 include Pembrolizumab and Nivolumab. An example of an antibody that targets PD-L1 is Atezolizumab.

CTLA-4 is another protein on some T cells that acts as a type of “off switch” to keep the immune system in check. Ipilimumab is a monoclonal antibody that attaches to CTLA-4 to block activity and boost an immune response against a neoplasm.

In another embodiment, the immunogenic vector compositions are administered with adjuvant chemotherapy to increase dendritic cell ability to induce T cell proliferation.

In various embodiments, the vector compositions are administered, before, after or at the same time as chemotherapy.

In certain embodiments, the composition disclosed herein is able to reduce the need of a subject having a tumor or a cancer to receive chemotherapeutic or radiation treatment. In other embodiments, the composition is able to reduce the severity of side effects associated with radiation or chemotherapy in a subject having a tumor or cancer.

The pharmaceutical compositions disclosed herein can be administered alone or in combination with other types of cancer treatment strategies (e.g., radiation therapy, chemotherapy, hormonal therapy, immunotherapy, and anti-tumor agents as described herein. Suitable chemotherapeutic agents useful with these methods include sorafenb, regorafenib, imatinib, eribulin, gemcitabine, capecitabine, pazopani, lapatinib, dabrafenib, sutinib malate, crizotinib, everolimus, torisirolimus, sirolimus, axitinib, gefitinib, anastrole, bicalutamide, fulvestrant, ralitrexed, pemetrexed, goserilin acetate, erlotininb, vemurafenib, visiodegib, tamoxifen citrate, paclitaxel, docetaxel, cabazitaxel, oxaliplatin, ziv-aflibercept, bevacizumab, trastuzumab, pertuzumab, pantiumumab, taxane, bleomycin, melphalen, plumbagin, camptosar, mitomycin-C, mitoxantrone, SMANCS, doxorubicin, pegylated doxorubicin, Folfori, 5-fluorouracil, temozolomide, pasireotide, tegafur, gimeracil, oteraci, itraconazole, bortezomib, lenalidomide, irintotecan, epirubicin, and romidepsin. Preferred chemotherapeutic agents are Carboplatin, Fluorouracil, Vinblastine, Gemcitabine, Cyclophosphamide, Doxorubicin, Methotrexate, Paclitaxel, Topotecan, Etoposide, Methotrexate, Sorafenib, Irinotecan, and Tarceva.

Generic names of cancer chemotherapeutic drugs that have been typically used in cancer patients include: doxorubicin, epirubicin; 5-fluorouracil, paclitaxel, docetaxel, cisplatin, bleomycin, melphalen, plumbagin, irinotecan, mitomycin-C, and mitoxantrone. By way of example, some other cancer chemotherapeutic drugs that may be used and may be in stages of clinical trials include: resminostat, tasquinimod, refametinib, lapatinib, Tyverb, Arenegyr, pasireotide, Signifor, ticilimumab, tremelimumab, lansoprazole, PrevOnco, ABT-869, linifanib, tivantinib, Tarceva, erlotinib, Stivarga, regorafenib, fluoro-sorafenib, brivanib, liposomal doxorubicin, lenvatinib, ramucirumab, peretinoin, Ruchiko, muparfostat, Teysuno, tegafur, gimeracil, oteracil, and orantinib.

Manufacturer brand names for some cancer drugs that may be used in the methods disclosed herein include: NEXAVAR (sorafenb), STIVARGA (regorafenib), AFFINITOR (everolimus), GLEEVEC (imatinib), HALAVEN (eribulin), ALIMTA (pemetrexed), GEMZAR (gemcitabine), VOTRIENT (pazopanib), TYKERB (lapatinib), TAFINIAR (dabrafenib), SUTENT (sutinib malate), XALKORI (crizotinib), TORISEL (torisirolimus), INLYTA (axitinib), IRESSA (gefitinib), ARIMEDEX (anastrole), CASODEX (bicalutamide), FASLODEX (fulvestrant), TOMUDEX (ralitrexed), ZOLADEX (goserilin acetate), TARCEVA (erlotininb), XELODA (capecitabine), ZELBROF (vemurafenib), ERIVEDGE (visiodegib), PERJETA (pertuzumab), HERCEPTIN (trastuzumab), TAXOTERE (docetaxel), JEVTANA (cabazitaxel), ELOXATIN (oxaliplatin), ZALTRAP (ziv-aflibercept), AVASTIN (bevacizumab) Nolvadex, Istubal, and VALODEX (tamoxifen citrate), TEMODAR (temozolomide), SIGNIFOR (pasireotide), VECTIBIX (pantiumumab), ADRIAMYCIN (doxorubicin), DOXIL (pegylated doxorubicin), ABRAXANE (Paclitaxel), TEYSUNO (tegafur, gimeracil, oteracil), BORTEZOMIB (Velcade) and with lenalidomide, ISTODAX (romidepsin).

It is believed that one way that Doxorubicin (ADRIAMYCIN) and DOXIL (pegylated doxorubicin in liposomes) can act to kill cancer cells is by intercalating DNA It is also thought that doxorubicin can become a nitroxide free radical and/or thereby increase cellular levels of free radicals in cancer cells and thereby trigger cellular damage and programmed death. There are potentially serious adverse systemic effects of doxorubicin such as heart damage which limit its use.

5-Fluorouracil (5-FU, Efudex) is a pyrimidine analog which is used in the treatment of cancer. It is a suicide inhibitor and works through irreversible inhibition of thymidylate synthase. Like many anti-cancer drugs, 5-FU's effects are felt system wide but fall most heavily upon rapidly dividing cells that make more frequent use of their nucleotide synthesis machinery, such as cancer cells. 5-FU kills non-cancer cells in parts of the body that are rapidly dividing, for example, the cells lining the digestive tract. Folfori is a treatment with 5-FU, Camptosar, and Irinotecan (leucovorin). The 5-FU incorporates into the DNA molecule and stops synthesis and Camptosar is a topoisomerase inhibitor, which prevents DNA from uncoiling and duplicating. Irinotecan (folinic acid, leucovorin) is a vitamin B derivative used as a “rescue” drug for high doses of the drug methotrexate and that modulates/potentiates/reduces the side effects of the 5-FU (fluorouracil). Mitomycin C is a potent DNA cross-linker. Prolonged use may result in permanent bone-marrow damage. It may also cause lung fibrosis and renal damage.

Taxane agents include paclitaxel (Taxol) and docetaxel (Taxotere). Taxanes disrupt cell microtubule function. Microtubules are essential to cell division. Taxanes stabilize GDP-bound tubulin in the microtubule, thereby inhibiting the process of cell division. Cancer cells can no longer divide. However, taxanes may inhibit cell division of non-cancer cells as well.

Cisplatin(s) which includes carboplatin and oxaliplatin are organic platinum complexes which react in vivo, binding to and causing crosslinking of DNA The cross-linked DNA triggers apoptosis (programmed cell death) of the cancer cells. However, cisplatins can also trigger apoptosis of non-cancer cells. Bleomycin induces DNA strand breaks. Some studies suggest bleomycin also inhibits incorporation of thymidine into DNA strands. Bleomycin will also kill non-cancer cells.

Melphalen (Alkeran) is a nitrogen mustard alkylating agent which adds an alkyl group to the guanine base of DNA Major adverse effects of mephalen include vomiting, oral ulceration, and bone marrow suppression.

Plumbagin has been shown to induce cell cycle arrest and apoptosis in numerous cancer cell lines. It triggers autophagy via inhibition of the Akt/mTOR pathway. It induces G2/M cell cycle arrest and apoptosis through JNK-dependent p53 Ser15 phosphorylation. It promotes autophagic cell death. It inhibits Akt/mTOR signaling. It induces intracellular ROS generation in a PI 5-kinase-dependent manner. To non-cancer cells plumbagin is a toxin, a genotoxin, and a mutagen.

A chemotherapeutic agent may be selected based upon its specificity and potency of inhibition of a cellular pathway target to which cancer cells in the patient may be susceptible. In practicing the disclosed method, the chemotherapeutic agent may be selected by its ability to inhibit a cellular pathway target selected from the group consisting of mTORC, RAF kinase, MEK kinase, Phosphoinositol kinase 3, Fibroblast growth factor receptor, multiple tyrosine kinase, Human epidermal growth factor receptor, vascular endothelial growth factor, other angiogenesis, heat shock protein; Smo (smooth) receptor, FMS-like tyrosine kinase 3 receptor, Apoptosis protein inhibitor, cyclin dependent kinases, deacetylase, ALK tyrosine kinase receptor, serine/threonine-protein kinase Pim-1, Porcupine acyltransferase, hedgehog pathway, protein kinase C, mDM2, Glypciin3, ChK1, Hepatocyte growth factor MET receptor, Epidermal growth factor domain-like 7, Notch pathway, Src-family kinase, DNA methyltransferase, DNA intercalators, Thymidine synthase, Microtubule function disruptor, DNA cross-linkers, DNA strand breakers, DNA alkylators, JNK-dependent p53 Ser15 phosphorylation inducer, DNA topoisomerase inhibitors, Bcl-2, and free radical generators.

In one embodiment, the vector compositions are administered, before, after or at the same time as epigenetic modulators.

In one embodiment, the vector compositions are administered, before, after or at the same time as an epigenetic modulator selected from the group consisting of inhibitors of DNA methyltransferases, inhibitors of histone methyltransferases, inhibitors of histone acetyltransferases, inhibitors of histone deacetylases, and inhibitors of lysine demethylases.

In one embodiment, the vector compositions are administered, before, after or at the same time as an inhibitor of DNA methyltransferases.

In one embodiment, the vector compositions are administered, before, after or at the same time as an inhibitor of histone deacetylases.

In one embodiment, the above methods can further involve administering a checkpoint inhibitor. In one embodiment the checkpoint inhibitor is an antibody or a peptide fragment that interacts with PD1 and PDL1 binding. The antibody sequence of the checkpoint inhibitor or a peptide sequence can be also delivered by MVA or vaccinia vector with or without the HPV antigens such as E6/E7 and E2 or combination of E6/E7 and E2.

In various embodiments, the checkpoint inhibitor is an antibody. Antibodies are a key component of the adaptive immune response, playing a central role in both recognizing foreign antigens and stimulating an immune response. Many immunotherapeutic regimens involve antibodies. There are a number of FDA-approved antibodies useful as combination therapies.

These antibodies may be selected from Alemtuzumab, Atezolizumab, Ipilimumab, Nivolumab, Ofatumumab, Pembrolizumab, or Rituximab.

Monoclonal antibodies that target either PD-1 or PD-L1 can boost the immune response against cancer cells and have shown a great deal of promise in treating certain cancers. Examples of antibodies that target PD-1 include Pembrolizumab and Nivolumab. An example of an antibody that targets PD-L1 is Atezolizumab.

CTLA-4 is another protein on some T cells that acts as a type of “off switch” to keep the immune system in check. Ipilimumab is a monoclonal antibody that attaches to CTLA-4 to block activity and boost an immune response against a neoplasm.

B. Dosage

The vaccines are administered in a manner compatible with the dosage formulation, and in such amount as will be therapeutically effective, immunogenic and protective. The quantity to be administered depends on the subject to be treated, including, for example, the capacity of the immune system of the individual to synthesize antibodies, and, if needed, to produce a cell-mediated immune response. Precise amounts of active ingredient required to be administered depend on the judgment of the practitioner and may be monitored on a patient-by-patient basis. However, suitable dosage ranges are readily determinable by one skilled in the art and generally range from about 5.0×10⁶ TCID₅₀ to about 5.0×10⁹ TCID₅₀. The dosage may also depend, without limitation, on the route of administration, the patient's state of health and weight, and the nature of the formulation.

The pharmaceutical compositions disclosed herein are administered in such an amount as will be therapeutically effective, immunogenic, and/or protective against a neoplasm that expresses a MUC-1 protein or fragment thereof. The dosage administered depends on the subject to be treated (e.g., the manner of administration and the age, body weight, capacity of the immune system, and general health of the subject being treated). The composition is administered in an amount to provide a sufficient level of expression that elicits an immune response without undue adverse physiological effects. Preferably, the composition of the disclosed herein is a heterologous viral vector that includes one MUC-1 peptide or immunogenic fragments thereof and large matrix protein, and is administered at a dosage of, e.g., between 1.0×10⁴ and 9.9×10¹² TCID₅₀ of the viral vector, preferably between 1.0×10⁵ TCID₅₀ and 1.0×10¹¹ TCID₅₀ pfu, more preferably between 1.0×10⁶ and 1.0×10¹⁰ TCID₅₀ pfu, or most preferably between 5.0×10⁶ and 5.0×10⁹ TCID₅₀. The composition may include, e.g., at least 5.0×10⁶ TCID₅₀ of the viral vector (e.g., 1.0×10⁸ TCID₅₀ of the viral vector). A physician or researcher can decide the appropriate amount and dosage regimen.

The composition of the method may include, e.g., between 1.0×10⁴ and 9.9×10¹² TCID₅₀ of the viral vector, preferably between 1.0×10⁵ TCID₅₀ and 1.0×10¹¹ TCID₅₀ pfu, more preferably between 1.0×10⁶ and 1.0×10¹⁰ TCID₅₀ pfu, or most preferably between 5.0×10⁶ and 5.0×10⁹ TCID₅₀. The composition may include, e.g., at least 5.0×10⁶ TCID₅₀ of the viral vector (e.g., 1.0×10⁸ TCID₅₀ of the viral vector). The method may include, e.g., administering the composition to the subject two or more times.

The term “effective amount” is meant the amount of a composition administered to improve, inhibit, or ameliorate a condition of a subject, or a symptom of a disorder, in a clinically relevant manner (e.g., improve, inhibit, or ameliorate disease associated with a neoplasm (e.g., cancer) or provide an effective immune response to a neoplasm). Any improvement in the subject is considered sufficient to achieve treatment. Preferably, an amount sufficient to treat is an amount that prevents the occurrence or one or more symptoms of disease associated with a neoplasm or is an amount that reduces the severity of, or the length of time during which a subject suffers from, one or more symptoms of disease associated with a neoplasm (e.g., by at least 10%, 20%, or 30%, more preferably by at least 50%, 60%, or 70%, and most preferably by at least 80%, 90%, 95%, 99%, or more, relative to a control subject that is not treated with a composition as disclosed herein). A sufficient amount of the pharmaceutical composition used to practice the methods described herein (e.g., the treatment of disease associated with a neoplasm) varies depending upon the manner of administration and the age, body weight, and general health of the subject being treated.

It is important to note that the value of the disclosed compositions and methods may never be demonstrated in terms of actual clinical benefit. Instead, it is likely that the value of the disclosed compositions and methods will be demonstrated in terms of success against a surrogate marker for protection. For an indication such as disease associated with a neoplasm, in which it is impractical or unethical to attempt to measure clinical benefit of an intervention, the FDA's Accelerated Approval process allows approval of a new vaccine based on efficacy against a surrogate endpoint. Therefore, the value of the disclosed compositions and methods may lie in its ability to induce an immune response that constitutes a surrogate marker for protection.

Similarly, FDA may allow approval of vaccines against hypoglycosylated MUC-1 based on its Animal Rule. In this case, approval is achieved based on efficacy in animals.

The composition of the method may include, e.g., between 1.0×10⁴ and 9.9×10¹² TCID₅₀ of the viral vector, preferably between 1.0×10⁵ TCID₅₀ and 1.0×10¹¹ TCID₅₀ pfu, more preferably between 1.0×10⁶ and 1.0×10¹⁰ TCID₅₀ pfu, or most preferably between 5.0×10⁶ and 5.0×10⁹ TCID₅₀. The composition may include, e.g., at least 5.0×10⁶ TCID₅₀ of the viral vector (e.g., 1.0×10⁸ TCID₅₀ of the viral vector). The method may include, e.g., administering the composition two or more times.

In some instances it may be desirable to combine the MUC-1 vaccine disclosed herein with vaccines which induce protective responses to other agents, particularly other MUC-1 peptides. For example, the vaccine compositions disclosed herein can be administered simultaneously, separately or sequentially with other genetic immunization vaccines such as those for influenza (Ulmer, J. B. et al., Science, 259:1745-1749 (1993); Raz, E. et al., PNAS (USA), 91:9519-9523 (1994)), malaria (Doolan, D. L. et al., J. Exp. Med., 183:1739-1746 (1996); Sedegah, M., et al., PNAS (USA) 91:9866-9870 (1994)), and tuberculosis (Tascon, R. C., et al., Nat. Med., 2:888-892 (1996)).

The vaccines are administered in a manner compatible with the dosage formulation, and in such amount as will be therapeutically effective, immunogenic and protective. The quantity to be administered depends on the subject to be treated, including, for example, the capacity of the immune system of the individual to synthesize antibodies, and, if needed, to produce a cell-mediated immune response. Precise amounts of active ingredient required to be administered depend on the judgment of the practitioner and may be monitored on a patient-by-patient basis. However, suitable dosage ranges are readily determinable by one skilled in the art and generally range from about 5.0×10⁶ TCID₅₀ to about 5.0×10⁹ TCID₅₀. The dosage may also depend, without limitation, on the route of administration, the patient's state of health and weight, and the nature of the formulation.

The pharmaceutical compositions disclosed herein are administered in such an amount as will be therapeutically effective, immunogenic, and/or protective against a neoplasm that expresses a HPV protein or fragment thereof. The dosage administered depends on the subject to be treated (e.g., the manner of administration and the age, body weight, capacity of the immune system, and general health of the subject being treated). The composition is administered in an amount to provide a sufficient level of expression that elicits an immune response without undue adverse physiological effects. Preferably, the composition disclosed herein is a heterologous viral vector that includes one HPV peptide or immunogenic fragments thereof and large matrix protein, and is administered at a dosage of, e.g., between 1.0×10⁴ and 9.9×10¹² TCID₅₀ of the viral vector, preferably between 1.0×10⁵ TCID₅₀ and 1.0×10¹¹ TCID₅₀ pfu, more preferably between 1.0×10⁶ and 1.0×10¹⁰ TCID₅₀ pfu, or most preferably between 5.0×10⁶ and 5.0×10⁹ TCID₅₀. The composition may include, e.g., at least 5.0×10⁶ TCID₅₀ of the viral vector (e.g., 1.0×10⁸ TCID₅₀ of the viral vector). A physician or researcher can decide the appropriate amount and dosage regimen.

The composition of the method may include, e.g., between 1.0×10⁴ and 9.9×10¹² TCID₅₀ of the viral vector, preferably between 1.0×10⁵ TCID₅₀ and 1.0×10¹¹ TCID₅₀ pfu, more preferably between 1.0×10⁶ and 1.0×10¹⁰ TCID₅₀ pfu, or most preferably between 5.0×10⁶ and 5.0×10⁹ TCID₅₀. The composition may include, e.g., at least 5.0×10⁶ TCID₅₀ of the viral vector (e.g., 1.0×10⁸ TCID₅₀ of the viral vector). The method may include, e.g., administering the composition to the subject two or more times.

The term “effective amount” is meant the amount of a composition administered to improve, inhibit, or ameliorate a condition of a subject, or a symptom of a disorder, in a clinically relevant manner (e.g., improve, inhibit, or ameliorate disease associated with a neoplasm (e.g. cancer) or provide an effective immune response to a neoplasm). Any improvement in the subject is considered sufficient to achieve treatment. Preferably, an amount sufficient to treat is an amount that prevents the occurrence or one or more symptoms of disease associated with a neoplasm or is an amount that reduces the severity of, or the length of time during which a subject suffers from, one or more symptoms of disease associated with a neoplasm (e.g., by at least 10%, 20%, or 30%, more preferably by at least 50%, 60%, or 70%, and most preferably by at least 80%, 90%, 95%, 99%, or more, relative to a control subject that is not treated with a composition as disclosed herein). A sufficient amount of the pharmaceutical composition used to practice the methods described herein (e.g., the treatment of disease associated with a neoplasm) varies depending upon the manner of administration and the age, body weight, and general health of the subject being treated.

It is important to note that the value of the compositions and methods disclosed herein may never be demonstrated in terms of actual clinical benefit. Instead, it is likely that the value of the compositions and methods disclosed herein will be demonstrated in terms of success against a surrogate marker for protection. For an indication such as disease associated with a neoplasm, in which it is impractical or unethical to attempt to measure clinical benefit of an intervention, the FDA's Accelerated Approval process allows approval of a new vaccine based on efficacy against a surrogate endpoint. Therefore, the value of the disclosed compositions and methods may lie in its ability to induce an immune response that constitutes a surrogate marker for protection.

Similarly, FDA may allow approval of vaccines against HPV based on its Animal Rule. In this case, approval is achieved based on efficacy in animals.

The composition of the method may include, e.g., between 1.0×10⁴ and 9.9×10¹² TCID₅₀ of the viral vector, preferably between 1.0×10⁵ TCID₅₀ and 1.0×10¹¹ TCID₅₀ pfu, more preferably between 1.0×10⁶ and 1.0×10¹⁰ TCID₅₀ pfu, or most preferably between 5.0×10⁶ and 5.0×10⁹ TCID₅₀. The composition may include, e.g., at least 5.0×10⁶ TCID₅₀ of the viral vector (e.g., 1.0×10⁸ TCID₅₀ of the viral vector). The method may include, e.g., administering the composition two or more times.

In some instances, it may be desirable to combine the HPV vaccine disclosed herein with vaccines which induce protective responses to other agents, particularly other HPV peptides. For example, the vaccine compositions disclosed herein can be administered simultaneously, separately or sequentially with other genetic immunization vaccines such as those for influenza (Ulmer, J. B. et al., Science 259: 1745-1749 (1993); Raz, E. et al., PNAS (USA) 91:9519-9523 (1994)), malaria (Doolan, D. L. et al., J. Exp. Med. 183: 1739-1746 (1996); Sedegah, M. et al., PNAS (USA) 91:9866-9870 (1994)), and tuberculosis (Tascon, R. C. et al., Nat. Med. 888-892 (1996)).

C. Administration

As used herein, the term “administering” refers to a method of giving a dosage of a pharmaceutical composition disclosed herein to a subject. The compositions utilized in the methods described herein can be administered by a route selected from, e.g., parenteral, dermal, transdermal, ocular, inhalation, buccal, sublingual, perilingual, nasal, rectal, topical administration, and oral administration. Parenteral administration includes intravenous, intraperitoneal, subcutaneous, intraarterial, intravascular, and intramuscular administration. The preferred method of administration can vary depending on various factors (e.g., the components of the composition being administered, and the severity of the condition being treated).

Administration of the pharmaceutical compositions (e.g., vaccines) disclosed herein can be by any of the routes known to one of skill in the art. Administration may be by, e.g., intramuscular injection. The compositions utilized in the methods described herein can also be administered by a route selected from, e.g., parenteral, dermal, transdermal, ocular, inhalation, buccal, sublingual, perilingual, nasal, rectal, topical administration, and oral administration. Parenteral administration includes intravenous, intraperitoneal, subcutaneous, and intramuscular administration. The preferred method of administration can vary depending on various factors, e.g., the components of the composition being administered, and the severity of the condition being treated.

In addition, single or multiple administrations of the compositions disclosed herein may be given to a subject. For example, subjects who are particularly susceptible to developing a neoplasm may require multiple treatments to establish and/or maintain protection against the neoplasm. Levels of induced immunity provided by the pharmaceutical compositions described herein can be monitored by, e.g., measuring amounts of neutralizing secretory and serum antibodies. The dosages may then be adjusted or repeated as necessary to maintain desired levels of protection against development of a neoplasm or to reduce growth of a neoplasm.

Increased vaccination efficacy can be obtained by timing the administration of the vector. Any of the priming and boosting compositions described above are suitable for use with the methods described here.

In one embodiment, recombinant MUC-1 expressing MVA vectors are administered to prime an immune response and the primed immune response is boosted at a time after the first MVA administration.

In one embodiment, a MUC-1-expressing MVA vector is administered to prime an immune response and a composition comprising a MUC-1-expressing MVA, a MUC-1 peptide, and/or a checkpoint inhibitor is administered to boost the immune response primed with the MUC-1-expressing MVA vector.

In one embodiment, MVA vectors are used for both priming and boosting purposes. Such protocols include but are not limited to MM, MAIM, and MMMM.

In some embodiments, one, two, three, four, five, six, seven, eight, nine, ten or more than ten MVA or MUC-1 peptide boosts are administered.

In some embodiments, one, two, three, four, five, six, seven, eight, nine, ten or more than ten doses of checkpoint inhibitor are administered.

Vectors can be administered alone (i.e., a plasmid can be administered on one or several occasions with or without an alternative type of vaccine formulation (e.g., with or without administration of protein or another type of vector, such as a viral vector)) and, optionally, with an adjuvant or in conjunction with (e.g., prior to) an alternative booster immunization (e.g., a live-vectored vaccine such as a recombinant modified vaccinia Ankara vector (MVA)) comprising an insert that may be distinct from that of the “prime” portion of the immunization or may be a related vaccine insert(s). For example, GM-CSF or other adjuvants known to those of skill in the art. The adjuvant can be a “genetic adjuvant” (i.e., a protein delivered by way of a DNA sequence).

In exemplary embodiments, the method disclosed herein is an immunization method comprising (i) administering a priming composition comprising a MVA vector comprising one or more sequences encoding a hypoglycosylated MUC-1 or immunogenic fragment thereof; (ii) administering a first dose of a boosting composition comprising a hypoglycosylated MUC-1 peptide or immunogenic fragment thereof.

In a particular embodiment, the hypoglycosylated MUC-1 peptides are the same in step (i)-(iii). Optionally, the method further comprises one or more additional steps, including, for example, the administration of one or more additional doses of the priming composition or a different priming composition (i.e., a second priming composition) and/or one or more additional doses of the boosting composition or a different boosting composition (i.e., a second boosting composition).

D. Indications

In specific embodiments, the immunogenic vectors useful in the present methods may be administered to a subject with a neoplasm or a subject diagnosed with prostate, breast, lung, liver, endometrial, bladder, colon or cervical carcinoma; adenocarcinoma; melanoma; lymphoma; glioma; or sarcomas such as soft tissue and bone sarcomas.

In a further embodiment the method disclosed herein utilizes the compositions disclosed herein for the treatment or prevention of cancer, including, but not limited to, neoplasms, tumors, metastases, or any disease or disorder characterized by uncontrolled cell growth, and particularly multidrug resistant forms thereof. The cancer can be a multifocal tumor. Examples of types of cancer and proliferative disorders to be treated with the therapeutics disclosed herein include, but are not limited to, leukemia (e.g., myeloblastic, promyelocytic, myelomonocytic, monocytic, erythroleukemia, chronic myelocytic (granulocytic) leukemia, and chronic lymphocytic leukemia), lymphoma (e.g., Hodgkin's disease and non-Hodgkin's disease), fibrosarcoma, myxosarcoma, liposarcoma, chondrosarcoma, osteogenic sarcoma, angiosarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilms' tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia. In a particular embodiment, therapeutic compositions disclosed herein are administered to patients having prostate cancer (e.g., prostatitis, benign prostatic hypertrophy, benign prostatic hyperplasia (BPH), prostatic paraganglioma, prostate adenocarcinoma, prostatic intraepithelial neoplasia, prostato-rectal fistulas, and atypical prostatic stromal lesions). In an especially preferred embodiment, the medicaments disclosed herein are used for the treatment of cancer, glioma, liver carcinoma and/or colon carcinoma. The treatment and/or prevention of cancer includes, but is not limited to, alleviating symptoms associated with cancer, the inhibition of the progression of cancer, the promotion of the regression of cancer, and the promotion of the immune response.

As used herein, the term neoplasm refers to an abnormal growth of tissue. A neoplasm may be benign or malignant. Generally, a malignant neoplasm is referred to as a cancer. Cancers differ from benign neoplasms in the ability of malignant cells to invade other tissues, either by direct growth into adjacent tissue through invasion or by implantation into distant sites by metastasis (i.e., transport through the blood or lymphatic system). The methods disclosed herein are suitable for the treatment of benign and malignant neoplasms (cancer).

As defined herein a superficial neoplasm is one located on the outer surface of the body that has confined itself and not spread to surrounding tissues or other parts of the body. An internal neoplasms located on an internal organ or other internal part of the body. An invasive neoplasm is a neoplasm that has started to break through normal tissue barriers and invade surrounding areas, e.g., an invasive breast cancer that has spread beyond the ducts and lobules.

A non-exclusive list of the types of neoplasms contemplated for treatment by the method disclosed herein includes the following categories: (a) abdominal neoplasms including peritoneal neoplasms and retroperitoneal neoplasms; (b) bone neoplasms including femoral neoplasms, skull neoplasms, jaw neoplasms, manibular neoplasms, maxillary neoplasms, palatal neoplasms, nose neoplasms, orbital neoplasms, skull base neoplasms, and spinal neoplasms; c) breast neoplasms including male breast neoplasms, breast ductal carcinoma, and phyllodes tumor; (d) digestive system neoplasms including biliary tract neoplasms, bile duct neoplasms, common bile duct neoplasms, gall bladder neoplasms, gastrointestinal neoplasms, esophegeal neoplasms, intestinal neoplasms, cecal neoplasms, appendiceal neoplasms, colorectal neoplasms, colorectal adenomatous polyposis coli, colorectal Gardner Syndrome, colonic neoplasms, colonic adenomatous polyposis coli, colonic Gardner Syndrome, sigmoid neoplasms, hereditary nonpolyposis colorectal neoplasms, rectal neoplasms, anus neoplasms, duodenal neoplasms, ileal neoplasms, jejunal neoplasms, stomach neoplasms, liver neoplasms, liver cell adenoma, hepatocellular carcinoma, pancreatic neoplasms, islet cell adenoma, insulinoma, islet cell carcinoma, gastrinoma, glucagonoma, somatostatinoma, vipoma, pancreatic ductal carcinoma, and peritoneal neoplasms; (e) endocrine gland neoplasms including adrenal gland neoplasms, adrenal cortex neoplasms, adrenocortical adenoma, adrenocortical carcinoma, multiple endocrine neoplasia, multiple endocrine neoplasia type 1, multiple endocrine neoplasia type 2a, multiple endocrine neoplasia type 2b, ovarian neoplasms, granulosa cell tumor, luteoma, Meigs' Syndrome, ovarian Sertoli-Leydig cell tumor, thecoma, pancreatic neoplasms, paraneoplastic endocrine syndromes, parathyroid neoplasms, pituitary neoplasms, Nelson Syndrome, testicular neoplasms, testicular Sertoli-Leydig cell tumor, and thyroid neoplasms (f) eye neoplasms including conjunctival neoplasms, orbital neoplasms, retinal neoplasms, retinoblastoma, uveal neoplasms, choroid neoplasms, and iris neoplasms; (g) brain, head and neck neoplasms including esophageal neoplasms, facial neoplasms, eyelid neoplasms, mouth neoplasms, gingival neoplasms, oral leukoplakia, hairy leukoplakia, lip neoplasms, palatal neoplasms, salivary gland neoplasms, parotid neoplasms, sublingual gland neoplasms, submandibular gland neoplasms, tongue neoplasms, otorhinolaryngologic neoplasms, ear neoplasms, laryngeal neoplasms, nose neoplasms, paranasal sinus neoplasms, maxillary sinus neoplasms, pharyngeal neoplasms, hypopharyngeal neoplasms, nasopharyngeal neoplasms, nasopharyngeal neoplasms, oropharyngeal neoplasms, tonsillar neoplasms, parathyroid neoplasms, thyroid neoplasms, and tracheal neoplasms; (h) hematologic neoplasms including bone marrow neoplasms; (i) nervous system neoplasms including central nervous system neoplasms, brain neoplasms, cerebral ventricle neoplasms, choroid plexus neoplasms, choroid plexus papilloma, infratentorial neoplasms, brain stem neoplasms, cerebellar neoplasms, neurocytoma, pinealoma, supratentorial neoplasms, hypothalamic neoplasms, pituitary neoplasms, Nelson Syndrome, cranial nerve neoplasms, optic nerve neoplasms, optic nerve glioma, acoustic neuroma, neurofibromatosis 2, nervous system paraneoplastic syndromes, Lambert-Eaton myasthenic syndrome, limbic encaphalitis, transverse myelitis, paraneoplastic cerebellar degeneration, paraneoplastic polyneuropathy, peripheral nervous system neoplasms, cranial nerve neoplasms, acoustic neuroma, and optic nerve neoplasms; G) pelvic neoplasms; (k) skin neoplasms including acanthoma, sebaceous gland neoplasms, sweat gland neoplasms and basal cell carcinoma; (1) soft tissue neoplasms including muscle neoplasms and vascular neoplasms; (m) splenic neoplasms; (n) thoracic neoplasms including heart neoplasms, mediastinal neoplasms, respiratory tract neoplasms, bronchial neoplasms, lung neoplasms, bronchogenic carcinoma, non-small-cell lung carcinoma, pulmonary coin lesion, Pancoasts's Syndrome, pulmonary blastoma, pulmonary sclerosing hemangioma, pleural neoplasms, malignant pleural effusion, tracheal neoplasms, thymus neoplasms, and thymoma; (o) urogenital neoplasms including female genital neoplasms, fallopian tube neoplasms, uterine neoplasms, cervix neoplasms, endometrial neoplasms, endometrioid carcinoma, endometrial stromal tumors, endometrial stromal sarcoma, vaginal neoplasms, vulvar neoplasms, male genital neoplasms, penile neoplasms, prostatic neoplasms, testicular neoplasms, urologic neoplasms, bladder neoplasms, kidney neoplasms, renal cell carcinoma, nephroblastoma, Denys-Drash Syndrome, WAGR Syndrome, mesoblastic nephroma, ureteral neoplasms and urethral neoplasms; (p) and additional cancers including renal carcinoma, lung cancer, melanoma, leukemia, Barrett's esophagus, metaplasia pre-cancer cells.

In one embodiment, the immune response stimulating vectors described herein express MUC-1 or an immunogenic fragment thereof and are particularly useful for treating Adenocarcinomas (breast, colorectal, pancreatic, other), Carcinoid tumor, Chordoma, Choriocarcinoma, Desmoplastic small round cell tumor (DSRCT), Epithelioid sarcoma, Follicular dendritic cell sarcoma, interdigitating dendritic cell/reticulum cell sarcoma, Lung: type II pneumocyte lesions (type II cell hyperplasia, dysplastic type II cells, apical alveolar hyperplasia), Anaplastic large-cell lymphoma, diffuse large B cell lymphoma (variable), plasmablastic lymphoma, primary effusion lymphoma, Epithelioid mesotheliomas, Myeloma, Plasmacytomas, Perineurioma, Renal cell carcinoma, Synovial sarcoma (epithelial areas), Thymic carcinoma (often), Meningioma or Paget's disease.

The compositions and methods disclosed herein are further described by way of the following non-limiting examples. Further aspects and embodiments of the compositions and methods disclosed herein will be apparent to those of ordinary skill in the art, in view of the above disclosure and following experimental exemplification, included by way of illustration and not limitation, and with reference to the attached figures.

Administration of the pharmaceutical compositions (e.g., vaccines) disclosed herein can be by any of the routes known to one of skill in the art. Administration may be by, e.g., intramuscular injection. The compositions utilized in the methods described herein can also be administered by a route selected from, e.g., parenteral, dermal, transdermal, ocular, inhalation, buccal, sublingual, perilingual, nasal, rectal, topical administration, and oral administration. Parenteral administration includes intravenous, intraperitoneal, subcutaneous, and intramuscular administration. The preferred method of administration can vary depending on various factors, e.g., the components of the composition being administered, and the severity of the condition being treated.

In addition, single or multiple administrations of the compositions disclosed herein may be given to a subject. For example, subjects who are particularly susceptible to developing a neoplasm may require multiple treatments to establish and/or maintain protection against the neoplasm. Levels of induced immunity provided by the pharmaceutical compositions described herein can be monitored by, e.g., measuring amounts of neutralizing secretory and serum antibodies. The dosages may then be adjusted or repeated as necessary to maintain desired levels of protection against development of a neoplasm or to reduce growth of a neoplasm.

Increased vaccination efficacy can be obtained by timing the administration of the vector. Any of the priming and boosting compositions described above are suitable for use with the methods described here.

In one embodiment, recombinant HPV-expressing MVA vectors are administered to prime an immune response and the primed immune response is boosted at a time after the first MVA administration.

In one embodiment, a HPV-expressing MVA vector is administered to prime an immune response and a composition comprising a HPV-expressing MVA, vaccinia, a HPV peptide, and/or a checkpoint inhibitor is administered to boost the immune response primed with the HPV-expressing MVA vector.

In one embodiment, MVA vectors are used for both priming and boosting purposes. Such protocols include but are not limited to MM, MMM, and MMMM.

In another embodiment MVA vectors are used for priming and vaccinia vectors are used for boosting. Priming and boosting can be combined with coadministration of checkpoint inhibitors (as separate or integrated into the vectors) and peptide/protein antigens, VLPs with or without adjuvants.

In some embodiments, one, two, three, four, five, six, seven, eight, nine, ten, or more than ten MVA or HPV peptide boosts are administered.

In some embodiments, one, two, three, four, five, six, seven, eight, nine, ten, or more than ten doses of checkpoint inhibitor are administered.

Vectors can be administered alone (i.e., a plasmid can be administered on one or several occasions with or without an alternative type of vaccine formulation (e.g., with or without administration of protein or another type of vector, such as a viral vector) and, optionally, with an adjuvant or in conjunction with (e.g., prior to) an alternative booster immunization (e.g., a live-vectored vaccine such as a recombinant modified vaccinia Ankara vector (MVA)) comprising an insert that may be distinct from that of the “prime” portion of the immunization or may be a related vaccine insert(s). For example, GM-CSF or other adjuvants known to those of skill in the art. The adjuvant can be a “genetic adjuvant” (i.e., a protein delivered by way of a DNA sequence).

In exemplary embodiments, method disclosed herein is an immunization method comprising (i) administering a priming composition comprising a MVA vector comprising one or more sequences encoding a HPV antigen or immunogenic fragment thereof; (ii) administering a first dose of a boosting composition comprising a HPV peptide or immunogenic fragment thereof. In a particular embodiment, the HPV peptides are the same in step (i)-(iii). Optionally, the method further comprises one or more additional steps, including, for example, the administration of one or more additional doses of the priming composition or a different priming composition (i.e., a second priming composition) and/or one or more additional doses of the boosting composition or a different boosting composition (i.e., a second boosting composition).

### EXAMPLES

### Example 1: MVA Vaccine Construction and In Vitro Evaluation for Hypoglycosylated Forms of MUC-1

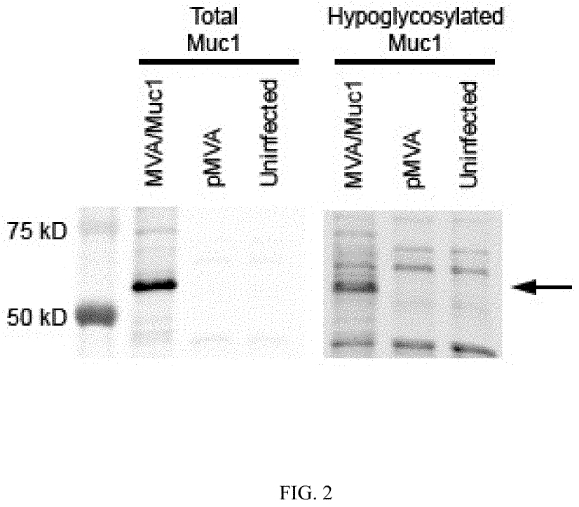

The recombinant MVA vaccine consists of an MVA vector with two antigen expression cassettes (MVA-MUC-1VP40). One expression cassette encodes a chimeric form of human MUC-1, the construction of which is described in WO 2017/120577 (hereafter this construction is called GVX-MUC-1) and which for the purposes of MVA vaccine construction has had its DNA sequence cloned into a shuttle plasmid entitled pGeo-MUC-1 (image of plasmid is seen above). One expression cassette encodes the VP40 protein of Marburgvirus. The expression of GVX-MUC-1 and VP40 is sufficient to generate secreted virus-like particles (VLPs). The GVX-MUC-1 protein is expressed as a chimeric protein consisting of the extracellular domain of human MUC-1, the transmembrane domain of Marburgvirus GP, and the intracellular domain of human MUC-1. Marburg VP40 protein is expressed in the cytoplasm of the cells where it associates with the intracellular domain and transmembrane domain of the GVX-MUC-1, causing cell-surface budding of VLPs that have GVX-MUC-1 on their surface and VP40 enclosed in their interior (luminal) space. This novel combination of vector platform and native antigen conformation yields a vaccine that is expected to elicit a strong, broad, and durable immune response. The MVA-MUC-1-VP40 vaccine candidate was constructed using shuttle vectors developed in the laboratory of Dr. Bernard Moss and are being licensed by the NIAID to GeoVax for use in vaccine development. These shuttle vectors have proven to yield stable vaccine inserts with high, but non-toxic, levels of expression in our work with HIV and hemorrhagic fever virus vaccines. The MUC-1 sequence was placed between two essential genes of MVA (I8R and G1L) and VP40 was inserted into a restructured and modified deletion III between the A50R and B1R genes, illustrated in the following schematic (FIG. 2), wherein the numbers refer to coordinates in the MVA genome.

The GVX-MUC-1 and VP40 genes were codon optimized for MVA Silent mutations have been introduced to interrupt homo-polymer sequences (>4G/C and >4A/T) to reduce RNA polymerase errors that could lead to frameshifts. Inserted sequences have been edited for vaccinia-specific terminators to remove motifs that could lead to premature termination. All vaccine inserts are placed under the modified HS early/late vaccinia promoter as described previously. Vectors were being prepared in a dedicated room under “GLP-like conditions” at GeoVax, with full traceability and complete documentation of all steps using Bovine Spongiform Encephalopathy/Transmissible Spongiform Encephalopathy (BSE/TSE)-free raw materials.

The expression of full length and native conformation of GVX-MUC-1 protein expressed in cells were assessed by western blotting using MUC-1-specific antibodies. The MVA-MUC-1VP40 vaccine was used to infect DF1 cells at a multiplicity of infection of 1.0 for 1 hour at 37° C. after which time the medium was exchanged for fresh pre-warmed medium. After 48 hours incubation at 37° C. the supernatant of the cells was harvested and clarified by centrifuging at 500×g for 10 minutes. Once the supernatant was removed from the cells, the cells themselves were harvested from the plate, washed once with cold phosphate-buffered saline (PBS) and were then lysed on ice for 15 minutes in a solution of PBS+1% Triton X-100 detergent. After this incubation, a post-nuclear supernatant was prepared by centrifuging the lysate at 1000×g for 10 minutes and harvesting the liquid layer on top, which is hereafter termed the “cell lysate”. The cell lysates were applied to 10% SDS-PAGE gels and were separated by electrophoresis, then transferred to nitrocellulose membranes, blocked with Odyssey blocking buffer, then incubated with a primary antibody that recognizes either (1) the total amount of MUC-1 present in the sample, or (2) the total amount of hypoglycosylated MUC-1 in the sample. As control, supernatant and cell lysate from DF1 cells infected with parental MVA (a vector control containing none of the antigen expression cassettes). The results of this analysis are seen in the following image of the western blot (FIG. 2):

This demonstrates that the MVA-MUC-1 VP40 vaccine infects DF1 cells and expresses MUC-1 protein and furthermore demonstrates that some proportion of the MUC-1 expressed is in hypoglycosylated form.

Evidence of the hypoglycosylated form of MUC-1 encoded by the MVA-MUC-1VP40 vaccine is seen by immunostaining cells infected with the vaccine or simultaneously staining control cells that are known to express either normally-glycosylated or hypo-glycosylated MUC-1, as described here:
Control cell lines MCF7 and MCF10A both express MUC-1. 293T cells do not.
MCF7 cell express hypo-glycosylated MUC-1, recognized by a hypoglycosylated MUC-1-specific Ab (4H5).
MCF10A expresses normal MUC-1. A pan-MUC-1 Ab (HMPV) is used to detect total MUC-1.
293T cells were infected with MVA-MUC-1 VP40 or MVA control virus (parental MVA).
All samples were stained with the indicated Abs.

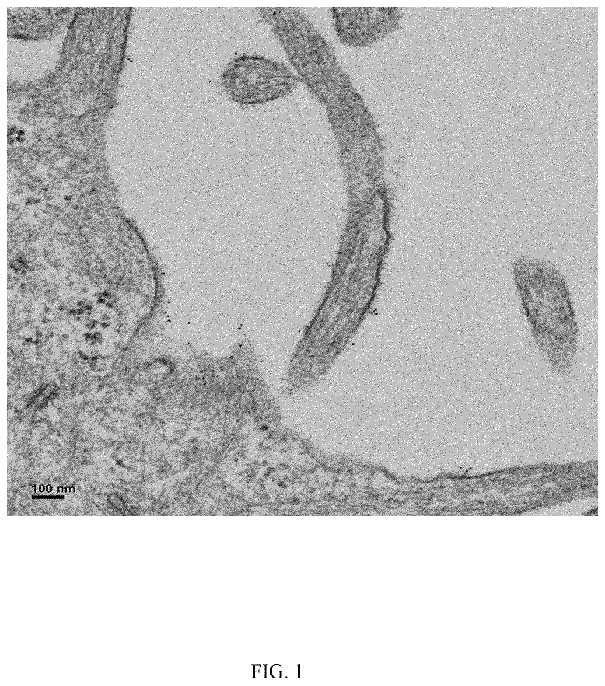

VLP formation was shown by immune-electron microscopy (EM) using of DF1 cells infected with the MVA-MUC-1-VP40 vaccine and stained with a monoclonal antibody that recognizes MUC-1 (HMPV). In the EM image below (FIG. 1) two things are clearly illustrated: (1) that the VLPs are filamentous, a phenomenon derivative of the fact that the VP40 protein is used as the matrix protein that drive VLP budding from the surface of cells; and (2) that the VLPs stain positively with the antibody directed against MUC-1, demonstrating that this protein is incorporated into the budding VLPs.

### Example 2: Assessment of Induction of Anti-Tumor MUC-1 T and B Cell Responses in Non-Tumor Bearing hMUC-1 Transgenic Mice Using MTI and/or MVA-MUC-1-VP40

TABLE 1
Experiment 1 treatment groups.
3 mice per group, 7 groups, 21 mice total.

| Group | Treatment | d0 | d7 | d14 | d21 | d28 | d35 |
|---|---|---|---|---|---|---|---|
| 1 | Control | — | — | — | — | — | Analyze |
| 2 | MTI | MTI | MTI | MTI | MTI | — | Analyze |
| 3 | MTI | MTI | — | — | MTI | — | Analyze |
| 4 | MVA | MVA | — | — | MVA | — | Analyze |
| 5 | MVA > MTI | MVA | — | — | MTI | — | Analyze |
| 6 | MTI > MVA | MTI | — | — | MVA | — | Analyze |
| 7 | MVA + MTI | MVA + MTI | — | — | MVA + MTI | — | Analyze |
| | | | | Collect | | | Collect |
| | | | | sera | | | sera |

i. Analysis

Two weeks after the last immunization (day 35), the mice are sacrificed.
Splenocytes are harvested and sera is collected.

### Data:

### Antibody ELISAs:

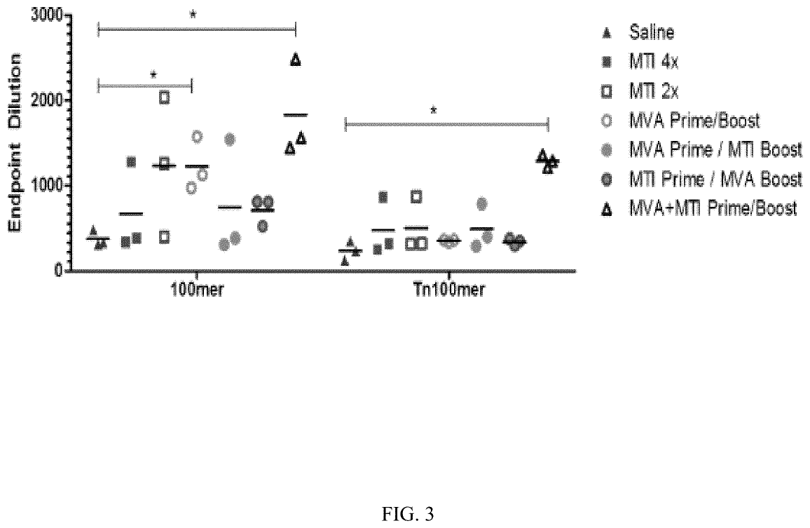

Humoral immune responses are assessed by measuring titers of MUC-1-specific antibodies using ELISA ELISA plates were coated with BSA conjugated to TSAPDT(aGalNAc)RPAP, to TSAPDTRPAP (SEQ ID NO:1), or unconjugated BSA
Results are shown in Table 1 and FIG. 3.

| | Control | | | MTI 4× | | | MTI 2× | | |
|---|---|---|---|---|---|---|---|---|---|
| Cage | 1 | 10 | 15 | 4 | 11 | 2 | 5 | 3 | 18 |
| Number | | | | | | | | | |
| Day 14 | | | | 1366 | | | 0 | | |
| MUC(Tn) | | | | | | | | | |
| Day 35 | 0 | 0 | 0 | 17889 | 2825 | 2351 | 5050 | 1738 | 23524 |
| MUC(Tn) | | | | | | | | | |
| Day 14 | | | | 1535 | | | 0 | | |
| Unglyc | | | | | | | | | |
| MUC | | | | | | | | | |
| Day 35 | 0 | 0 | 0 | 18467 | 2995 | 2147 | 5307 | 1313 | 11644 |
| Unglyc | | | | | | | | | |
| MUC | | | | | | | | | |

| | MVA | | | MVA > MTI | | | MTI > MVA | | |
|---|---|---|---|---|---|---|---|---|---|
| Cage | 12 | 14 | 8 | 9 | 13 | 7 | 16 | 21 | 17 |
| Number | | | | | | | | | |
| Day 14 | 0 | | | | | | | | |
| MUC(Tn) | | | | | | | | | |
| Day 35 | 0 | 0 | 0 | 0 | 0 | 0 | 618 | 2569 | 0 |
| MUC(Tn) | | | | | | | | | |
| Day 14 | 0 | | | | | | | | |
| Unglyc | | | | | | | | | |
| MUC | | | | | | | | | |
| Day 35 | 0 | 0 | 0 | 0 | 299 | 0 | 578 | 2445 | 0 |
| Unglyc | | | | | | | | | |
| MUC | | | | | | | | | |

| | | | | Exp 16 Mayo 2012 | | |
|---|---|---|---|---|---|---|
| | MVA + MTI | | | (4× bi-weekly) | | |
| Cage | 20 | 19 | 6 | 1b | 1d | 1f |
| Number | | | | | | |
| Day 14 | 0 | | | | | |
| MUC(Tn) | | | | | | |
| Day 35 | 3291 | 732 | 372 | 45983 | 7442 | 40928 |
| MUC(Tn) | | | | | | |
| Day 14 | 0 | | | | | |
| Unglyc | | | | | | |
| MUC | | | | | | |
| Day 35 | 3288 | 890 | 0 | 32349 | 6866 | 30270 |
| Unglyc | | | | | | |
| MUC | | | | | | |

MUC-1-specific CD8 and CD4 immune responses were assessed by intracellular staining (ICS).
Splenocytes were stimulated in vitro with TA-MUC-1 TSAPDT(GalNAc)RPAP, unglycosylated MUC-1 (TSAPDTRPAP) (SEQ ID NO:1) and non-MUC-1 peptides (HIV-1 Env peptides, negative control) and a MUC-1 peptide library prior to ICS.

| Vaccination | | IFNg+ Animals | | TNF+ Animals | |
|---|---|---|---|---|---|
| Condition | MUC-1 Peptide | CD4+ | CD8+ | CD4+ | CD8+ |
| Saline | Short, non-g | 0 | 0 | 0 | 0 |
| | Short, g | 0 | 0 | 0 | 0 |
| | Long, g | 0 | 0 | 0 | 0 |
| | MUC-1 Library | 0 | 0 | 0 | 0 |
| MTI (4 dose) | Short, non-g | 1 | 1 | 3 | 0 |
| | Short, g | 0 | 0 | 0 | 0 |
| | Long, g | 1 | 1 | 0 | 0 |
| | MUC-1 Library | 0 | 0 | 0 | 0 |
| MTI (2 dose) | Short, non-g | 0 | 0 | 1 | 0 |
| | Short, g | 0 | 0 | 0 | 0 |
| | Long, g | 0 | 0 | 0 | 0 |
| | MUC-1 Library | 0 | 0 | 0 | 0 |
| MVA | Short, non-g | 1 | 1 | 0 | 0 |
| | Short, g | 1 | 1 | 0 | 0 |
| | Long, g | 1 | 1 | 0 | 0 |
| | MUC-1 Library | 0 | 0 | 0 | 0 |
| MVA > MTI | Short, non-g | 1 | 1 | 0 | 0 |
| | Short, g | 1 | 1 | 1 | 1 |
| | Long, g | 0 | 0 | 0 | 0 |
| | MUC-1 Library | 0 | 0 | 0 | 0 |
| MTI > MVA | Short, non-g | 0 | 0 | 0 | 0 |
| | Short, g | 0 | 0 | 0 | 0 |
| | Long, g | 1 | 1 | 0 | 0 |
| | MUC-1 Library | 0 | 0 | 0 | 0 |
| MVA + MTI | Short, non-g | 0 | 0 | 1 | 0 |
| | Short, g | 0 | 1 | 0 | 0 |
| | Long, g | 0 | 0 | 0 | 0 |
| | MUC-1 Library | 0 | 0 | 0 | 0 |

MUC-1 Peptide Name Key:

| | |
|---|---|
| Name | Description |
| Short, non-g | Short MUC-1 peptide, non-glycosylated |
| Short, g | Short MUC-1 peptide, glycosylated |
| Long, g | Long MUC-1 peptide, glycosylated |
| MUC-1 Library | Full MUC-1 sequence peptide library |

### Example 3: Assessment and Optimization of a Combined MUC-1 Vaccine and Immune Checkpoint Inhibitor Therapy to Effect Tumor Regression in Mice with Established MUC-1+ Tumors Using the Therapeutic hMUC-1Tg Mouse Tumor Model

Compositions of MTI, MVA and MTI+MVA were evaluated for ability to enhance anti-tumor activity of anti-PD-1 antibody. MC38 MUC-1 cells (implanted SC) will be used for the experiment.

| Treatment | | |
|---|---|---|
| Group | MUC- 1 | Anti-mPD-1 |
| 1 | | |
| 2 | | yes |
| 3 | MTI (4 dose) | yes |
| 4 | MVA (2 dose) | yes |
| 5 | MTI (2 dose) + | yes |
| | MVA (2 dose) | |

hMUC-1 Tg mice

hMUC-1 MC38 tumor cells

Anti-mPD-1 dosed 2× per week for 5 wks, starting on d8.

5 mice per group

Tumor calculated from caliper measurements.

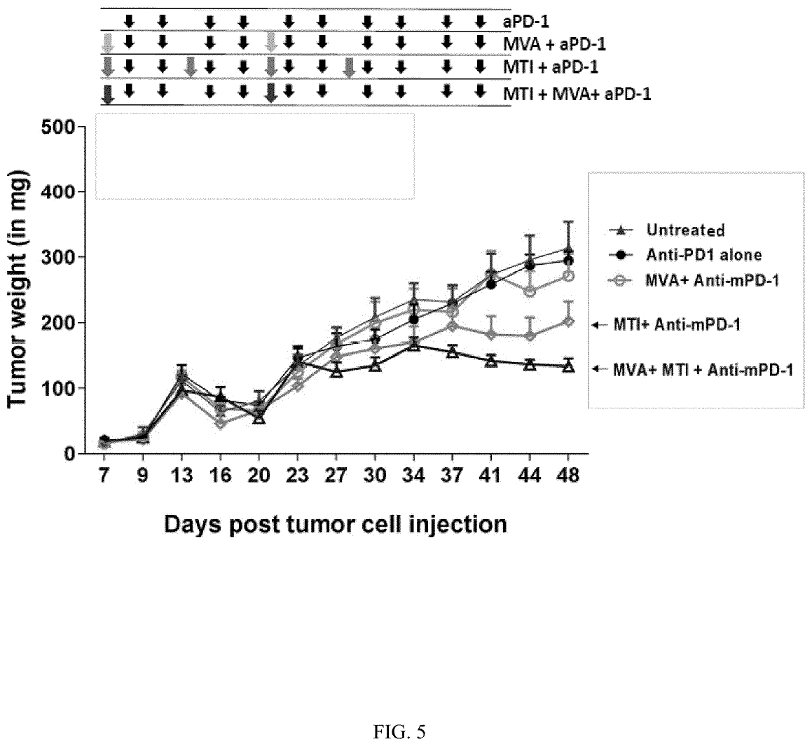

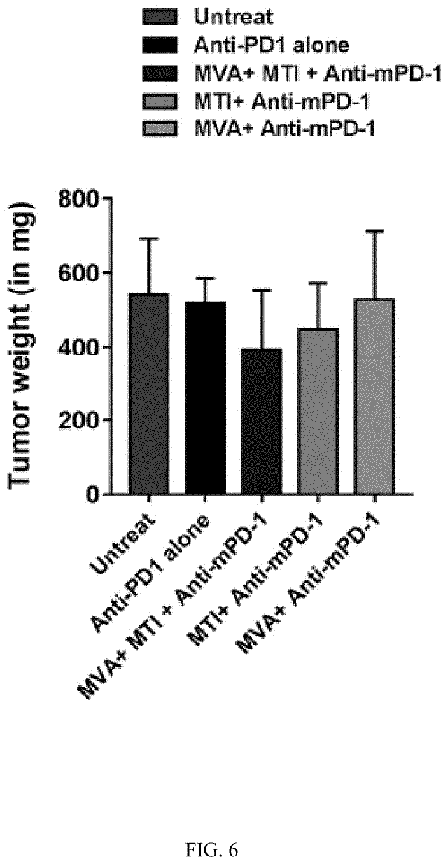

Results are shown in FIGS. 5 and 6.

### Example 4: Assessment of Induction of Anti-Tumor MUC-1 T and B Cell Responses in Non-Tumor Bearing hMUC-1 Transgenic Mice Using Tn-100-Mer (Tn-MUC-1) and/or MVA-MUC-1 VP40

For the MVA-VLP-MUC-1 vaccine, the cell surface protein recombined into the MVA genome is the gene for human MUC-1, a highly glycosylated type-I transmembrane protein expressed on the apical membrane of epithelial cells. Human MUC-1 associated with transformed cells is expressed in a hypo-glycosylated form, acting as a cancer neo-antigen that results from aberrant post-translation modification of the protein. The MUC-1 incorporated into the MVA-VLP-MUC-1 vaccine was modified in such a way that the MUC-1 transmembrane domain (TM) was swapped out with the TM of MARV GP protein. The MUC-1 gene was placed behind the modified HS promoter of Vaccinia, facilitating a moderate-to-high level of expression in infected cells.

In addition to MUC-1, the MVA-VLP-MUC-1 cancer vaccine expresses the VP40 gene from Marburg virus. Expressed VP40 associates with (i) the inner leaflet of the plasma membrane, (ii) the TM of MARV GP, and (iii) itself in a polymeric form, all of which facilitate the budding of VLPs from the surface of the infected cell. Because the MUC-1 incorporated into the MVA-VLP-MUC-1 vaccine contains the TM of MARV GP, it is MUC-1 that directly associates with VP40, facilitating the production of VLPs that bear MUC-1 on their surface.

Infection of cells with MVA-VLP-MUC-1 drives expression of both MUC-1 and VP40 from the cells. Importantly, by using antibodies (Abs) that are specific for hypo-glycosylated MUC-1, consistent reactivity of this Ab occurs with the MUC-1 expressed by infected cells. This was shown both by staining of infected cells as well as by western blot.

Parameters for the study of MVA-VLP vectors in mice have been previously established. Mice are to be administered a dose of 10⁷ TCID₅₀ MVA-VLP-MUC-1 by intramuscular (IM) administration in hind legs. The vaccine is typically formulated at a concentration of 10⁸ TCID₅₀/mL, so I00 μL is administered per animal.

### Vaccine Immunogenicity Experiment

The immunogenicity of MVA-VLP-MUC-1 is assessed in a mouse experimental system.

MUC-1 Tg mice are immunized with 10⁷ TCID₅₀ by IM injection using a prime-boost regimen. MVA-VLP-MUC-1 group 1 are compared with Tn-100-mer peptide loaded on BM-derived DC matured with Poly-ICLC (group 2). Groups 3 and 4 also receive soluble Tn-100-mer peptide to enhance antibody production and focus the response to the tandem repeat region. The test groups are as follows:

Immunogenicity Study Groups (10 mice per group)

| Group | Vaccination Condition |
|---|---|
| 1 | MVA-VLP-MUC-1 |
| 2 | Tn-100-mer on DC |
| 3 | Tn-100-mer on DC + soluble Tn-100-mer |
| 4 | MVA-VLP-MUC-1 + soluble Tn-100-mer |
| 5 | MVA-VLP-MUC-1 prime, Tn-100-mer plus Hiltonol boost |

Administration occurs on days 0 and boost on day 28 of the study. 10 days after the final vaccination, 3 mice from each group will be sacrificed, splenectomized, and their spleens used to measure T cell responses to vaccination. Serum is collected prior to administration, prior to the boost and 2 weeks after the boost and anti-MUC-1 IgG titers determined in ELISA on 100-mer and Tn-100-mer. 2 weeks after the boost, the remaining mice are challenged with MUC-1+ tumors SQ.

### Example 5: MVA-HVP and Vaccinia-HPV Therapeutic Vaccines

Timelines and divisions of tasks for the MVA-HVP and Vaccinia-HPV therapeutic vaccines;

### Experiments: MVA-VLP-HPV-Expressing Vaccine Candidates are Used for Immunization of Mice

In the first experiment, described below, the MVA-16-E2 is tested. Other vaccine candidates (e.g., MVA-16-E6/E7, MVA-16-E2+E6/E7 or vaccinia versions) are also tested using similar protocols.

### Experiment 1 Immunogenicity of the MVA-E2 and VV Vaccine in Normal Mice

### Overview

### I. Overview

Three MVA/HPV vaccines and three VV/HPV targeting HPV-16 E2, HPV-16 E6/E7 or combination of both (HPV-16 E2+E6/E7) are tested in 10-12-week-old C57BL/6 female mice using a Prime-Boost regimen consisting of vaccinations on day 0 (d0) and d28 with the final day of the study on d56. In the first experiment (Study 1), the immunogenicity of the MVA-HPV-16 E2 and VV/HPV-16-E2 vaccines will be tested as a proof of concept study followed by future experiments on the next two constructs addressing T-cell responses to HPV-16 E6/E7 and Ab and T-cell responses to the third construct HPV-16 E2+E6/E7.

As a negative control vaccine, the MVA-VLP-MARV or VV vaccine will be used. Immunogenicity to the backbone MVA will be evaluated in the vaccine and negative control sera after prime and boost vaccination. Serum samples will be collected for measuring the humoral immune response following the schedule outlined below. These samples are evaluated for the presence and abundance of antigen-specific antibodies (Abs) by ELISA

### Study 1

Mouse strain: 10-12-Week-old female C57BL/6
Vaccination administration route: intramuscular
Vaccination dose: 10⁷ TCID₅₀ for MVA-HPV-16 and 10⁵ TCID₅₀ for VV-HPV-16
Bleeding route: retro-orbital or tail vein
Vaccine regimen: Prime-Boost

### Biosafety Level: BSL2

TABLE 2
Animal Groups Homologous Prime-Boost

| Group ID | Vaccine | Regimen | Prime | Boost | n |
|---|---|---|---|---|---|
| 1 | MVA-HPV-16 E2 | Prime-Boost | Day 0 | Day 28 | 8 |
| 2 | MVA-VLP-MARV | Prime-Boost | Day 0 | Day 28 | 8 |
| TOTAL | | | | | 16 |

Homologous Prime-Boost

| Group ID | Vaccine | Regimen | Prime | Boost | n |
|---|---|---|---|---|---|
| 1 | VV-HPV-16 E2 | Prime-Boost | Day 0 | Day 28 | 8 |
| 2 | VV | Prime-Boost | Day 0 | Day 28 | 8 |
| TOTAL | | | | | 16 |

Heterologous Prime-Boost

| Group ID | Prime Vaccine Day 0 | Boost Vaccine Day 28 | n |
|---|---|---|---|
| 1 | MVA-HPV-16 E2 | VV-HPV-16 E2 | 8 |
| 2 | VV-HPV-16 E2 | MVA-HPV-16 E2 | 8 |
| TOTAL | | | 16 |

### II. Schedule

Table 2. Prime/Boost vaccination schedule. C57B1/6 mice will be vaccinated on d0 and d28 and sacrificed on the final study day. Serum samples obtained by bleeding will be collected on days 0, 14, 28, 42, and 56) and tested (Table 3).

TABLE 3
Sampling and Assays

| Study Day | Sample | Assay |
|---|---|---|
| 0 | Serum | ELISA |
| 14 | Serum | ELISA |
| 28 | Serum | ELISA |
| 42 | Serum | ELISA |
| 56 | Serum (exsanguination) | ELISA |

### Example 6: Muc1/4R Gene Optimization

The following example provides a stepwise methodology for creating an optimized Muc1/4TR gene. Start with the natural sequence for Homo sapiens mucin 1 NCBI Reference Sequence: NM_001204285.1 (http://www.ncbi.nlm.nih.gov/nuccore/NM_0012024285.1)

Muc1/1TR protein (475 aa) (SEQ ID NO: 6):
MTPGTQSPFFLLLLLTVLTVVTGSGHASSTPGGEKETSATQRSSVPSSTEKNAVSMTS
SVLSSHSPGSGSSTTQGQDVTLAPATEPASGSAATWGQDVTSVPVTRPALGSTTPPAH
DVTSAPDNKPAPGSTAPPAHGVTSAPDTRPAPGSTAPPAHGVTSAPDNRPALGSTAPP
VHNVTSASGSASGSASTLVHNGTSARATTTPASKSTPFSIPSHHSDTPTTLASHSTKTDA
SSTHHSTVPPLTSSNHSTSPQLSTGVSFFFLSFHISNLQFNSSLEDPSTDYYQELQRDISE
MFLQIYKQGGFLGLSNIKFRPGSVVVQLTLAFREGTINVHDVETQFNQYKTEAASRYN

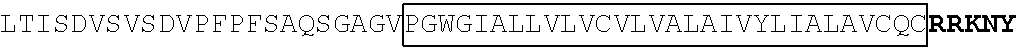

GQLDIFPARDTYHPMSEYPTYHTHGRYVPPSSTDRSPYEKVSAGNGGSSLSYTNPA
VAATSANL
In bold at the beginning: Signal Peptide
In underlined: Tandem Repeats
In the box: Transmembrane Domain
In bold at the end: Cytoplasmic Tail

The Muc1 gene that contained 4 Tandem Repeats was selected.

GeoVax Muc1/4TR sequence (1608 nt) (SEQ ID NO: 10):
ATGACACCGGGCACCCAGTCTCCTTTCTTCCTGCTGCTGCTCCTCACAGTGCTTACA
GTTGTTACGGGTTCTGGTCATGCAAGCTCTACCCCAGGTGGAGAAAAGGAGACTTC
GGCTACCCAGAGAAGTTCAGTGCCCAGCTCTACTGAGAAGAATGCTGTGAGTATG
ACCAGCAGCGTACTCTCCAGCCACAGCCCCGGTTCAGGCTCCTCCACCACTCAGGG
ACAGGATGTCACTCTGGCCCCGGCCACGGAACCAGCTTCAGGTTCAGCTGCCACCT
GGGGACAGGATGTCACCTCGGTCCCAGTCACCAGGCCAGCCCTGGGCTCCACCAC
CCCGCCAGCCCACGATGTCACCTCAGCCCCGGACAACAAGCCAGCCCCGGGCTCC

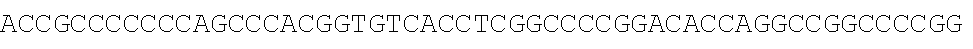

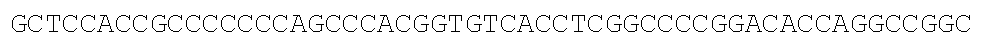

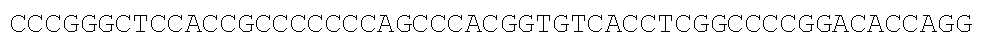

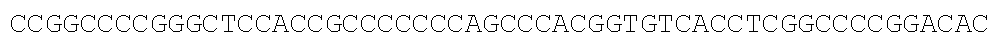

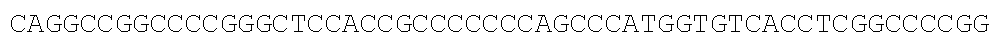

ACAACAGGCCCGCCTTGGGCTCCACCGCCCCTCCAGTCCACAATGTCACCTCGGCC
TCAGGCTCTGCATCAGGCTCAGCTTCTACTCTGGTGCACAACGGCACCTCTGCCAG
GGCTACCACAACCCCAGCCAGCAAGAGCACTCCATTCTCAATTCCCAGCCACCACT
CTGATACTCCTACCACCCTTGCCAGCCATAGCACCAAGACTGATGCCAGTAGCACT
CACCATAGCACGGTACCTCCTCTCACCTCCTCCAATCACAGCACTTCTCCCCAGTT
GTCTACTGGGGTCTCTTTCTTTTTCCTGTCTTTTCACATTTCAAACCTCCAGTTTAAT
TCCTCTCTGGAAGATCCCAGCACCGACTACTACCAAGAGCTGCAGAGAGACATTTC
TGAAATGTTTTTGCAGATTTATAAACAAGGGGGTTTTCTGGGCCTCTCCAATATTA
AGTTCAGGCCAGGATCTGTGGTGGTACAATTGACTCTGGCCTTCCGAGAAGGTACC
ATCAATGTCCACGACGTGGAGACACAGTTCAATCAGTATAAAACGGAAGCAGCCT
CTCGATATAACCTGACGATCTCAGACGTCAGCGTGAGTGATGTGCCATTTCCTTTC

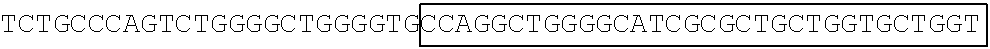

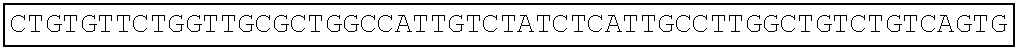

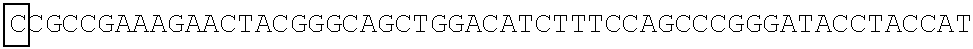

CCTATGAGCGAGTACCCCACCTACCACACCCATGGGCGCTATGTGCCCCCTAGCAG
TACCGATCGTAGCCCCTATGAGAAGGTTTCTGCAGGTAATGGTGGCAGCAGCCTCT
CTTACACAAACCCAGCAGTGGCAGCCACTTCTGCCAACTTGTAG
Muc1/4TR protein (535aa) (SEQ ID NO: 11):
MTPGTQSPFFLLLLLTVLTVVTGSGHASSTPGGEKETSATQRSSVPSSTEKNAVSMTSS
VLSSHSPGSGSSTTQGQDVTLAPATEPASGSAATWGQDVTSVPVTRPALGSTTPPAHD

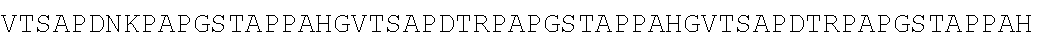

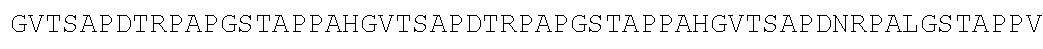

HNVTSASGSASGSASTLVHNGTSARATTTPASKSTPFSIPSHHSDTPTTLASHSTKTDAS
STHEISTVPPLTSSNHSTSPQLSTGVSFFFLSFHISNLQFNSSLEDPSTDYYQELQRDISEM
FLQIYKQGGFLGLSNIKFRPGSVVVQLTLAFREGTINVHDVETQFNQYKTEAASRYNLT
ISDVSVSDVPFPFSAQSGAGVPGWGIALLVLVCVLVALAIVYLIALAVCQCRRKNYG
QLDIFPARDTYHPMSEYPTYHTHGRYVPPSSTDRSPYEKVSAGNGGSSLSYTNPAVAA
TSANL
Grey shading denotes tandem repeats.

The sequence 4 tandem Repeat sequence can then be aligned with the prior sequence. To increase the efficiency of the incorporation of Mud into Marburg VP40-based VLPs, the transmembrane domain of Mud was replaced with the transmembrane domain of the Marburg virus glycoprotein.

Geo Vax Muc1/4TR sequence (Transmembrane domain sequence in red:
position 1129-1218 on Muc1/1 TR, Grey shading denotes tandem repeats.)
(SEQ ID NO: 10):
ATGACACCGGGCACCCAGTCTCCTTTCTTCCTGCTGCTGCTCCTCACAGTGCTTACAG
TTGTTACGGGTTCTGGTCATGCAAGCTCTACCCCAGGTGGAGAAAAGGAGACTTCGG
CTACCCAGAGAAGTTCAGTGCCCAGCTCTACTGAGAAGAATGCTGTGAGTATGACC
AGCAGCGTACTCTCCAGCCACAGCCCCGGTTCAGGCTCCTCCACCACTCAGGGACAG
GATGTCACTCTGGCCCCGGCCACGGAACCAGCTTCAGGTTCAGCTGCCACCTGGGGA
CAGGATGTCACCTCGGTCCCAGTCACCAGGCCAGCCCTGGGCTCCACCACCCCGCCA
GCCCACGATGTCACCTCAGCCCCGGACAACAAGCCAGCCCCGGGCTCCACCGCCCC

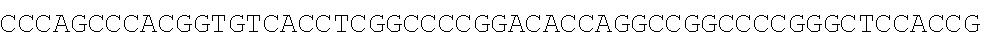

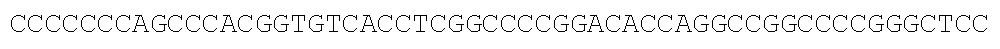

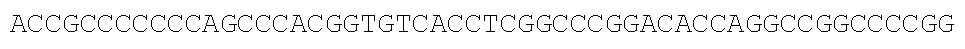

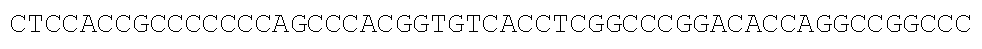

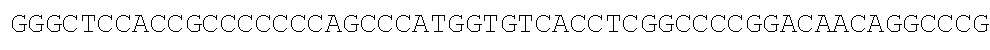

CCTTGGGCTCCACCGCCCCTCCAGTCCACAATGTCACCTCGGCCTCAGGCTCTGCAT
CAGGCTCAGCTTCTACTCTGGTGCACAACGGCACCTCTGCCAGGGCTACCACAACCC
CAGCCAGCAAGAGCACTCCATTCTCAATTCCCAGCCACCACTCTGATACTCCTACCA
CCCTTGCCAGCCATAGCACCAAGACTGATGCCAGTAGCACTCACCATAGCACGGTA
CCTCCTCTCACCTCCTCCAATCACAGCACTTCTCCCCAGTTGTCTACTGGGGTCTCTT
TCTTTTTCCTGTCTTTTCACATTTCAAACCTCCAGTTTAATTCCTCTCTGGAAGATCCC
AGCACCGACTACTACCAAGAGCTGCAGAGAGACATTTCTGAAATGTTTTTGCAGATT
TATAAACAAGGGGGTTTTCTGGGCCTCTCCAATATTAAGTTCAGGCCAGGATCTGTG
GTGGTACAATTGACTCTGGCCTTCCGAGAAGGTACCATCAATGTCCACGACGTGGAG
ACACAGTTCAATCAGTATAAAACGGAAGCAGCCTCTCGATATAACCTGACGATCTCA
GACGTCAGCGTGAGTGATGTGCCATTTCCTTTCTCTGCCCAGTCTGGGGCTGGGGTG

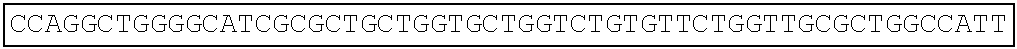

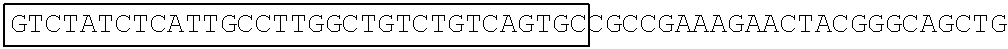

GACATCTTTCCAGCCCGGGATACCTACCATCCTATGAGCGAGTACCCCACCTACCAC
ACCCATGGGCGCTATGTGCCCCCTAGCAGTACCGATCGTAGCCCCTATGAGAAGGTT
TCTGCAGGTAATGGTGGCAGCAGCCTCTCTTACACAAACCCAGCAGTGGCAGCCACT
TCTGCCAACTTGTAG
(Transmembrane domain sequence in red: position 157-186 on Muc1/lTR,
Grey shading denotes tandem repeats.) (SEQ ID NO: 11):
MTPGTQSPFFLLLLLTVLTVVTGSGHASSTPGGEKETSATQRSSVPSSTEKNAVSMTSSV
LSSHSPGSGSSTTQGQDVTLAPATEPASGSAATWGQDVTSVPVTRPALGSTTPPAHDVTS

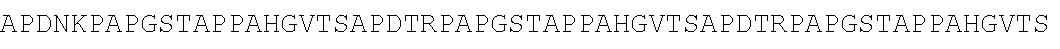

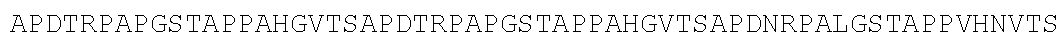

ASGSASGSASTLVHNGTSARATTTPASKSTPFSIPSHHSDTPTTLASHSTKTDASSTHHST
VPPLTSSNHSTSPQLSTGVSFFFLSFHISNLQFNSSLEDPSTDYYQELQRDISEMFLQIYKQ
GGFLGLSNIKFRPGSVVVQLTLAFREGTINVHDVETQFNQYKTEAASRYNLTISDVSVSD

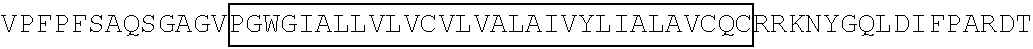

YHPMSEYPTYHTHGRYVPPSSTDRSPYEKVSAGNGGSSLSYTNPAVAATSANL
Replace the transmembrane sequence with the transmembrane sequence of
Marburg GP (SEQ ID NO: 12): WWTSDWGVLTNLGILLLLSIAVLIALSCIC;
(SEQ ID NO: 13):
ATGACACCGGGCACCCAGTCTCCTTTCTTCCTGCTGCTGCTCCTCACAGTGCTTACAG
TTGTTACGGGTTCTGGTCATGCAAGCTCTACCCCAGGTGGAGAAAAGGAGACTTCGG
CTACCCAGAGAAGTTCAGTGCCCAGCTCTACTGAGAAGAATGCTGTGAGTATGACC
AGCAGCGTACTCTCCAGCCACAGCCCCGGTTCAGGCTCCTCCACCACTCAGGGACAG
GATGTCACTCTGGCCCCGGCCACGGAACCAGCTTCAGGTTCAGCTGCCACCTGGGGA
CAGGATGTCACCTCGGTCCCAGTCACCAGGCCAGCCCTGGGCTCCACCACCCCGCCA
GCCCACGATGTCACCTCAGCCCCGGACAACAAGCCAGCCCCGGGCTCCACCGCCCC

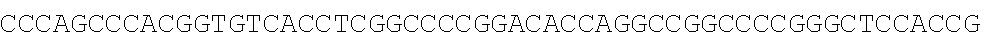

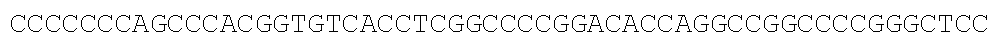

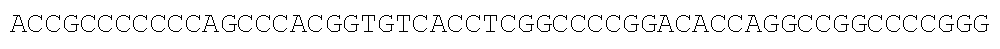

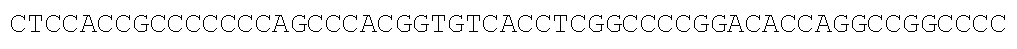

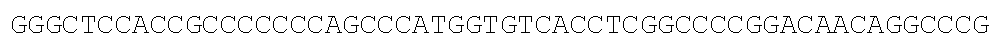

CCTTGGGCTCCACCGCCCCTCCAGTCCACAATGTCACCTCGGCCTCAGGCTCTGCAT
CAGGCTCAGCTTCTACTCTGGTGCACAACGGCACCTCTGCCAGGGCTACCACAACCC
CAGCCAGCAAGAGCACTCCATTCTCAATTCCCAGCCACCACTCTGATACTCCTACCA
CCCTTGCCAGCCATAGCACCAAGACTGATGCCAGTAGCACTCACCATAGCACGGTA
CCTCCTCTCACCTCCTCCAATCACAGCACTTCTCCCCAGTTGTCTACTGGGGTCTCTT
TCTTTTTCCTGTCTTTTCACATTTCAAACCTCCAGTTTAATTCCTCTCTGGAAGATCCC
AGCACCGACTACTACCAAGAGCTGCAGAGAGACATTTCTGAAATGTTTTTGCAGATT
TATAAACAAGGGGGTTTTCTGGGCCTCTCCAATATTAAGTTCAGGCCAGGATCTGTG
GTGGTACAATTGACTCTGGCCTTCCGAGAAGGTACCATCAATGTCCACGACGTGGAG
ACACAGTTCAATCAGTATAAAACGGAAGCAGCCTCTCGATATAACCTGACGATCTCA

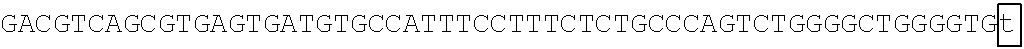

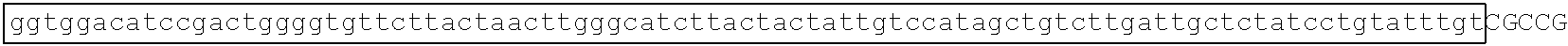

AAAGAACTACGGGCAGCTGGACATCTTTCCAGCCCGGGATACCTACCATCCTATGA
GCGAGTACCCCACCTACCACACCCATGGGCGCTATGTGCCCCCTAGCAGTACCGATC
GTAGCCCCTATGAGAAGGTTTCTGCAGGTAATGGTGGCAGCAGCCTCTCTTACACAA
ACCCAGCAGTGGCAGCCACTTCTGCCAACTTGTAG
Corresponding protein sequence (SEQ ID NO: 14):
MTPGTQSPFFLLLLLTVLTVVTGSGHASSTPGGEKETSATQRSSVPSSTEKNAVSMTSSV

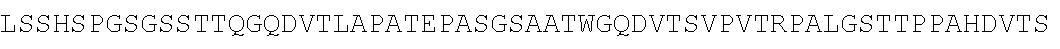

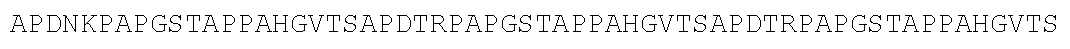

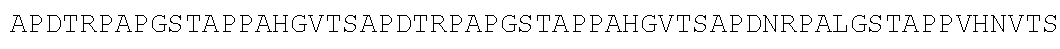

ASGSASGSASTLVHNGTSARATTTPASKSTPFSIPSHHSDTPTTLASHSTKTDASSTHHST
VPPLTSSNHSTSPQLSTGVSFFFLSFHISNLQFNSSLEDPSTDYYQELQRDISEMFLQIYKQ
GGFLGLSNIKFRPGSVVVQLTLAFREGTINVHDVETQFNQYKTEAASRYNLTISDVSVSD

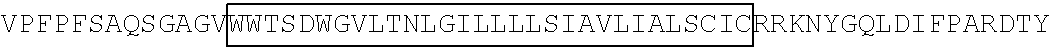

HPMSEYPTYHTHGRYVPPSSTDRSPYEKVSAGNGGSSLSYTNPAVAATSANL

The DNA sequence can then be codon optimized for vaccinia virus. The GeneArt Gene Synthesis tool in LifeTechnolgy website (http://www.entelechon.com/2008/10/backtranslation-tool/) can be used to optimize the sequence for vaccinia virus.

Next, homopolymer sequences (G/C or T/A rich areas) can be interrupted by silent mutations. The sequence was searched for ≥4 G/C areas and none were found. The sequence was searched for ≥5 A/T areas and seven A/T rich areas were found. All were then interrupted by single silent mutation. Table 4 summarizes all the mutations made on Muc1.

TABLE 4

| Sequence | | Mutation |
|---|---|---|
| nucleotides | Silent Mutation (red) | position |
| (amino acid) | nucleotides (amino acid) | on GP |
| TTT | TTC | 27 |
| TTT | TTC | 30 |
| GAA | GA | 102 |
| AAA | AA | 105 |
| GAA | GA | 150 |
| AAA | AA | 153 |
| AAA | AA | 375 |
| AAA | AA | 804 |
| TTT | TTC | 816 |
| AAA | AA | 873 |
| TTT | TTC | 966 |
| TTT | TTC | 969 |
| TTT | TTC | 972 |
| TCT | TCC | 978 |
| TTT | TTC | 1002 |
| AAA | AAG | 1215 |
| TTT | TTC | 1284 |
| AAA | AAG | 1407 |
| TTT | TTC | 1431 |
| GAA | GAG | 1530 |
| AAA | AAG | 1633 |

Then the GP sequence could be searched for vaccinia virus transcription terminator sequences, and no T₅NT motif was found. Next, a second stop codon was added. The sequences of the Tandem Repeats were modified by silent mutation (when possible), to reduce recombination and to increase the insert stability.

### First Tandem Repeat

(SEQ ID NO: 15):
GCT CAT GGT GTT ACT TCA GCG CCT GAT ACA
AGA CCT GCA CCT GGA TCT ACA GCT CCT CCT
(SEQ ID NO: 2):
A H G V T S A P D T R P A P G S T A P P

### Second Tandem Repeat

(SEQ ID NO: 16):
GCA CAT GGT GTA ACA TCT GCT CCA GAT ACA
AGA CCA GCT CCA GGT TCA ACA GCA CCT CCA

### Third Tandem Repeat

(SEQ ID NO: 17):
GCG CAT GGT GTT ACT AGT GCT CCA GAT ACA
AGA CCT GCG CCT GGA AGT ACT GCA CCA CCA

### Fourth Tandem Repeat

(SEQ ID NO: 18):
GCA CAT GGT GTA ACT AGT GCG CCT GAT ACA
AGA CCA GCG CCA GGA TCA ACT GCT CCT CCT
(SEQ ID NO: 19):
GCT CAT GGT GTT ACT TCA GCG CCT GAT ACA
AGA CCc GCA CCc GGA TCT ACc GCT CCg CCT
(SEQ ID NO: 20):
GCA CAc GGc GTc ACA TCT GCT CCc GAc ACt
cgt CCA GCT CCt GGT agc ACA GCA CCT CCA
(SEQ ID NO: 21):
GCG CAT GGa GTa ACc AGT GCa CCA GAT ACc
cga CCt GCG CCg GGc AGT ACT GCc CCA CCg
(SEQ ID NO: 22):
GCc CAc GGg GTg ACg AGc GCc CCg GAc ACg
cgc CCA GCt CCA GGg TCA ACg GCg CCc CCT
(SEQ ID NO: 2):
A H G V T S A P D T R P A P G S T A P P

The next step was to add restriction sites for cloning of the Mud into MVA-shuttles plasmids pLW-73. Sma I and Sal I restriction sites were added at 3′ and 5′ of Muc I gene respectively. 5 nucleotides upstream Sma1 and 5 nucleotides downstream Sal1 were added to facilitate the digestion and cloning: gcgct.

Final sequence (SeqBuilder file) for
Genscript: GVX-Muc4TRMTM
(SEQ ID NO: 23):
gcgctcccgggATGACACCTGGAACACAATCTCCA
TTcTTcCTACTACTACTATTGACAGTACTAACAGT
AGTAACAGGATCTGGACATGCGTCTAGTACACCAG
GTGGAGAgAAgGAAACATCTGCGACTCAAAGATCT
TCTGTACCATCTTCTACAGAgAAgAATGCGGTATC
TATGACATCTAGTGTACTATCTTCTCATTCTCCTG
GATCTGGATCTTCTACTACACAAGGACAAGATGTA
ACACTAGCGCCAGCTACAGAACCAGCTTCTGGATC
TGCTGCTACTTGGGGTCAAGATGTTACTTCTGTTC
CAGTAACAAGACCAGCGCTAGGATCTACAACACCA
CCAGCGCATGATGTAACAAGTGCGCCAGATAATAA
gCCAGCGCCTGGTTCTACTGCTCCACCAGCTCATG
GTGTTACTTCAGCGCCTGATACAAGACCTGCACCT
GGATCTACAGCTCCTCCTGCACATGGTGTAACATC
TGCTCCAGATACAAGACCAGCTCCAGGTTCAACAG
CACCTCCAGCGCATGGTGTTACTAGTGCTCCAGAT
ACAAGACCTGCGCCTGGAAGTACTGCACCACCAGC
ACATGGTGTAACTAGTGCGCCTGATACAAGACCAG
CGCCAGGATCAACTGCTCCTCCTGCTCATGGTGTT
ACAAGTGCACCTGATAATAGACCTGCGTTGGGATC
TACTGCGCCTCCAGTTCATAATGTAACATCAGCGT
CTGGAAGTGCGTCTGGTTCTGCGTCTACATTGGTT
CATAATGGTACATCTGCGAGAGCGACAACAACTCC
AGCGTCTAAgTCTACACCATTcTCTATTCCATCTC
ATCATTCTGATACACCAACAACATTGGCGAGTCAT
TCTACAAAgACAGATGCGAGTTCTACACATCATTC
TACTGTACCACCACTAACATCTTCTAATCATAGTA
CATCTCCACAACTATCTACTGGTGTATCTTTcTTc
TTcCTATCcTTTCATATTTCTAATCTACAGTTcAA
TTCTAGTTTGGAAGATCCATCTACAGATTATTATC
AAGAACTACAAAGAGATATTTCTGAAATGTTTCTA
CAAATATATAAACAAGGAGGATTTCTAGGACTATC
TAATATTAAGTTTAGACCAGGATCTGTAGTAGTTC
AACTAACTCTAGCGTTTAGAGAAGGTACTATTAAT
GTACATGATGTTGAAACACAGTTTAATCAATATAA
gACAGAAGCGGCGTCTAGATATAATCTAACAATTT
CTGATGTATCTGTATCTGATGTTCCATTTCCATTc
TCTGCGCAATCTGGTGCTGGTGTATGGTGGACATC
TGATTGGGGAGTACTAACTAATCTAGGAATTCTAC
TATTGCTATCTATTGCGGTACTAATTGCGCTATCT
TGTATATGTAGAAGAAAgAATTATGGACAACTAGA
TATTTTcCCAGCGAGAGATACTTATCATCCAATGT
CTGAATATCCAACATATCATACACATGGAAGATAT
GTACCACCTTCTTCAACAGATAGATCTCCATATGA
gAAgGTATCTGCGGGAAATGGTGGTTCTTCTCTAT
CTTATACAAATCCAGCGGTAGCGGCGACTTCTGCG
AATCTATAATAAgtcgacgcgct
Corresponding protein sequence
(SEQ ID NO: 14):
MTPGTQSPFFLLLLLTVLTVVTGSGHASSTPGGEK
ETSATQRSSVPSSTEKNAVSMTSSVLSSHSPGSGS
STTQGQDVTLAPATEPASGSAATWGQDVTSVPVTR
PALGSTTPPAHDVTSAPDNKPAPGSTAPPAHGVTS
APDTRPAPGSTAPPAHGVTSAPDTRPAPGSTAPPA
HGVTSAPDTRPAPGSTAPPAHGVTSAPDTRPAPGS
TAPPAHGVTSAPDNRPALGSTAPPVHNVTSASGSA
SGSASTLVHNGTSARATTTPASKSTPFSIPSHHSD
TPTTLASHSTKTDASSTHHSTVPPLTSSNHSTSPQ
LSTGVSFFFLSFHISNLQFNSSLEDPSTDYYQELQ
RDISEMFLQIYKQGGFLGLSNIKFRPGSVVVQLTL
AFREGTINVHDVETQFNQYKTEAASRYNLTISDVS
VSDVPFPFSAQSGAGVWWTSDWGVLTNLGILLLLS
IAVLIALSCICRRKNYGQLDIFPARDTYHPMSEYP
TYHTHGRYVPPSSTDRSPYEKVSAGNGGSSLSYTN
PAVAATSANL.

The foregoing discussion discloses and describes merely exemplary embodiments of the compositions and methods disclosed herein. One skilled in the art will readily recognize from such discussion, and from the accompanying drawings and claims, that various changes, modifications and variations can be made therein without departing from the spirit and scope of the disclosure defined in the following claims.

All references cited herein are incorporated by reference in their entirety.

We claim:

1. A method of treating a human having a cancer comprising:
i) administering to the human a therapeutically effective amount of a recombinant modified vaccinia Ankara (MVA) viral vector, comprising (a) a first nucleic acid sequence encoding a tumor associated antigen (TAA), wherein the first nucleic acid sequence is under the control of a promoter compatible with poxvirus expression systems; and
ii) administering to the human a therapeutically effective amount of an oncolytic vaccinia virus.

2. The method of claim 1, wherein the human is further administered a therapeutically effective amount of an isolated tumor associated antigen.

3. A method of treating a human having a cancer, comprising:
i) administering to the human a therapeutically effective amount of a recombinant modified vaccinia Ankara (MVA) viral vector, comprising (a) a first nucleic acid sequence encoding a tumor associated antigen (TAA), wherein the first nucleic acid sequence is under the control of a promoter compatible with poxvirus expression systems;
ii) administering to the human a therapeutically effective amount of an oncolytic vaccinia virus; and
iii) administering to the human a therapeutically effective amount of an isolated tumor associated antigen (TAA).

4. A method of treating a human having a cancer, comprising:
i) administering to the human a therapeutically effective amount of a recombinant modified vaccinia Ankara (MVA) viral vector, comprising:
(a) a first nucleic acid sequence encoding a chimeric amino acid sequence, comprising (i) an extracellular fragment of mucin-1 (MUC-1), (ii) a transmembrane domain of a glycoprotein (GP) of Marburg virus, and (iii) an intracellular fragment of MUC-1; and
(b) a second nucleic acid encoding a Marburg virus VP40 matrix protein;
wherein both the first nucleic acid sequence and the second nucleic acid sequence are under control of promoters compatible with poxvirus expression systems, and wherein upon expression, the chimeric amino acid sequence and the VP40 matrix protein are capable of assembling together to form virus like particles (VLPs), and
ii) administering to the human a therapeutically effective amount of an oncolytic vaccinia virus.

5. The method of claim 4, wherein the human is further administered a therapeutically effective amount of an isolated MUC-1 tumor associated antigen.

6. The method of claim 1, wherein the TAA is selected from a group consisting of MUC-1, survivin, cyclin B1, and HPV or fragment thereof.

7. The method of claim 2, wherein the TAA is selected from a group consisting of MUC-1, survivin, cyclin B1, and HPV or fragment thereof.

8. The method of claim 4, wherein the extracellular MUC-1 fragment comprises the amino acid sequence selected from the group consisting of SEQ ID NO:1, SEQ ID NO:2, SEQ ID NO:3, SEQ ID NO:4, and SEQ ID NO:5.

9. The method of claim 1, wherein the TAA comprises a MUC-1 antigen derived from the amino acid sequence of SEQ ID NO:6 or SEQ ID NO:11.

10. The method of claim 3, wherein the TAA comprises a MUC-1 antigen derived from the amino acid sequence of SEQ ID NO:6 or SEQ ID NO:11.

11. The method of claim 5, wherein the isolated MUC-1 fragment comprises a MUC-1 Tripartite Immunotherapy (MTI) antigen comprising the amino acid sequence of SEQ ID NO:7.

12. The method of claim 1, wherein the TAA comprises a survivin antigen derived from the amino acid sequence of SEQ ID NO:8.

13. The method of claim 3, wherein the TAA comprises a survivin antigen derived from the amino acid sequence of SEQ ID NO:8.

14. The method of claim 1, wherein the TAA comprises a cyclin B1 antigen derived from the amino acid sequence of SEQ ID NO:9.

15. The method of claim 3, wherein the TAA comprises a cyclin B1 antigen derived from the amino acid sequence of SEQ ID NO:9.

16. The method of claim 1, further comprising administering to the human at least one chemotherapeutic agent, wherein the chemotherapeutic agent is administered 24 hours or less prior to, concurrent with, or 24 hours or less subsequently to the administration of the recombinant MVA.

17. The method of claim 3, further comprising administering to the human at least one chemotherapeutic agent, wherein the chemotherapeutic agent is administered 24 hours or less prior to, concurrent with, or 24 hours or less subsequently to the administration of the recombinant MVA.

18. The method of claim 4, further comprising administering to the human at least one chemotherapeutic agent, wherein the chemotherapeutic agent is administered 24 hours or less prior to, concurrent with, or 24 hours or less subsequently to the administration of the recombinant MVA.

19. The method of claim 1, further comprising administering to the human a anti-PD-1 or anti-PD-L1 monoclonal antibody.

20. The method of claim 3, further comprising administering to the human a anti-PD-1 or anti-PD-L1 monoclonal antibody.

21. The method of claim 5, further comprising administering to the human a anti-PD-1 or anti-PD-L1 monoclonal antibody.


SEQUENCE LISTING (23 sequences)

SEQ ID NO: 1 - protein, 10 aa, Homo sapiens
TSAPDTRPAP

SEQ ID NO: 2 - protein, 20 aa, Homo sapiens
AHGVTSAPDTRPAPGSTAPP

SEQ ID NO: 3 - protein, 20 aa, Homo sapiens
AHGVTSAPDNRPALGSTAPP

SEQ ID NO: 4 - protein, 40 aa, Homo sapiens
AHGVTSAPDTRPAPGSTAPPAHGVTSAPDNRPALGSTAPP

SEQ ID NO: 5 - protein, 100 aa, Homo sapiens
AHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPP

SEQ ID NO: 6 - protein, 475 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 7 - protein, 31 aa, Homo sapiens
SKKKKGCKLFAVWKITYKDTGTSAPDTRPAP

SEQ ID NO: 8 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 9 - protein, 433 aa, Homo sapiens
MALRVTRNSKINAENKAKINMAGAKRVPTAPAATSKPGLRPRTALGDIGNKVSEQLQAKMPMKKEAKPSATGKVIDKKLPKPLEKVPMLVPVPVSEPVPEPEPEPEPEPVKEEKLSPEPILVDTASPSPMETSGCAPAEEDLCQAFSDVILAVNDVDAEDGADPNLCSEYVKDIYAYLRQLEEEQAVRPKYLLGREVTGNMRAILIDWLVQVQMKFRLLQETMYMTVSIIDRFMQNNCVPKKMLQLVGVTAMFIASKYEEMYPPEIGDFAFVTDNTYTKHQIRQMEMKILRALNFGLGRPLPLHFLRRASKIGEVDVEQHTLAKYLMELTMLDYDMVHFPPSQIAAGAFCLALKILDNGEWTPTLQHYLSYTEESLLPVMQHLAKNVVMVNQGLTKHMTVKNKYATSKHAKISTLPQLNSALVQDLAKAVAKV

SEQ ID NO: 10 - DNA, 1608 bp, Homo sapiens
atgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtag

SEQ ID NO: 11 - protein, 535 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 12 - protein, 30 aa, Marburg virus
WWTSDWGVLTNLGILLLLSIAVLIALSCIC

SEQ ID NO: 13 - DNA, 1608 bp, Homo sapiens
atgacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctgtgagtatgaccagcagcgtactctccagccacagccccggttcaggctcctccaccactcagggacaggatgtcactctggccccggccacggaaccagcttcaggttcagctgccacctggggacaggatgtcacctcggtcccagtcaccaggccagccctgggctccaccaccccgccagcccacgatgtcacctcagccccggacaacaagccagccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccacggtgtcacctcggccccggacaccaggccggccccgggctccaccgcccccccagcccatggtgtcacctcggccccggacaacaggcccgccttgggctccaccgcccctccagtccacaatgtcacctcggcctcaggctctgcatcaggctcagcttctactctggtgcacaacggcacctctgccagggctaccacaaccccagccagcaagagcactccattctcaattcccagccaccactctgatactcctaccacccttgccagccatagcaccaagactgatgccagtagcactcaccatagcacggtacctcctctcacctcctccaatcacagcacttctccccagttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgtggtggacatccgactggggtgttcttactaacttgggcatcttactactattgtccatagctgtcttgattgctctatcctgtatttgtcgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgtag

SEQ ID NO: 14 - protein, 535 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVWWTSDWGVLTNLGILLLLSIAVLIALSCICRRKNYGQLDIFPARDTYHPMSEYPTYHTHGRYVPPSSTDRSPYEKVSAGNGGSSLSYTNPAVAATSANL

SEQ ID NO: 15 - DNA, 60 bp, Homo sapiens
gctcatggtgttacttcagcgcctgatacaagacctgcacctggatctacagctcctcct

SEQ ID NO: 16 - DNA, 60 bp, Homo sapiens
gcacatggtgtaacatctgctccagatacaagaccagctccaggttcaacagcacctcca

SEQ ID NO: 17 - DNA, 60 bp, Homo sapiens
gcgcatggtgttactagtgctccagatacaagacctgcgcctggaagtactgcaccacca

SEQ ID NO: 18 - DNA, 60 bp, Homo sapiens
gcacatggtgtaactagtgcgcctgatacaagaccagcgccaggatcaactgctcctcct

SEQ ID NO: 19 - DNA, 60 bp, Homo sapiens
gctcatggtgttacttcagcgcctgatacaagacccgcacccggatctaccgctccgcct

SEQ ID NO: 20 - DNA, 60 bp, Homo sapiens
gcacacggcgtcacatctgctcccgacactcgtccagctcctggtagcacagcacctcca

SEQ ID NO: 21 - DNA, 60 bp, Homo sapiens
gcgcatggagtaaccagtgcaccagatacccgacctgcgccgggcagtactgccccaccg

SEQ ID NO: 22 - DNA, 60 bp, Homo sapiens
gcccacggggtgacgagcgccccggacacgcgcccagctccagggtcaacggcgccccct

SEQ ID NO: 23 - DNA, 1633 bp, Homo sapiens
gcgctcccgggatgacacctggaacacaatctccattcttcctactactactattgacagtactaacagtagtaacaggatctggacatgcgtctagtacaccaggtggagagaaggaaacatctgcgactcaaagatcttctgtaccatcttctacagagaagaatgcggtatctatgacatctagtgtactatcttctcattctcctggatctggatcttctactacacaaggacaagatgtaacactagcgccagctacagaaccagcttctggatctgctgctacttggggtcaagatgttacttctgttccagtaacaagaccagcgctaggatctacaacaccaccagcgcatgatgtaacaagtgcgccagataataagccagcgcctggttctactgctccaccagctcatggtgttacttcagcgcctgatacaagacctgcacctggatctacagctcctcctgcacatggtgtaacatctgctccagatacaagaccagctccaggttcaacagcacctccagcgcatggtgttactagtgctccagatacaagacctgcgcctggaagtactgcaccaccagcacatggtgtaactagtgcgcctgatacaagaccagcgccaggatcaactgctcctcctgctcatggtgttacaagtgcacctgataatagacctgcgttgggatctactgcgcctccagttcataatgtaacatcagcgtctggaagtgcgtctggttctgcgtctacattggttcataatggtacatctgcgagagcgacaacaactccagcgtctaagtctacaccattctctattccatctcatcattctgatacaccaacaacattggcgagtcattctacaaagacagatgcgagttctacacatcattctactgtaccaccactaacatcttctaatcatagtacatctccacaactatctactggtgtatctttcttcttcctatcctttcatatttctaatctacagttcaattctagtttggaagatccatctacagattattatcaagaactacaaagagatatttctgaaatgtttctacaaatatataaacaaggaggatttctaggactatctaatattaagtttagaccaggatctgtagtagttcaactaactctagcgtttagagaaggtactattaatgtacatgatgttgaaacacagtttaatcaatataagacagaagcggcgtctagatataatctaacaatttctgatgtatctgtatctgatgttccatttccattctctgcgcaatctggtgctggtgtatggtggacatctgattggggagtactaactaatctaggaattctactattgctatctattgcggtactaattgcgctatcttgtatatgtagaagaaagaattatggacaactagatattttcccagcgagagatacttatcatccaatgtctgaatatccaacatatcatacacatggaagatatgtaccaccttcttcaacagatagatctccatatgagaaggtatctgcgggaaatggtggttcttctctatcttatacaaatccagcggtagcggcgacttctgcgaatctataataagtcgacgcgct